SEQUENCE LISTING (52 sequences)

SEQ ID NO: 1 - DNA, 736 bp, Murinae
gtgatccaggatccgaagaggcccggagcaggagcatggcgtcgtcggggtcggtgcagcagctgcccctggtgctgctgatgttgctgttggcgagtgcggcacgggccagactctacttccgctcgggccagacttgctaccatcccattcgcggggaccagctggctctgctggggcgcaggacttatcctcggccgcatgagtacctgtccccagcggatctccccaagaattgggactggagaaatgtgaacggtgtcaactatgccagcgtcaccaggaaccagcacatcccacagtactgtggttcctgctgggcccacggcagcaccagtgccatggcagaccgaatcaacatcaagaggaaaggtgcatggccctccatcctgctgtccgtacagaatgtcattgactgtggcaatgctggctcttgtgaagggggcaatgaccttccggtgtgggagtatgcccacaagcatggcatccccgatgagacctgcaacaactaccaggcaaggaccaagactgtgacaagtttaaccagtgtgggacctgcactgaattcaaagagtgtcacaccatccagaattacaccctctggagagtgggtgattacggtccctgtccgggagggagaagatgatggcgagatctatgccaatggtcccatcagctgcgggataatgggcaccagagatgatgtctaactacactgggggcatctatgctgagc

SEQ ID NO: 2 - DNA, 1404 bp, Murinae
aaaggaccgggcggggcgtcccgagcgcgtgggcctgcgggtcgggtcaagaggtcgaaggtgctgcgcgtgatccaggatccgaattggcccggagcaggagcatggcgtcgtcggggtcggtgcagcagctgcccctggtgctgctgatgttgctgttggcgagtgcggcacgggccagactctacttccgctcgggccagacttgctaccatcccattcgcggggaccagctggctctgctggggcgcaggacttatcctcggccgcatgagtacctgtccccagcggatctccccaagaattgggactggagaaatgtgaacggtgtcaactatgccagcgtcaccaggaaccagcacatcccacagtactgtggttcctgctgggcccacggcagcaccagtgccatggcagaccgaatcaacatcaagaggaaaggtgcatggccctccatcctgctgtccgtacagaatgtcattgactgtggcaatgctggctcttgtgaagggggcaatgaccttccggtgtgggagtatgcccacaagcatggcatccccgatgagacctgcaacaactaccaggccaaggaccaagactgtgacaagtttaaccagtgtgggacctgcactgaattcaaagagtgtcacaccatccagaattacaccctctggagagtgggtgattacggctccctgtccgggagggagaagatgatggccgagatctatgccaatggtcccatcagctgcgggataatggcaacagagatgatgtctaactacactgggggcatctatgctgagcaccaggaccaggccgttatcaaccacatcatctctgtagctggctggggtgtcagcaacgatggcatcgagtactggattgtccgaaattcatggggcgaaccctggggtgagaaaggctggatgaggatcgtgaccagcacctacaagggaggcacaggtgacagctacaaccttgccatcgagagtgcctgcacatttggggaccccattgtttaggtagatgtctctggaagcagcgctgtgaaccatgacagggaggggtgattaattactgacactggacatgtccagacagctataaacagtgcttgtggacatgaggaccagagtgtggactgcatcccgagaggagacggtaaaggatgaaacacaactgcactgggaccctccgccgtaccctccaggcctgcctcctccaccactgagccctccaggcctgcctcctcttctacagtgcttgccttcagccacccggagaagagagctatggtttaggacagctcaacttatcaccagatctggagccctggaatccatgggaggggggaacaagtccagactgcttaagaaatgagtaaaatatctggcttcccac

SEQ ID NO: 3 - protein, 306 aa, Murinae
MASSGSVQQLPLVLLMLLLASAARARLYFRSGQTCYHPIRGDQLALLGRRTYPRPHEYLSPADLPKNWDWRNVNGVNYASVTRNQHIPQYCGSCWAHGSTSAMADRINIKRKGAWPSILLSVQNVIDCGNAGSCEGGNDLPVWEYAHKHGIPDETCNNYQAKDQDCDKFNQCGTCTEFKECHTIQNYTLWRVGDYGSLSGREKMMAEIYANGPISCGIMATEMMSNYTGGIYAEHQDQAVINHIISVAGWGVSNDGIEYWIVRNSWGEPWGEKGWMRIVTSTYKGGTGDSYNLAIESACTFGDPIV

SEQ ID NO: 4 - DNA, 1480 bp, Homo sapiens
ctgggccgaggccgaggccggggcgggatccagagcgggagccggcgcgggatctgggactcggagcgggatccggagcgggacccaggagccggcgcggggccatggcgaggcgcgggccagggtggcggccgcttctgctgctcgtgctgctggcgggcgcggcgcagggcggcctctacttccgccggggacagacctgctaccggcctctgcggggggacgggctggctccgctggggcgcagcacatacccccggcctcatgagtacctgtccccagcggatctgcccaagagctgggactggcgcaatgtggatggtgtcaactatgccagcatcacccggaaccagcacatcccccaatactgcggctcctgctgggcccacgccagcaccagcgctatggcggatcggatcaacatcaagaggaagggagcgtggccctccaccctcctgtccgtgcagaacgtcatcgactgcggtaacgctggctcctgtgaagggggtaatgacctgtccgtgtgggactacgcccaccagcacggcatccctgacgagacctgcaacaactaccaggccaaggaccaggagtgtgacaagtttaaccaatgtgggacatgcaatgaattcaaagagtgccacgccatccggaactacaccctctggagggtgggagactacggctccctctctgggagggagaagatgatggcagaaatctatgcaaatggtcccatcagctgtggaataatggcaacagaaagactggctaactacaccggaggcatctatgccgaataccaggacaccacatatataaaccatgtcgtttctgtggctgggtggggcatcagtgatgggactgagtactggattgtccggaattcatggggtgaaccatggggcgagagaggctggctgaggatcgtgaccagcacctataaggatgggaagggcgccagatacaaccttgccatcgaggagcactgtacatttggggaccccatcgtttaaggccatgtcactagaagcgcagtttaagaaaaggcatggtgacccatgaccagaggggatcctatggttatgtgtgccaggctggctggcaggaactggggtggctatcaatattggatggcgaggacagcgtggcactggctgcgagtgttcctgagagttgaaagtgggatgacttatgacacttgcacagcatggctctgcctcacaatgatgcagtcagccacctggtgaagaagtgacctgcgacacaggaaacgatgggacctcagtcttcttcagcagaggacttgatattttgtatttggcaactgtgggcaataatatggcatttaagaggtgaaagagttcagacttatcaccattcttatgtcactttagaatcaagggtgggggagggagggagggagttggcagtttcaaatcgcccaagtgatgaataaagtatctggctctgcacgaga

SEQ ID NO: 5 - protein, 303 aa, Homo sapiens
MARRGPGWRPLLLLVLLAGAAQGGLYFRRGQTCYRPLRGDGLAPLGRSTYPRPHEYLSPADLPKSWDWRNVDGVNYASITRNQHIPQYCGSCWAHASTSAMADRINIKRKGAWPSTLLSVQNVIDCGNAGSCEGGNDLSVWDYAHQHGIPDETCNNYQAKDQECDKFNQCGTCNEFKECHAIRNYTLWRVGDYGSLSGREKMMAEIYANGPISCGIMATERLANYTGGIYAEYQDTTYINHVVSVAGWGISDGTEYWIVRNSWGEPWGERGWLRIVTSTYKDGKGARYNLAIEEHCTFGDPIV

SEQ ID NO: 6 - DNA, 646 bp, Murinae
tcctttcctagtctgtcttcagatgaaacctattctctgcttgtacaagaaccagtagccgtcctcaaggccaacagcgttggggagcgttacgaggtttagagacgtttagccagttagtttaccaagactctttcgggactttcaccatcaatgaatccagtatagctgattctccaagattccctcatagaggaattttaattgatacatctagacacttcctgcctgtgaagacaattttaaaaactctggatgccatggcttttaataagtttaatgttcttcactggcacatagtggacgaccagtctttcccttatcagagtaccacttttcctgagctaagcaataagggaagctactctttgtctcatgtctatacaccaaacgatgtccggatggtgctggagtacgcccggctccgagggattcgagtcataccagaatttgatacccctggccatacacagtcttggggcaaaggacagaaaaaccttctaactccatgttacaatcaaaaaactaaaactcaagtgtttgggcctgtagacccaactgtaaacacaacgtatgcattctttaacacatttttcaaagaaatcagcagtgtgtttccagatcagttcatccacttgggaggaga

SEQ ID NO: 7 - DNA, 1805 bp, Murinae
ggatgctttcttcccagcgacccagactggaaggttggtccaaagactgcctagccagactcgcggagcagtcatgccgcagtccccgcgtagcgcccccgggctgctgctgctgcaggcgctggtgtcgctagtgtcgctggccctagtggccccggcccgactgcaacctgcgctatggcccttcccgcgctcggtgcagatgttcccgcggctgttgtacatctccgcggaggacttcagcatcgaccacagtcccaattccacagcgggcccttcctgctcgctgctacaggaggcgtttcggcgatattacaactatgtttttggtttctacaagagacatcatggccctgctagatttcgagctgagccacagttgcagaagctcctggtctccattaccctcgagtcagagtgcgagtccttccctagtctgtcttcagatgaaacctattctctgcttgtacaagaaccagtagccgtcctcaaggccaacagcgtttggggagcgttacgaggtttagagacgtttagccagttagtttaccaagactctttcgggactttcaccatcaatgaatccagtatagctgattctccaagattccctcatagaggaattttaattgatacatctagacacttcctgcctgtgaagacaattttaaaaactctggatgccatggcttttaataagtttaatgttcttcactggcacatagtggacgaccagtctttcccttatcagagtaccacttttcctgagctaagcaataagggaagctactctttgtctcatgtctatacaccaaacgatgtccggatggtgctggagtacgcccggctccgagggattcgagtcataccagaatttgatacccctggccatacacagtcttggggcaaaggacagaaaaaccttctaactccatgttacaatcaaaaaactaaaactcaagtgtttgggcctgtagacccaactgtaaacacaacgtatgcattctttaacacatttttcaaagaaatcagcagtgtgtttccagatcagttcatccacttgggaggagatgaagtagaatttcaatgttgggcatcaaatccaaacatccaaggtttcatgaagagaaagggctttggcagcgattttagaagactagaatccttttatattaaaaagattttggaaattatttcatccttaaagaagaactccattgtttggcaagaagtttttgatgataaggtggagcttcagccgggcacagtagtcgaagtgtggaagagtgagcattattcatatgagctaaagcaagtcacaggctctggcttccctgccatcctttctgctccttggtacttagacctgatcagctatgggcaagactggaaaaactactacaaagttgagccccttaattttgaaggctctgagaagcagaaacaacttgttattggtggagaagcttgcctgtggggagaatttgtggatgcaactaaccttactccaagattatggcctcgagcaagcgctgttggtgagagactctggagccctaaaactgtcactgacctagaaaatgcctacaaacgactggccgtgcaccgctgcagaatggtcagccgtggaatagctgcacaacctctctatactggatactgtaactatgagaataaaatatagaagtgacagacgtctacagcattccagctatgatcatgttgattctgaaatcatgtaaattaagatttgttaggctgttttttttttaaataaaccatctttttattgattgaatctttct

SEQ ID NO: 8 - protein, 536 aa, Murinae
MPQSPRSAPGLLLLQALVSLVSLALVAPARLQPALWPFPRSVQMFPRLLYISAEDFSIDHSPNSTAGPSCSLLQEAFRRYYNYVFGFYKRHHGPARFRAEPQLQKLLVSITLESECESFPSLSSDETYSLLVQEPVAVLKANSVWGALRGLETFSQLVYQDSFGTFTINESSIADSPRFPHRGILIDTSRHFLPVKTILKTLDAMAFNKFNVLHWHIVDDQSFPYQSTTFPELSNKGSYSLSHVYTPNDVRMVLEYARLRGIRVIPEFDTPGHTQSWGKGQKNLLTPCYNQKTKTQVFGPVDPTVNTTYAFFNTFFKEISSVFPDQFIHLGGDEVEFQCWASNPNIQGFMKRKGFGSDFRRLESFYIKKILEIISSLKKNSIVWQEVFDDKVELQPGTVVEVWKSEHYSYELKQVTGSGFPAILSAPWYLDLISYGQDWKNYYKVEPLNFEGSEKQKQLVIGGEACLWGEFVDATNLTPRLWPRASAVGERLWSPKTVTDLENAYKRLAVHRCRMVSRGIAAQPLYTGYCNYENKI

SEQ ID NO: 9 - DNA, 1746 bp, Homo sapiens
ctgatccgggccgggcgggaagtcgggtcccgaggctccggctcggcagaccgggcggaaagcagccgagcggccatggagctgtgcgggctggggctgccccggccgcccatgctgctggcgctgctgttggcgacactgctggcggcgatgttggcgctgctgactcaggtggcgctggtggtgcaggtggcggaggcggctcgggccccgagcgtctcggccaagccggggccggcgctgtggcccctgccgctcttggtgaagatgaccccgaacctgctgcatctcgccccggagaacttctacatcagccacagccccaattccacggcgggcccctcctgcaccctgctggaggaagcgtttcgacgatatcatggctatatttttggtttctacaagtggcatcatgaacctgctgaattccaggctaaaacccaggttcagcaacttcttgtctcaatcacccttcagtcagagtgtgatgctttccccaacatatcttcagatgagtcttatactttacttgtgaaagaaccagtggctgtccttaaggccaacagagtttggggagcattacgaggtttagagacctttagccagttagtttatcaagattcttatggaactttcaccatcaatgaatccaccattattgattctccaaggttttctcacagaggaattttgattgatacatccagacattatctgccagttaagattattcttaaaactctggatgccatggcttttaataagtttaatgttcttcactggcacatagttgatgaccagtctttcccatatcagagcatcacttttcctgagttaagcaataaaggaagctattctttgtctcatgtttatacaccaaatgatgtccgtatggtgattgaatatgccagattacgaggaattcgagtcctgccagaatttgatacccctgggcatacactatcttggggaaaaggtcagaaagacctcctgactccatgttacagtagacaaaacaagttggactcttttggacctataaaccctactctgaatacaacatacagcttccttactacatttttcaaagaaattagtgaggtgtttccagatcaattcattcatttgggaggagatgaagtggaatttaaatgttgggaatcaaatccaaaaattcaagatttcatgaggcaaaaaggctttggcacagattttaagaaactagaatctttctacattcaaaaggttttggatattattgcaaccataaacaagggatccattgtctggcaggaggtttttgatgataaagcaaagcttgcgccgggcacaatagttgaagtatggaaagacagcgcatatcctgaggaactcagtagagtcacagcatctggcttccctgtaatcctttctgctccttggtacttagatttgattagctatggacaagattggaggaaatactataaagtggaacctcttgattttggcggtactcagaaacagaaacaacttttcattggtggagaagcttgtctatggggagaatatgtggatgcaactaacctcactccaagattatggcctcgggcaagtgctgttggtgagagactctggagttccaaagatgtcagagatatggatgacgcctatgacagactgacaaggcaccgctgcaggatggtcgaacgtggaatagctgcacaacctctttatgctggatattgtaaccatgagaacatgtaa

SEQ ID NO: 10 - protein, 556 aa, Homo sapiens
MELCGLGLPRPPMLLALLLATLLAAMLALLTQVALVVQVAEAARAPSVSAKPGPALWPLPLLVKMTPNLLHLAPENFYISHSPNSTAGPSCTLLEEAFRRYHGYIFGFYKWHHEPAEFQAKTQVQQLLVSITLQSECDAFPNISSDESYTLLVKEPVAVLKANRVWGALRGLETFSQLVYQDSYGTFTINESTIIDSPRFSHRGILIDTSRHYLPVKIILKTLDAMAFNKFNVLHWHIVDDQSFPYQSITFPELSNKGSYSLSHVYTPNDVRMVIEYARLRGIRVLPEFDTPGHTLSWGKGQKDLLTPCYSRQNKLDSFGPINPTLNTTYSFLTTFFKEISEVFPDQFIHLGGDEVEFKCWESNPKIQDFMRQKGFGTDFKKLESFYIQKVLDIIATINKGSIVWQEVFDDKAKLAPGTIVEVWKDSAYPEELSRVTASGFPVILSAPWYLDLISYGQDWRKYYKVEPLDFGGTQKQKQLFIGGEACLWGEYVDATNLTPRLWPRASAVGERLWSSKDVRDMDDAYDRLTRHRCRMVERGIAAQPLYAGYCNHENM

SEQ ID NO: 11 - DNA, 676 bp, Murinae
  misc_feature 604: n is a, c, g, or t
ggagctggtgggccggagcggcggcgccgccatgtccgacagcgagaagctcaacctggactccatcatcgggcgcctgctggaagtgcagggctcacggcctgggaagaacgtgcagctgacagagaacgagatccgtggtctgtgcctcaaatcccgggagattttcctgagccagcccattcttctggagcttgaggcgcccctcaagatctgtggtgacatccatggccagtactatgaccttctacggctgtttgagtatggtggcttccctccagagagcaactacctcttcttgggggattatgtagatcggggcaagcagtctttggagaccatctgcctgttgctggcctataagatcagatacccggagaatttctttctacttcgtgggaaccatgagtgtgccagcatcaaccgcatttatggcttctatgatgaatgcaagagaagatacaacatcaaactgtggaagacgttcactgactgcttcaactgcctgcccattgcagccattgtggatgagaagatcttctgctgccacgggggcctgtctccagacttgcaatccatggagcagattaggcgtattatgcgngccacagacgtgcctgaccagggcctactgtgtgatctcctgtggtctgaccctgacaagaaatagcctcca

SEQ ID NO: 12 - DNA, 1369 bp, Murinae
ggaggcaggagagggcccggagctggtgggccggagcggcggcgccgccatgtccgacagcgagaagctcaacctggactccatcatcgggcgcctgctggaagtgcagggctcacggcctgggaagaacgtgcagctgacagagaacgagatccgtggtctgtgcctcaaatcccgggagattttcctgagccagcccattcttctggagcttgaggcgcccctcaagatctgtggtgacatccatggccagtactatgaccttctacggctgtttgagtatggtggcttccctccagagagcaactacctcttcttgggggattatgtagatcggggcaagcagtctttggagaccatctgcctgttgctggcctataagatcagatacccggagaatttctttctacttcgtgggaaccatgagtgtgccagcatcaaccgcatttatggcttctatgatgaatgcaagagaagatacaacatcaaactgtggaagacgttcactgactgcttcaactgcctgcccattgcagccattgtggatgagaagatcttctgctgccacgggggcctgtctccagacttgcaatccatggagcagattaggcgtattatgcggcccacagacgtgcctgaccagggcctactgtgtgatctcctgtggtctgaccctgacaaggatgttcaaggctggggcgagaatgaccgtggtgtctcctttacctttggggctgaggtggtagccaagttcctgcacaagcatgatttggacctcatctgcagagcacatcaggttgtagaagatggctatgagttctttgccaagagacagttggtgacactcttctcagctcccaactactgtggagagtttgacaatgctggtgccatgatgagtgtggatgagaccctcatgtgttccttccagatcctcaagcccgctgataagaataagggcaagtatgggcagttcagcggcctgaaccccggaggccggcccatcactccaccccgcaattctgccaaagccaagaaatagcctccatgtgctgcccttctgccccagatcgtttgtacagaaatcatgctgccatgggtcacactggcctctcaggcccacccgtcacggggaacacacagcgttaagtgtctttcctttattttttaaagaatcaatagcagcatctaatctcccagggctccctcccaccagcacctgtggtggctgcaagtggaatcctggggccaaggctgcagctcagggcaatggcagaccagattgtgggtctccagccttgcatggctggcagccagatcctggggcaacccatctggtctcttgaataaaggtcaaagctggattctc

SEQ ID NO: 13 - protein, 330 aa, Murinae
MSDSEKLNLDSIIGRLLEVQGSRPGKNVQLTENEIRGLCLKSREIFLSQPILLELEAPLKICGDIHGQYYDLLRLFEYGGFPPESNYLFLGDYVDRGKQSLETICLLLAYKIRYPENFFLLRGNHECASINRIYGFYDECKRRYNIKLWKTFTDCFNCLPIAAIVDEKIFCCHGGLSPDLQSMEQIRRIMRPTDVPDQGLLCDLLWSDPDKDVQGWGENDRGVSFTFGAEVVAKFLHKHDLDLICRAHQVVEDGYEFFAKRQLVTLFSAPNYCGEFDNAGAMMSVDETLMCSFQILKPADKNKGKYGQFSGLNPGGRPITPPRNSAKAKK

SEQ ID NO: 14 - DNA, 993 bp, Homo sapiens
atgtccgacagcgagaagctcaacctggactcgatcatcgggcgcctgctggaagtgcagggctcgcggcctggcaagaatgtacagctgacagagaacgagatccgcggtctgtgcctgaaatcccgggagatttttctgagccagcccattcttctggagctggaggcacccctcaagatctgcggtgacatacacggccagtactacgaccttctgcgactatttgagtatggcggtttccctcccgagagcaactacctctttctgggggactatgtggacaggggcaagcagtccttggagaccatctgcctgctgctggcctataagatcaagtaccccgagaacttcttcctgctccgtgggaaccacgagtgtgccagcatcaaccgcatctatggtttctacgatgagtgcaagagacgctacaacatcaaactgtggaaaaccttcactgactgcttcaactgcctgcccatcgcggccatagtggacgaaaagatcttctgctgccacggaggcctgtccccggacctgcagtctatggagcagattcggcggatcatgcggcccacagatgtgcctgaccagggcctgctgtgtgacctgctgtggtctgaccctgacaaggacgtgcagggctggggcgagaacgaccgtggcgtctcttttacctttggagccgaggtggtggccaagttcctccacaagcacgacttggacctcatctgccgagcacaccaggtggtagaagacggctacgagttctttgccaagcggcagctggtgacacttttctcagctcccaactactgtggcgagtttgacaatgctggcgccatgatgagtgtggacgagaccctcatgtgctctttccagatcctcaagcccgccgacaagaacaaggggaagtacgggcagttcagtggcctgaaccctggaggccgacccatcaccccaccccgcaattccgccaaagccaagaaatag

SEQ ID NO: 15 - protein, 330 aa, Homo sapiens
MSDSEKLNLDSIIGRLLEVQGSRPGKNVQLTENEIRGLCLKSREIFLSQPILLELEAPLKICGDIHGQYYDLLRLFEYGGFPPESNYLFLGDYVDRGKQSLETICLLLAYKIKYPENFFLLRGNHECASINRIYGFYDECKRRYNIKLWKTFTDCFNCLPIAAIVDEKIFCCHGGLSPDLQSMEQIRRIMRPTDVPDQGLLCDLLWSDPDKDVQGWGENDRGVSFTFGAEVVAKFLHKHDLDLICRAHQVVEDGYEFFAKRQLVTLFSAPNYCGEFDNAGAMMSVDETLMCSFQILKPADKNKGKYGQFSGLNPGGRPITPPRNSAKAKK

SEQ ID NO: 16 - DNA, 702 bp, Murinae
ggcatgacaggcagtgagcaggtgatgagccaggttgtggatctctttagtgagggaataggctgctggagatatggctgtggtctacaaggaggctggggaactagcaaggagatgctctctcagctatcacagccttacagcaaagccactatctctttggattttgaaattttctctgccatgcctatgactattttaaaattgggcaaagtatatccatttcagaggggctttttctgtactgacaacagcgtgaagtacccgtaccatgacagtaccatcccgtcccgtatactcgccatactggggcttggcttacccattttctctatgagtatggagaatctctgtctgtttactttaatgtcttgcattcgaattcctttgtcggcaatccctacatagccaccatttacaaagccgtcggagccttttgttcggagtctcagctagtcagtccttgactgacatcgctaagtatactataggcagtttgcggccgcacttcttggctatctgtaacccagactggtcaaaaatcaactgcagtgatggctatattgaggactacatatgtcaagggaatgaagagaaagtcaaggagggcaggttgtctttctactcgggacactcttcattctctatgtactgcatgctgtttgtcgcactttatcttcaagccaggatgaagggagactg

SEQ ID NO: 17 - DNA, 1432 bp, Murinae
catccttagagctcgcccggcctgttggagagggcacagggcagcggagggcgattggccgcgacgagccagcactgagagagcaggcgcctgaggcgacagatcggcggccactcggtggcagggcggcccaatccaaactgccctggtccctgctcccgtcagtctaagaggctcgcagtcgcttggggcggccgccatcccgagggcggggctctgggaattgggtatctggaccgccgcggtctgttcctcccgccactcgcaccaggtggtgacaccatccagccggtgaccatgttcgacaagacgcggctgccgtacgtggccctcgatgtgatttgcgtgttgctggctggattgccttttgcaattcttacttcaaggcatacccccttccagcgaggaatattctgtaatgatgactccatcaagtacccttacaaggaagacaccataccttatgccttattaggtggaatagtcattccattctgtattatcgttatgagtattggagaatctctgtctgtttactttaatgtcttgcattcgaattcctttgtcggcaatccctacatagccaccatttacaaagccgtcggagcctttttgttcggagtctcagctagtcagtccttgactgacatcgctaagtatactataggcagtttgcggccgcacttcttggctatctgtaacccagactggtcaaaaatcaactgcagtgatggctatattgaggactacatatgtcaagggaatgaagagaaagtcaaggagggcaggttgtctttctactcgggacactcttcattctctatgtactgcatgctgtttgtcgcactttatcttcaagccaggatgaagggagactgggcaagactcttacgacccatgctccagtttgggctcattgctttttccatatatgtgggcctttctcgagtgtctgactacaaacaccactggagtgacgtcacagttggactcattcagggagctgctatggctatactggttgctttgtatgtatccgatttcttcaaggacacacattcttacaaagagagaaaggaagaggatccacacacgactctccatgaaaccgccagttcacggaactactgggcgctggcccgcttcaaaggcaacagctggaggctaaaggcagggggatgcgtattacttcctgctgtacagaccattctataaaggactgctgctatctatacctcctggatgcccattttatgtgtgtacagttacttctaacacaatgagtaacagttcaattaaagaaaatgaagcctgtcactaaaacactgtcccacctgtacatttttattgaaagacgctatgtacaaatgtgtatgttacatgccttctcagaatgatgttgacttaaatataataaaaagcttgtgaacca

SEQ ID NO: 18 - protein, 378 aa, Murinae
ESRRLRRQIGGHSVAGRPNPNCPGPCSRQSKRLAVAWGGRHPEGGALGIGYLDRRGLFLPPLAPGGDTIQPVTMFDKTRLPYVALDVICVLLAGLPFAILTSRHTPFQRGIFCNDDSIKYPYKEDTIPYALLGGIVIPFCIIVMSIGESLSVYFNVLHSNSFVGNPYIATIYKAVGAFLFGVSASQSLTDIAKYTIGSLRPHFLAICNPDWSKINCSDGYIEDYICQGNEEKVKEGRLSFYSGHSSFSMYCMLFVALYLQARMKGDWARLLRPMLQFGLIAFSIYVGLSRVSDYKHHWSDVTVGLIQGAAMAILVALYVSDFFKDTHSYKERKEEDPHTTLHETASSRNYWALARFKGNSWRLKAGGCVLLPAVQTIL

SEQ ID NO: 19 - DNA, 1626 bp, Homo sapiens
tcagcgggaggggctggaccccgcgttcctcctccctgccggtccccatccttaaagcgagagtctggacgccccgcctgtgggagagagcgccgggatccggacggggagcaaccggggcaggccgtgccggctgaggaggtcctgaggctacagagctgccgcggctggcacacgagcgcctcggcactaaccgagtgttcgcgggggctgtgaggggagggccccgggcgccattgctggcggtgggagcgccgcccggtctcagcccgccctcggctgctctcctcctccggctgggaggggccgtagctcggggccgtcgccagccccggcccgggctcgagaatcaagggcctcggccgccgtcccgcagctcagtccatcgcccttgccgggcagcccgggcagagaccatgtttgacaagacgcggctgccgtacgtggccctcgatgtgctctgcgtgttgctggcttccatgcctatggctgttctaaaattgggccaaatatatccatttcagagaggctttttctgtaaagacaacagcatcaactatccgtaccatgacagtaccgtcacatccactgtcctcatcctagtgggggttggcttgcccatttcctctattattcttggagaaaccctgtctgtttactgtaaccttttgcactcaaattcctttatcaggaataactacatagccactatttacaaagccattggaacctttttatttggtgcagctgctagtcagtccctgactgacattgccaagtattcaataggcagactgcggcctcacttcttggatgtttgtgatccagattggtcaaaaatcaactgcagcgatggttacattgaatactacatatgtcgagggaatgcagaaagagttaaggaaggcaggttgtccttctattcaggccactcttcgttttccatgtactgcatgctgtttgtggcactttatcttcaagccaggatgaagggagactgggcaagactcttacgccccacactgcaatttggtcttgttgccgtatccatttatgtgggcctttctcgagtttctgattataaacaccactggagcgatgtgttgactggactcattcagggagctctggttgcaatattagttgctgtatatgtatcggatttcttcaaagaaagaacttcttttaaagaaagaaaagaggaggactctcatacaactctgcatgaaacaccaacaactgggaatcactatccgagcaatcaccagccttgaaaggcagcagggtgcccaggtgaagctggcctgttttctaaaggaaaatgattgccacaaggcaagaggatgcatctttcttcctggtgtacaagcctttaaagacttctgctgctgctatgcctcttggatgcacactttgtgtgtacatagttacctttaactcagtggttatctaatagctctaaactcattaaaaaaactccaagccttccaccaaaacagtgccccacctgtatacatttttattaaaaaaatgtaatgcttatgtataaacatgtatgtaatatgctttctatgaatgatgtttgatttaaatataatacatattaaaatgtatgggagaaccaaa

SEQ ID NO: 20 - protein, 378 aa, Homo sapiens
GGPEATELPRLAHERLGTNRVFAGAVRGGPRAPLLAVGAPPGLSPPSAALLLRLGGAVARGRRQPRPGLENQGPRPPSRSSVHRPCRAARAETMFDKTRLPYVALDVLCVLLASMPMAVLKLGQIYPFQRGFFCKDNSINYPYHDSTVTSTVLILVGVGLPISSIILGETLSVYCNLLHSNSFIRNNYIATIYKAIGTFLFGAAASQSLTDIAKYSIGRLRPHFLDVCDPDWSKINCSDGYIEYYICRGNAERVKEGRLSFYSGHSSFSMYCMLFVALYLQARMKGDWARLLRPTLQFGLVAVSIYVGLSRVSDYKHHWSDVLTGLIQGALVAILVAVYVSDFFKERTSFKERKEEDSHTTLHETPTTGNHYPSNHQP

SEQ ID NO: 21 - DNA, 816 bp, Homo sapiens
atttattcccttttgctagctggattgccttttgcaattcttacttcaaggcatacccccttccaacgaggagtattctgtaatgatgagtccatcaagtacccttacaaagaagacaccataccttatgcgttattaggtggaataatcattccattcagtattatcgttattattcttggagaaaccctgtctgtttactgtaaccttttgcactcaaattcctttatcaggaataactacatagccactatttacaaagccattggaacctttttatttggtgcagctgctagtcagtccctgactgacattgccaagtattcaataggcagactgcggcctcacttcttggatgtttgtgatccagattggtcaaaaatcaactgcagcgatggttacattgaatactacatatgtcgagggaatgcagaaagagttaaggaaggcaggttgtccttctattcaggccactcttcgttttccatgtactgcatgctgtttgtggcactttatcttcaagccaggatgaagggagactgggcaagactcttacgccccacactgcaatttggtcttgttgccgtatccatttatgtgggcctttctcgagtttctgattataaacaccactggagcgatgtgttgactggactcattcagggagctctggttgcaatattagttgctgtatatgtatcggatttcttcaaagaaagaacttcttttaaagaaagaaaagaggaggactctcatacaactctgcatgaaacaccaacaactgggaatcactatccgagcaatcaccagccttga

SEQ ID NO: 22 - protein, 271 aa, Homo sapiens
IYSLLLAGLPFAILTSRHTPFQRGVFCNDESIKYPYKEDTIPYALLGGIIIPFSIIVIILGETLSVYCNLLHSNSFIRNNYIATIYKAIGTFLFGAAASQSLTDIAKYSIGRLRPHFLDVCDPDWSKINCSDGYIEYYICRGNAERVKEGRLSFYSGHSSFSMYCMLFVALYLQARMKGDWARLLRPTLQFGLVAVSIYVGLSRVSDYKHHWSDVLTGLIQGALVAILVAVYVSDFFKERTSFKERKEEDSHTTLHETPTTGNHYPSNHQP

SEQ ID NO: 23 - DNA, 840 bp, Murinae
  misc_feature 474: n is a, c, g, or t
ccgaagtaagtttgccagttttctgtcttatactgaggttcgccgggtcatggtgccagcctgactgagaagaggacgctcccgggaaacgaatgaggaaccacctcctcctgctgttcaagtacaggggcctggtgcgcaaagggaagaaaagcaaaagacgaaaatggctaaatttaagatccgtccagccactgcctctgactgcagtgacatcctgcgactgatcaaggaactggctaaatatgaatacatggaagatcaagtcattttaactgagaaagatctccaagaggatggctttggagaacaccccttctaccactgcctggttgcagaagtgcctaaagagcactggacccctgaaggacatagcattgttgggttcgccatgtactattttacctatgacccatggattggcaagttgctgtatcttgaagacttcttcgtgatgagtgattacagaggctntggtataggatcagaaattttgaagaatctaagccaggttgccatgaagtgtcgctgcagcagtatgcacttcttggtagcagaatggaatgaaccatctatcaacttctacaaaagaagaggtgcttcggatctgtccagtgaagagggatgggaggctcttcaagattgacaagagtacttgctaaaaatggcagcagaggagtgaggcgtgccggtgtagaacatgacaacctccattgtgctttagaataattctcagcttcccttgctttctatcttgtggtgtaggtgaaataatagagcgagccaccattccaaagctttattaccagtgacgtgttgcatgtttgaaatcggtctggt

SEQ ID NO: 24 - DNA, 1052 bp, Murinae
gctgcgcagtttccccgaagtaagtttgccagttttctgtcttatactgaggttcgccgggtcatggtgccagcctgactgagaagaggacgctcccgggaaacgaatgaggaaccacctcctcctgctgttcaagtacaggggcctggtgcgcaaagggaagaaaagcaaaagacgaaaatggctaaatttaagatccgtccagccactgcctctgactgcagtgacatcctgcgactgatcaaggaactggctaaatatgaatacatggaagatcaagtcattttaactgagaaagatctccaagaggatggctttggagaacaccccttctaccactgcctggttgcagaagtgcctaaagagcactggacccctgaaggacatagcattgttgggttcgccatgtactattttacctatgacccatggattggcaagttgctgtatcttgaagacttcttcgtgatgagtgattacagaggctttggtataggatcagaaattttgaagaatctaagccaggttgccatgaagtgtcgctgcagcagtatgcacttcttggtagcagaatggaatgaaccatctatcaacttctacaaaagaagaggtgcttcggatctgtccagtgaagagggatggaggctcttcaagattgacaaagagtacttgctaaaaatggcagcagaggagtgaggcgtgccggtgtagacaatgacaacctccattgtgctttagaataattctcagcttcccttgctttctatcttgtgtgtagtgaaataatagagcgagcacccattccaaagctttattaccagtgacgttgttgcatgtttgaaattcggtctgtttaaagtggcagtcatgtatgtggtttggaggcagaattcttgaacatcttttgatgaagaacaaggtggtatgatcttactatataagaaaaacaaaacttcattcttgtgagtcatttaaatgtgtacaatgtacacactggtacttagagtttctgttttgattcttttttttttaaataaactactctttgattt

SEQ ID NO: 25 - protein, 171 aa, Murinae
MAKFKIRPATASDCSDILRLIKELAKYEYMEDQVILTEKDLQEDGFGEHPFYHCLVAEVPKEHWTPEGHSIVGFAMYYFTYDPWIGKLLYLEDFFVMSDYRGFGIGSEILKNLSQVAMKCRCSSMHFLVAEWNEPSINFYKRRGASDLSSEEGWRLFKIDKEYLLKMAAEE

SEQ ID NO: 26 - DNA, 1111 bp, Homo sapiens
gcgcagctcttagtcgcgggccgactggtgtttatccgtcactcgccgaggttccttgggtcatggtgccagcctgactgagaagaggacgctcccgggagacgaatgaggaaccacctcctcctactgttcaagtacaggggcctggtccgcaaagggaagaaaagcaaaagacgaaaatggctaaattcgtgatccgcccagccactgccgccgactgcagtgacatactgcggctgatcaaggagctggctaaatatgaatacatggaagaacaagtaatcttaactgaaaaagatctgctagaagatggttttggagagcaccccttttaccactgcctggttgcagaagtgccgaaagagcactggactccggaaggtaacccctcgccctttccagaagccagagagaccaacattgttggttttgccatgtactattttacctatgacccgtggattggcaagttattgtatcttgaggacttcttcgtgatgagtgattatagaggtacgattgagctttggcataggatcagaaattctgaagaatctaagccaggttgcaatgaggtgtcgctggcagcatgcacttcttgggcagaatggaatgaaccatccatcaacttctataaaagaagaggtgcttctgatctgtccagtgaagagggttggagactgttcaagatcgacaaggagtacttgctaaaaatggcaacagaggagtgaggagtgctgctgtagatgacaacctccattctattttagaataaattcccaacttctcttgctttctatgctgtttgtagtgaaataatagaatgagcacccattccaaagctttattaccagtggcgttgttgcatgtttgaaatgaggtctgtttaaagtggcaatctcagatgcagtttggagagtcagatctttctccttgaatatctttcgataaacaacaaggtggtgtgatcttaatatatttgaaaaaaacttcattctcgtgagtcatttaaatgtgtacaatgtacacactggtacttagagtttctgtttgattcttttttaataaactactctttgatttaattctaaaaaaaaaaaaaaaaaagaca

SEQ ID NO: 27 - protein, 190 aa, Homo sapiens
EPPPPTVQVQGPGPQREEKQKTKMAKFVIRPATAADCSDILRLIKELAKYEYMEEQVILTEKDLLEDGFGEHPFYHCLVAEVPKEHWTPEGNPSPFPEARETNIVGFAMYYFTYDPWIGKLLYLEDFFVMSDYRGTIELWHRIRNSEESKPGCNEVSLAACTSWAEWNEPSINFYKRRGASDLSSEEGWR

SEQ ID NO: 28 - DNA, 745 bp, Murinae
aatctatggagcagattcggcgaattatgagaccaactgatgtaccagatcaaggtcttctttgtgatctttggtggtctgaccccgatgaaagatgtcttaggctggggtgaaaatgacagaggagtgtccttcacatttggtgcagaagtggttgcaaaatttctccataagcatgattcggatcttatatgtagagcccatcaggtggttgaagatggctatgagtttttcgcaaagaggcagttagtcactctgttgttctgcgagcccaactactgtggcgagtttgacaatgcaggcgccatgatgagtgtggatgagaccctcatgtgttccttccagattttaaagcctgcagagaaaaagaagcccaacgccacgagacctgtcacaccaccacggggtatgatcacaaagcaagcaaagaaatagatgtcacttgacactgcctggttgggacttgtaacatagcgttcataaccttcctttttaaactgtgatgtgctggtcagcttgcccaggtagacctgtctgtcgggccctcctccatttgattactgctggcacttgctggttatagcagcaagccaagcacttcattctcaagagagcatttggttctgaacctctgttccctttgtggacagctctgatgatggtgttaagctgtacaccctggcaggttatcctgtctgaggagaaagtgtacaattgatctttttttagtttagtataagtcatg

SEQ ID NO: 29 - DNA, 2127 bp, Murinae
gctgctgcgggagggtcggcggcgggacggcgatggcggatatcgacaaactcaacatcgacagcatcatccaacggctgctggaagtgagagggtccaagccaggcaagaatgtccagctccaggagaacgagatccgaggactctgcctgaagtctcgggagatcttcctcagtcagcctatccttttagaacttgaagcaccactcaagatatgtggtgacatccacgggcagtactatgatttgctccgtctgtttgaatacggtggctttcctccagagagcaactatttgtttctcggggactatgtggacaggggcaagcagtccctggagacaatctgcctcttgctggcctacaaaatcaagtatccggagaacttctttcttctcagagggaaccacgagtgcgccagcatcaataggatctacggattttatgatgagtgtaaaagaagatacaacattaagctgtggaaaacgttcacagactgttttaactgcttgccgatagcagccatcgtggacgagaagatattctgctgtcatggaggtttatcaccagatcttcaatctatggagcagattcggcgaattatgagaccaactgatgtaccagatcaaggtcttctttgtgatcttttgtggtctgaccccgataaagatgtcttaggctggggtgaaaatgacagaggagtgtccttcacatttggtgcagaagtggttgcaaaatttctccataagcatgatttggatcttatatgtagagcccatcaggtggttgaagatggctatgagttttttgcaaagaggcagttagtcactctgttttctgcacccaactactgtggcgagtttgacaatgcaggcgccatgatgagtgtggatgagaccctcatgtgttccttccagattttaaagcctgcagagaaaaagaagcccaacgccacgagacctgtcacaccaccacggggtatgatcacaaagcaagcaaagaaatagatgtcacttgacactgcctggttgggacttgtaacatagcgttcataaccttcctttttaaactgtgatgtgctggtcagcttgcccaggtagacctgtctgtcgggccctcctccatttgattactgctggcacttgctggttatagcagcaagccaagcacttcattctcaagagagcattttgttttgaacctctgttccctttgtggacagctctgatgatggtgttaagctgtacaccctggcaggttatcctgtctgaggagaaagtgtacaattgatctttttttaatttagtataagtcatgaataatgtaaatgcctgttttctttaggatataaagagagccttagagtgcgtgagtctctacatgtaattgtcataaatgcattctgttgatacaaaccactgtgaacaattttttttccagtttgtttgaaagggactgctttccctcattgtcttgtcatgtacaaactagtgtctgcagctgtggcagcaggagtgacctgcctgccgccagccctgcccagactatctgaagcacactccttcccactgcacatttaataatgattaaagccattcttttcaatgtctgtgattccttcctaaagccaaagtttctgttggactgtatggcacgccctggggatgaggtggccagggcatcgaggctgcgtgcacaggccgcctccctccgtggggcctcagaagcaggttattttaactagcaatagtggtatagtgctgagtaagctattaatgatggaagttaatgacactttgtacagttcccatatagtctattcactgagtgatctttttacagttggatcaggcctgaacccgtccattcagaaagcttcaaattatagaaacaacactgtcctatacgagtgaccgataatgctttctttggctacattctttattctgcggtgacattgaggcttataaatcaaaaggaactaacttgccgtccaccggtttatacagaactcacagtatctatgacttttttaaactacgacctgttaaatgaatctgtttgcacagatgcccgtgtacaatgccatgtgctgagaatggtttcagacttattaaatgcaagcttgtt

SEQ ID NO: 30 - protein, 323 aa, Murinae
MADIDKLNIDSIIQRLLEVRGSKPGKNVQLQENEIRGLCLKSREIFLSQPILLELEAPLKICGDIHGQYYDLLRLFEYGGFPPESNYLFLGDYVDRGKQSLETICLLLAYKIKYPENFFLLRGNHECASINRIYGFYDECKRRYNIKLWKTFTDCFNCLPIAAIVDEKIFCCHGGLSPDLQSMEQIRRIMRPTDVPDQGLLCDLLWSDPDKDVLGWGENDRGVSFTFGAEVVAKFLHKHDLDLICRAHQVVEDGYEFFAKRQLVTLFSAPNYCGEFDNAGAMMSVDETLMCSFQILKPAEKKKPNATRPVTPPRGMITKQAKK

SEQ ID NO: 31 - DNA, 993 bp, Homo sapiens; identical to SEQ ID NO: 14

SEQ ID NO: 32 - protein, 330 aa, Homo sapiens; identical to SEQ ID NO: 15

SEQ ID NO: 33 - DNA, 747 bp, Murinae
  misc_feature 298: n is a, c, g, or t
cgacaagtcatgctgctcgctatgaataaggttgaaggagacaacattagcggcgtttgcttcgttggcctgtatgacctggacgcctctcgctacttcgtccttctgcctctgtgcctctgcgtatttgttgagctgggagagtagcccagtggtacagcgcccacctggaatacttgaggacctggggttgtctcccagcactgcaaaaggaaaattcactgttacagtcttccttgcacttaaaccagctttgtctattgtttttttggtttggctttgttacttttgttgctgnttatttttgttgttgttgtttgtttgtttgagacagggtttttttgctagccctgactgtcctgaaactccctctgtagaccaggctggcctcaaacttacagagatccgcctgcctcagcctcccaagtgctgggaataatggtgtggtcaccaccgcccagccttttgtctatttttaaacttgaaagaaacaacagcccagatttcaaaaataatataatgcacttatacctaaaaaaacaaccaggagtgcccagttaataacattttttaaatgtggggatgggaagggcattagaggagtcttccttctattgaagattcattaaagtatttaagatatgccctttcactctttatataaatccaagatttttctttgctgaagtatttaaaacttttgtacctttatatgtagatatgaatttgaaaatatgcttatgtgta

SEQ ID NO: 34 - DNA, 2021 bp, Murinae
gggcaagaaacttcaacagaagtccacacctccccagaagcatccgtcaaagagggacgagcagaccgagcaaacactcccagcgccaaagatcgggactgtggggaatctgcagggcccagttccaagctctctgggaaccggaacggcagggaaagccgagcgggcggcctgaaggagagaagcaatggatcagagggggctccaagtgaaggaagggtaagtccaaagagcagcgttcctgagactggcctgatagactgcagcacttcacaggccgccagttctccagaaccaaccagcctcaagggctccacatctctgcctgttcactcagcttccagagctaggaaagagcagggtgctggcagccattccgacgcttgaagaaaactgtctcgttcccccagaagcacatgtatgttacactggagatgaccaactgatttgtcttataaaggccactgttgagctgggagagtagcccagtggtacagcgcccacctggaatacttgaggacctggggttgtctcccagcactgcaaaaggaaaattcactgttacagtcttccttgcacttaaaccagctttgtctattgtttttttggtttggcttttatttttgttgctgttatttttgttgttgtttgtttgtttttttgtttgtttgtttgagacagggtttctttgctagccctgactgtcctgaaactccctctgtagaccaggctggcctcaaacttacagagatctgcctgcctcagcctcccgagtgctgggaataatggtgtggtcaccactgcccagccttttgtctgtttttaaacttgaaagaaacaacagcccagatttcaaaaataatataatgcatttatacctaaaaaaccaaccaggagtgcccagttaataacactttttaaatgtggggatgggaagggcattagaggagtcttccttctattgaagattcattaaagtattttaagatatgctctttcactctttatataaatccaagatttttctttgctgaagtatttaaaacttttgtacctttatatgtagatatgaatttgaaaatatgcttatgtgtatttgaacttttgaaaatcctagagaattgaatcaaatatttttatgatgtttttctactattttagctactttgcgactgtgatagctgttacactggatttttaaaaaacttgtacagcagcctctttacagtaaaaagagtgggtgtcacactgaaaggtctgtaagaagtggtcacagccacccctaccttccccaaaaggaggaacttggtggcaggtccctccctgattggactgtccctttctttctgcatgttataaatcagcaggtaagatggtaggtttttacaagttaggccgagctgtcgattccccttttaagtgttgaattaggattgaattatggccatttgtagttgctcgtgcctgtctttattttagtattttatttcccgagacaggaactcactgtgtggtgctccttggctgtctggtgttcagtctgtcccaggcaggtcacagagatctccccctctgcagcccactcatctctcccaagccaccacactcagcttttatctgttttaaaaatttaaacttaaaaaaatgtttttggaatagtacaaacacattgtgttgtaaatttctttgatgctatgcaaaattcctatctgcatctaagcctgcaaaagaaaatgtgcgaagggcagagtcagagttgggcaggaagagtgtagtgcagcagatgcagcgtgaagacactgaaggtgctaagacagcgtctcagtgctggtcctccttaaggattatctcgccagcgaggttttcttagatactttgatcccattggagctctgttaaagtttaaaatgaaaattatcatgtactgtatgggaaatgtaaatactaacttttccacatatgtaaacttcagacacaaatttttttgtgtgttcttttcatcaataaaattttctttgtat

SEQ ID NO: 35 - protein, 709 aa, Murinae
MERSPFLLACILLPLVRGHSLFTCEPITVPRCMKMTYNMTFFPNLMGHYDQGIAAVEMGHFLHLANLECSPNIEMFLCQAFIPTCTEQIHVVLPCRKLCEKIVSDCKKLMDTFGIRWPEELECNRLPHCDDTVPVTSHPHTELSGPQKKSDQVPRDIGFWCPKHLRTSGDQGYRFLGIEQCAPPCPNMYFKSDELDFAKSFIGIVSIFCLCATLFTFLTFLIDVRRFRYPERPIIYYSVCYSIVSLMYFVGFLLGNSTACNKADEKLELGDTVVLGSKNKACSVVFMFLYFFTMAGTVWWVILTITWFLAAGRKWSCEAIEQKAVWFHAVAWGAPGFLTVMLLAMNKVEGDNISGVCFVGLYDLDASRYFVLLPLCLCVFVGLSLLLAGIISLNHVRQVIQHDGRNQEKLKKFMIRIGVFSGLYLVPLVTLLGCYVYELVNRITWEMTWFSDHCHQYRIPCPYQANPKARPELALFMIKYLMTLIVGISAVFWVGSKKTCTEWAGFFKRNRKRDPISESRRVLQESCEFFLKHNSKVKHKKKHGAPGPHRLKVISKSMGTSTGATTNHGTSAMAIADHDYLGQETSTEVHTSPEASVKEGRADRANTPSAKDRDCGESAGPSSKLSGNRNGRESRAGGLKERSNGSEGAPSEGRVSPKSSVPETGLIDCSTSQAASSPEPTSLKGSTSLPVHSASRARKEQGAGSHSDA

SEQ ID NO: 36 - DNA, 2039 bp, Homo sapiens
aggagacaacattagtggagtttgctttgttggcctttatgacctggatgcttctcgctactttgtactcttgccactgtgcctttgtgtgtttgttgggctctctcttcttttagctggcattatttccttaaatcatgttcgacaagtcatacaacatgatggccggaaccaagaaaaactaaagaaatttatgattcgaattggagtcttcagcggcttgtatcttgtgccattagtgacacttctcggatgttacgtctatgagcaagtgaacaggattacctgggagataacttgggtctctgatcattgtcgtcagtaccatatcccatgtccttatcaggcaaaagcaaaagctcgaccagaattggctttatttatgataaaatacctgatgacattaattgttggcatctctgctgtcttctgggttggaagcaaaaagacatgcacagaatgggctgggttttttaaacgaaatcgcaagagagatccaatcagtgaaagtcgaagagtactacaggaatcatgtgagtttttcttaaagcacaattctaaagttaaacacaaaaagaagcactataaaccaagttcacacaagctgaaggtcatttccaaatccatgggaaccagcacaggagctacagcaaatcatggcacttctgcagtagcaattactagccatgattacctaggacaagaaactttgacagaaatccaaacctcaccagaaacatcaatgagagaggtgaaagcggacggagctagcacccccaggttaagagaacaggactgtggtgaacctgcctcgccagcagcatccatctccagactctctggggaacaggtcgacgggaagggccaggcaggcagtgtatctgaaagtgcgcggagtgaaggaaggattagtccaaagagtgatattactgacactggcctggcacagagcaacaatttgcaggtccccagttcttcagaaccaagcagcctcaaaggttccacatctctgcttgttcacccggtttcaggagtgagaaaagagcagggaggtggttgtcattcagatacttgaagaacattttctctcgttactcagaagcaaatttgtgttacactggaagtgacctatgcactgttttgtaagaatcactgttacattcttcttttgcacttaaagttgcattgcctactgttatactggaaaaaatagagttcaagaataatatgactcatttcacacaaaggttaatgacaacaatatacctgaaaacagaaaatgtgcaggttaataatatttttttaatagtgtgggaggacagagttagaggaatcttccttttctatttatgaagattctactcttggtaagagtattttaagatgtactatgctattttacttttttgatataaaatcaagatatttctttgctgaagtatttaaatcttatccttgtatctttttatacatatttgaaaataagcttatatgtatttgaacttttttgaaatcctattcaagtatttttatcatgctattgtgatattttagcactttggtagcttttacactgaatttctaagaaaattgtaaaatagtcttcttttatactgtaaaaaaagatataccaaaaagtcttataataggaatttaactttaaaaacccacttattgataccttaccatctaaaatgtgtgatttttatagtctcgttttaggaatttcacagatctaaattatgtaactgaaataaggtgcttactcaaagagtgtccactattgattgtattatgctgctcactgatccttctgcatatttaaaataaaatgtcctaaagggttagtagacaaaatgttagtcttttgtatattaggccaagtgcaattgacttcccttttttaatgtttcatgaccacccattgattgtattataaccacttacagttgcttatattttttgttttaacttttgttttttaacatttagaatattacattttgtattatacagtacctttctcagacattttgtag

SEQ ID NO: 37 - protein, 706 aa, Homo sapiens
MEMFTFLLTCIFLPLLRGHSLFTCEPITVPRCMKMAYNMTFFPNLMGHYDQSIAAVEMEHFLPLANLECSPNIETFLCKAFVPTCIEQIHVVPPCRKLCEKVYSDCKKLIDTFGIRWPEELECDRLQYCDETVPVTFDPHTEFLGPQKKTEQVQRDIGFWCPRHLKTSGGQGYKFLGIDQCAPPCPNMYFKSDELEFAKSFIGTVSIFCLCATLFTFLTFLIDVRRFRYPERPIIYYSVCYSIVSLMYFIGFLLGDSTACNKADEKLELGDTVVLGSQNKACTVLFMLLYFFTMAGTVWWVILTITWFLAAGRKWSCEAIEQKAVWFHAVAWGTPGFLTVMLLAMNKVEGDNISGVCFVGLYDLDASRYFVLLPLCLCVFVGLSLLLAGIISLNHVRQVIQHDGRNQEKLKKFMIRIGVFSGLYLVPLVTLLGCYVYEQVNRITWEITWVSDHCRQYHIPCPYQAKAKARPELALFMIKYLMTLIVGISAVFWVGSKKTCTEWAGFFKRNRKRDPISESRRVLQESCEFFLKHNSKVKHKKKHYKPSSHKLKVISKSMGTSTGATANHGTSAVAITSHDYLGQETLTEIQTSPETSMREVKADGASTPRLREQDCGEPASPAASISRLSGEQVDGKGQAGSVSESARSEGRISPKSDITDTGLAQSNNLQVPSSSEPSSLKGSTSLLVHPVSGVRKEQGGGCHSDT

SEQ ID NO: 38 - DNA, 773 bp, Murinae
ctgaggtgctagcaccagcctggttgtctctggcgggcctgaagcaagcatggatcaagaggctgtgggcaacgttgtgctcctggcccttgtcaccctcatcagcgtggtccagaatgcgttctttgcccacaaggtggagcatgaaagcaaggcgcataatgggagaagcttccagaggaccgggactcttgcctttgagcgggtctacactgccaaccagaactgcgtagatgcgtaccccactttccttgtggtactctggactgcaggactactttgcagccaagtccctgcagccttcgccggactgatgtacctgtttgtgaggcaaaaatactttgtcggctatctgggagagagaactcagagcacccctggctacatcttcggcaagcggatcatcctgttcctgttcctcatgtccttcgccgggatactcaaccattacctcatcttcttcttcggaagcgactttgagaactacatcagaacggtaagcacgacgatctccccgctgcttctcatcccctgattgctggagacagagaaggacgctcaccagatcaatagagacgcatcataacgcaacgccgcgaaggcttctgctcctcttcaagctgtagatgctgtcaatcttgctgccctcggggctctgtggcatccgttaactttgcttttccgggaagaaaaatgtcttgtgctaagctccacccctcgaatgcggcggtgggccaggatttatgtctacatccagcctatacttctcctgg

SEQ ID NO: 39 - DNA, 852 bp, Murinae
ggaaggctgaggtgctagcaccagcctggttgtctctggcgggcctgaagcaagcatggatcaagaggctgtgggcaacgttgtgctcctggcccttgtcaccctcatcagcgtggtccagaatgtgttttttgcccactatgtggagcatgaaagcaatgcgcataatgggagaagcttccagaggaccgggactcttgcctttgagcgggtctacactgccaaccagaactgcgtagatgcgtaccccactttccttgtggtactctggactgcaggactactttgcagccaagtccctgccgccttcgccggactgatgtacctgtttgtgaggcaaaaatactttgtcggctatctgggagagagaactcagagcacccctggctacatcttcggcaagcggatcatcctgttcctgttcctcatgtccttcgccgggatactcaaccattacctcatcttcttcttcggaagcgactttgagaactacatcagaacggtaagcacgacgatctccccgctgcttctcatcccctgattgctggagacagagaaggacgctcaccagatcaatagagacgcatcataacgcaacgccgcgaaggcttctgctcctcttcaagctgtagatgctgtcaatcttgctgccctcggggctctgtggcatccgttaactttgcttttccgggaagaaaaatgtcttgtgctagctccacccctcgaatgcggcggtggcccaggatttattgtctacatccagcctatacttctcctggcttatcctgctttctgaagatgtcttgtaatcagacacgtgttttcctaaaataaagggtatagacaaaattt

SEQ ID NO: 40 - protein, 161 aa, Murinae
MDQEAVGNVVLLALVTLISVVQNVFFAHYVEHESNAHNGRSFQRTGTLAFERVYTANQNCVDAYPTFLVVLWTAGLLCSQVPAAFAGLMYLFVRQKYFVGYLGERTQSTPGYIFGKRIILFLFLMSFAGILNHYLIFFFGSDFENYIRTVSTTISPLLLIP

SEQ ID NO: 41 - DNA, 873 bp, Homo sapiens
acttccccttcctgtacagggcaggttgtgcagctggaggcagagcagtcctctctggggagcctgaagcaaacatggatcaagaaactgtaggcaatgttgtcctgttggccatcgtcaccctcatcagcgtggtccagaatggattctttgcccataaagtggagcacgaaagcaggacccagaatgggaggagcttccagaggaccggaacacttgcctttgagcgggtctacactgccaaccagaactgtgtagatgcgtaccccactttcctcgctgtgctctggtctgcggggctactttgcagccaagttcctgctgcgtttgctggactgatgtacttgtttgtgaggcaaaagtactttgtcggttacctaggagagagaacgcagagcacccctggctacatatttgggaaacgcatcatactcttcctgttcctcatgtccgttgctggcatattcaactattacctcatcttctttttcggaagtgactttgaaaactacataaagacgatctccaccaccatctcccctctacttctcattccctaactctctgctgaatatggggttggtgttctcatctaatcaatacctacaagtcatcataattcagctcttgagagcattctgctcttctttagatggctgtaaatctattggccatctgggcttcacagcttgagttaaccttgcttttccgggaacaaaatgatgtcatgtcagctccgccccttgaacatgaccgtggccccaaatttgctattcccatgcattttgtttgtttcttcacttatcctgttctctgaagatgttttgtgaccaggtttgtgttttcttaaaataaaatgcagagacatgtttt

SEQ ID NO: 42 - protein, 161 aa, Homo sapiens
MDQETVGNVVLLAIVTLISVVQNGFFAHKVEHESRTQNGRSFQRTGTLAFERVYTANQNCVDAYPTFLAVLWSAGLLCSQVPAAFAGLMYLFVRQKYFVGYLGERTQSTPGYIFGKRIILFLFLMSVAGIFNYYLIFFFGSDFENYIKTISTTISPLLLIP

SEQ ID NO: 43 - DNA, 803 bp, Murinae
ttcagctttatgggttggcttccttgactgcattttctgtcagttaactaaactccagactcatggattttctcgaccagaaaatcagactattttcctgaataatctactagaaacttttacggaacacatttcatgtttcctttgaagagttaagagaagaaagtatttgtaagaacaggaaaagaaacaaatactttgcaaataaactggctgctgctgtgaccacatctgaatagcaaaggcgatcgatcaagcgctgcggacaaaaggcctcctgtaagctgcactgcctgacaatggtaagctccaatggctcccagtgcccttatgacgactcctttaagtacactctgtacgggtgcatgttcagcatggtcttcgtgcttgggctgatatccaactgtgttgcgatatacattttcatctgtgccctcaaagtgagaaatgaaactacaacgtacatgattaacctggcaatgtcagatttacttttcgtctttactttgccatttcggattttttactttgcaacacggaattggccatttggagatctactctgtaagatttcagtaatgctgttttacaccaatatgtatgggaagcattctgttcttaacctgtatcagtgtagatcgatttctggcaattgtctacccatttaagtcaaagactttaagaaacgaaacgaaaatgcaaagaatcgtttgcattgcctgtgtggttcacagtgatgggaggaagtgcgctgcagttttctttcagtcgacccactctcaggggaacaatactcagaagct

SEQ ID NO: 44 - DNA, 1849 bp, Murinae
agagacagcccatctcacaatacagctggcaacctccgaaaggcctctccattcagcaagcgcgaacatgcttaggaatttatctgggatcccttaaacgactgcctatcgccgtccggaatcaatgtagaaatacaaagtttgagaataaaaagaaggaagaagtacccgaggacgacgggcggacggacgcacggcgagtgtttgtgactgaagtaaagctggtttggaccctggcggctgaagcacaagtttccacgcggactggtctggtccgacttggaacagtttttccttacactttcagctttatgggttggcttccttgactgcattttctgtcagttaactaaactccagactcatggattttctcgaccagaaaatcagactattttcctgaataatctactagaaacttttacggaacacatttcatgtttcctttgaagagttaagagaagaaagtatttgtaagaacaggaaaagaaacaaatactttgcaaataaactggctgctgctgtgaccacatctgaatagcaaaggcgatcgatcaagcgctgcggacaaaaggcctcctgtaagctgcactgcctgacaatggtaagctccaatggctcccagtgcccttatgacgactcctttaagtacactctgtacgggtgcatgttcagcatggtcttcgtgcttgggctgatatccaactgtgttgcgatatacattttcatctgtgccctcaaagtgagaaatgaaactacaacgtacatgattaacctggcaatgtcagatttacttttcgtctttactttgccatttcggattttttactttgcaacacggaattggccatttggagatctactctgtaagatttcagtaatgctgttttacaccaatatgtatggaagcattctgttcttaacctgtatcagtgtagatcgatttctggcaattgtctacccatttaagtcaaagactttaagaacgaaacgaaatgcaaagatcgtttgcattgctgtgtggttcacagtgatgggaggaagtgcgcctgcagttttctttcagtcgacccactctcaggggaacaatacctcagaagcctgctttgagaactttccagcggccacatggaaaacttatctctccaggattgtgattttcattgaaatagtgggcttttttatccctctcattttgaacgtaacttgttctagtatggtgctaagaactttaaataaacctgttacattaagtagaagcaaaatgaacaaaactaaggttttaaaaatgatttttgtccacttggtcatcttctgtttctgttttgtgccctacaacatcaacctcattttgtactcgctcatgaggacacagacctttgttaactgctctgtggtggcggcagtgaggaccatgtacccgatcactctctgcatcgctgtttccaactgctgctttgaccctattgtttactacttcacctcagacacaattcagaactcaataaaaatgaaaaactggtcggttagaagaagtgactccaggttctctgaagttcagggcactgagaattttatccaacacaacctacagaccttaaaaaataagatatttgataatgaatctgcaatataagctgcctgactaagccactgggactgctccgtgttcaactgtgaaaactgtgttcttgggaactatctctccggctccaacagaaaatatttttaaaggaagtttgtgtctgatgtgttaaacattaaaatatattctattcttgtatgcacgccattttactttcttgaaccactttaacgtgttttttcctcattaaaaaaaaaaaactcc

SEQ ID NO: 45 - protein, 316 aa, Murinae
DDSFKYTLYGCMFSMVFVLGLISNCVAIYIFICALKVRNETTTYMINLAMSDLLFVFTLPFRIFYFATRNWPFGDLLCKISVMLFYTNMYGSILFLTCISVDRFLAIVYPFKSKTLRTKRNAKIVCIAVWFTVMGGSAPAVFFQSTHSQGNNTSEACFENFPAATWKTYLSRIVIFIEIVGFFIPLILNVTCSSMVLRTLNKPVTLSRSKMNKTKVLKMIFVHLVIFCFCFVPYNINLILYSLMRTQTFVNCSVVAAVRTMYPITLCIAVSNCCFDPIVYYFTSDTIQNSIKMKNWSVRRSDSRFSEVQGTENFIQ

SEQ ID NO: 46 - DNA, 1035 bp, Homo sapiens
atggtaagcgttaacagctcccactgcttctataatgactcctttaagtacactttgtatgggtgcatgttcagcatggtgtttgtgcttgggttaatatccaattgtgttgccatatacattttcatctgcgtcctcaaagtccgaaatgaaactacaacttacatgattaacttggcaatgtcagacttgctttttgtttttactttacccttcaggattttttacttcacaacacggaattggccatttggagatttactttgtaagatttctgtgatgctgttttataccaacatgtacggaagcattctgttcttaacctgtattagtgtagatcgatttctggcaattgtctacccatttaagtcaaagactctaagaaccaaaagaaatgcaaagattgtttgcactggcgtgtggttaactgtgatcggaggaagtgcacccgccgtttttgttcagtctacccactctcagggtaacaatgcctcagaagcctgctttgaaaattttccagaagccacatggaaaacatatctctcaaggattgtaattttcatcgaaatagtgggattttttattcctctaattttaaatgtaacttgttctagtatggtgctaaaaactttaaccaaacctgttacattaagtagaagcaaaataaacaaaactaaggttttaaaaatgatttttgtacatttgatcatattctgtttctgttttgttccttacaatatcaatcttattttatattctcttgtgagaacacaaacatttgttaattgctcagtagtggcagcagtaaggacaatgtacccaatcactctctgtattgctgtttccaactgttgttttgaccctatagtttactactttacatcggacacaattcagaattcaataaaaatgaaaaactggtctgtcaggagaagtgacttcagattctctgaagttcatggtgcagagaattttattcagcataacctacagaccttaaaaagtaagatatttgacaatgaatctgctgcctga

SEQ ID NO: 47 - protein, 344 aa, Homo sapiens
MVSVNSSHCFYNDSFKYTLYGCMFSMVFVLGLISNCVAIYIFICVLKVRNETTTYMINLAMSDLLFVFTLPFRIFYFTTRNWPFGDLLCKISVMLFYTNMYGSILFLTCISVDRFLAIVYPFKSKTLRTKRNAKIVCTGVWLTVIGGSAPAVFVQSTHSQGNNASEACFENFPEATWKTYLSRIVIFIEIVGFFIPLILNVTCSSMVLKTLTKPVTLSRSKINKTKVLKMIFVHLIIFCFCFVPYNINLILYSLVRTQTFVNCSVVAAVRTMYPITLCIAVSNCCFDPIVYYFTSDTIQNSIKMKNWSVRRSDFRFSEVHGAENFIQHNLQTLKSKIFDNESAA

SEQ ID NO: 48 - DNA, 814 bp, Murinae
gagcgcgcgtaagatggcactaccattttctgtcaaccttcggggtgcgtaatggcctctggccaggcctagcacatgtacctcacagaccaactggcaagcagccttcagggagctcgatccccaaacagccagtcaccacctctgtcccctcttcactgttggtcgtcagactgcctgagtggacagcaggctggtcgcgttgtattttcacttccttcctctgactggcttgctcttgtctctcagtctttcatcccaggcagctgcctgaggtaggtgaggaggatggtgagccaggcaggtctacaataaaggcagctctgtccggctccttctggctcgtgagtgtcaccggcctggaagactgagggaatggctcccctctctcctccccgtctttccccagttccttccctatgttggcccatgtgcccagggagttggaagcatcagggagaccctcttagtgtggggaaggaagtcagagaccattgacacagtgaagaggcaggatcatgtgttggaagcctgttagcaggaccaaggtgactcttgggagagactcttgtggacacaggccgtggtggcttgtcagaccttaaagggtccaggcccacccctgccaggatccctggtctgctttctccaggacacactgggacactgctgagtaatgagcagcttattacacacaatgggaagaggggcagagagggctgtgtcggttgagtctcggctgggactgaagtttgccataagtagtggttgtacatccaggagcctggctacctgtctttaccccttgaaggac

SEQ ID NO: 49 - DNA, 1164 bp, Murinae
ggtcgctatttcttggtgcgtgacatcaccgagaagatggacatactgggcaccttgaagagctgtggggctcccaacttccggcaggtgcggggaggcctccctgtgtttggcatgggacagcccagcctcttggggttcaggagggtcctgcagaaactccagacggacggactcaaggagtgcattatcttctgcgtgcgggaggagcctgtggtgttcttgcgcgctgaggaggactttgtgtcttacacacctcgagacaaggagagccttcatgagaacctcagggaccctagtccaggggtcaaggctgagaatctggagctggccatccagaaagagatccatgactttgcccaattgagagataatgtgtaccacgtataccacaacacagaggacctgcgcggggagccgcacaccgtggccatccgaggtgaggatggcgtgtgcgtgaccgaggaggtgtttaagcggccgctcttcctgcagcccacctacagataccaccgcctccccttgccagagcaaggggcccccctggaagcccagtttgatgcctttgtcagcgttcttcgggagacccccagccttctgccactcagagataaccacgggcctctgcctgccctcctgttcagctgccagtcaggtgtaggcagaaccaacctaggcatggtcctgggaaccctcgtcatgttccaccacagtaggaccacctcccagctagaggcagcctccccgttggccaaacccctgcccatggagcagtttcaggtgatccagggcttcatctgtaaggtgccacaggggaagaaaatggtggaggaggtggatcgagcgatcagtgcctgtgcagagttgcatgacctgaaggaggaggtcctaaaaaaccagaggaggctggaaagcttcaggccagagagccggggacaggaatgtggtagtcagcaagctgtccagcagagggcgctgtggagcctggagctgtacttctatctgctcctatttaactactatctgcatgagcagtaccccctggcctttgccctcagtttcagtcgatggctgtgtacccatcctgagctgtaccgtctgctggtggagctgaattcagtggggcccttggtccctggggacctcatcgccaagggctccctg

SEQ ID NO: 50 - protein, 388 aa, Murinae
GRYFLVRDITEKMDILGTLKSCGAPNFRQVRGGLPVFGMGQPSLLGFRRVLQKLQTDGLKECIIFCVREEPVVFLRAEEDFVSYTPRDKESLHENLRDPSPGVKAENLELAIQKEIHDFAQLRDNVYHVYHNTEDLRGEPHTVAIRGEDGVCVTEEVFKRPLFLQPTYRYHRLPLPEQGAPLEAQFDAFVSVLRETPSLLPLRDNHGPLPALLFSCQSGVGRTNLGMVLGTLVMFHHSRTTSQLEAASPLAKPLPMEQFQVIQGFICKVPQGKKMVEEVDRAISACAELHDLKEEVLKNQRRLESFRPESRGQECGSQQAVQQRALWSLELYFYLLLFNYYLHEQYPLAFALSFSRWLCTHPELYRLLVELNSVGPLVPGDLIAKGSL

SEQ ID NO: 51 - DNA, 4303 bp, Homo sapiens
ggctgctggcagactatgggtacaacggccagcacagcccagcagacggtctcggcaggcaccccatttgagggcctacagggcagtggcacgatggacagtcggcactccgtcagcatccactccttccagagcactagcttgcataacagcaaggccaagtccatcatccccaacaaggtggcccctgttgtgatcacgtacaactgcaaggaggagttccagatccatgatgagctgctcaaggctcattacacgttgggccggctctcggacaacacccctgagcactacctggtgcaaggccgctacttcctggtgcgggatgtcactgagaagatggatgtgctgggcaccgtgggaagctgtggggcccccaacttccggcaggtgcagggtgggctcactgtgttcggcatgggacagcccagcctctcagggttcaggcgggtcctccagaaactccagaaggacggacatagggagtgtgtcatcttctgtgtgcgggaggaacctgtgcttttcctgcgtgcagatgaggactttgtgtcctacacacctcgagacaagcagaaccttcatgagaacctccagggccttggacccggggtccgggtggagagcctggagctggccatccggaaagagatccacgactttgcccagctgagcgagaacacataccatgtgtaccataacaccgaggacctgtggggggagccccatgctgtggccatccatggtgaggacgacttgcatgtgacggaggaggtgtacaagcggcccctcttcctgcagcccacctacaggtaccaccgcctgcccctgcccgagcaagggagtcccctggaggcccagttggacgcctttgtcagtgttctccgggagacccccagcctgctgcagctccgtgatgcccacgggcctcccccagccctcgtcttcagctgccagatgggcgtgggcaggaccaacctgggcatggtcctgggcaccctcatcctgcttcaccgcagtgggaccacctcccagccagaggctgcccccacgcaggccaagcccctgcctatggagcagttccaggtgatccagagctttctccgcatggtgccccagggaaggaggatggtggaagaggtggacagagccatcactgcctgtgccgagttgcatgacctgaaagaagtggtcttggaaaaccagaagaagttagaaggtatccgaccggagagcccagcccagggaagcggcagccgacacagcgtctggcagagggcgctgtggagcctggagcgatacttctacctgatcctgtttaactactaccttcatgagcagtacccgctggcctttgccctcagtttcagccgctggctgtgtgcccaccctgagctgtaccgcctgcccgtgacgctgagctcagcaggccctgtggctccgagggacctcatcgccaggggctccctacgggaggacgatctggtctccccggacgcgctcagcactgtcagagagatggatgtggccaacttccggcgggtgccccgcatgcccatctacggcacggcccagcccagcgccaaggccctggggagcatcctggcctacctgacggacgccaagaggaggctgcggaaggttgtctgggtgagccttcgggaggaggccgtgttggagtgtgacgggcacacctacagcctgcggtggcctgggccccctgtggctcctgaccagctggagaccctggaggcccagctgaaggcccatctaagcgagcctcccccaggcaaggagggccccctgacctacaggttccagacctgccttaccatgcaggaggtcttcagccagcaccgcagggcctgtcctggcctcacctaccaccgcatccccatgccggacttctgtgccccccgagaggaggactttgaccagctgctggaggccctgcgggccgccctctccaaggacccaggcactggcttcgtgttcagctgcctcagcggccagggccgtaccacaactgcgatggtggtggctgtcctggccttctggcacatccaaggcttccccgaggtgggtgaggaggagctcgtgagtgtgcctgatgccaagttcactaagggtgaatttcaggtagtaatgaaggtggtgcagctgctacccgatgggcaccgtgtgaagaaggaggtggacgcagcgctggacactgtcagcgagaccatgacgcccatgcactaccacctgcgggagatcatcatctgcacctaccgccaggcgaaggcagcgaaagaggcgcaagaaatgcggaggctgcagctgcggagcctgcagtacttggagcgctatgtctgcctgattctcttcaacgcgtacctccacctggagaaggccgactcctggcagaggcccttcagcacctggatgcaggaggtggcatcgaaggctggcatctacgagatccttaacgagctgggcttccccgagctggagagcggggaggaccagcccttctccaggctgcgctaccggtggcaggagcagagctgcagcctcgagccctctgcccccgaggacttgctgtagggggccttactccctgtccccccacccacagggccccacgcaggcctggggtgtctgaggtgctcttggctgggagcggccctgaggggtgctggccttgaaatgattcccccacttcctggagagactgagcggagttgggagcctttttagaaagaactttttataggacagggagacagcacagccatcccttgcaaaccaccaaggtgtgtggctgacctccagggaggagcactcactggagtgctcacaaggtgcacactgctgtgtgtaccttgcagacaggccggcgttcagcctccaaggggctcactcccccagttgccaaacactgtggatctctctgtcctcttctcccctctctcagattggcctggcagcccctggcacagagcagacccggccactggtagctccccacttccttactcctgctgctctgccattgccgctccccttgttgctgcccaagcactgccctcgggcgtctggcagcctgaggtgggtggaggggacagtgttctggatagatctattatgtgaaaggcagcttcacccagttttctggactctcatgcccccatctccgacctgggagacttcaggaatgacaacctacccagcctggtggggctggcaggatggtggaggtttctcaaggagctggagacttcagggagcccctctcatggggaggaaagagcttccagggggcgaacgcagcacagaggaagaggcctgctccacttgtctgggaacctgggcaggaggcacagaggaagccaaggcctggagctgcaggtcccccggcatctctctctgtcccggcagcccaggatggcctggtgcccccacctgctgcagcaggagccccaaggagtgctagctgagggtggttgctggggtggtcctcatggacagtgaggtgtgcaagggtgcactgagggtggtgggaggggatcacctgggttccaggccatccttgctgagcatctttgagcctgccttccggtgggagcagaaaaggccagaccctgctgagttagaggctgctgggatccactgtttccacacagcgggaaggctgctgggaacaggtggcagagaagtgccatgtttgcgttgagccttgcagctcttccagctggggactggtgcttgctgaaacccaggagctgaacagtgaggaggctgtccaccttgcttggctcactgggaccaggaaagcctgtctttggttaggctcgtgtacttctgcaggaaaaaaaaaaaaggatgtgtcattggtcatgatatttgaaaaggggaggaggccgaagttgttcccatttatccagtattggaaaatatttgacccccttggctgaattcttttgcagaactactgtgtgtctgttcactaccttttcaggtttattgtttttatttttgcatgaattaagacgttttaatttctttgcagacaaggtctagatgcggagtcagagatgggactgaatggggagggatcctttgtgttctcatggttggctctgactttcagctgtgttgggaccactggctgatcacatcacctctctgcctcagtttccccatctgtaaaatgggagaataatacttgcctacctacctcacaggggtgttgtgaggattcatttgtgattttttttttttttgtacagagcttttaagcattaaaaacagctaaatgtga

SEQ ID NO: 52 - protein, 861 aa, Homo sapiens
GCWQTMGTTASTAQQTVSAGTPFEGLQGSGTMDSRHSVSIHSFQSTSLHNSKAKSIIPNKVAPVVITYNCKEEFQIHDELLKAHYTLGRLSDNTPEHYLVQGRYFLVRDVTEKMDVLGTVGSCGAPNFRQVQGGLTVFGMGQPSLSGFRRVLQKLQKDGHRECVIFCVREEPVLFLRADEDFVSYTPRDKQNLHENLQGLGPGVRVESLELAIRKEIHDFAQLSENTYHVYHNTEDLWGEPHAVAIHGEDDLHVTEEVYKRPLFLQPTYRYHRLPLPEQGSPLEAQLDAFVSVLRETPSLLQLRDAHGPPPALVFSCQMGVGRTNLGMVLGTLILLHRSGTTSQPEAAPTQAKPLPMEQFQVIQSFLRMVPQGRRMVEEVDRAITACAELHDLKEVVLENQKKLEGIRPESPAQGSGSRHSVWQRALWSLERYFYLILFNYYLHEQYPLAFALSFSRWLCAHPELYRLPVTLSSAGPVAPRDLIARGSLREDDLVSPDALSTVREMDVANFRRVPRMPIYGTAQPSAKALGSILAYLTDAKRRLRKVVWVSLREEAVLECDGHTYSLRWPGPPVAPDQLETLEAQLKAHLSEPPPGKEGPLTYRFQTCLTMQEVFSQHRRACPGLTYHRIPMPDFCAPREEDFDQLLEALRAALSKDPGTGFVFSCLSGQGRTTTAMVVAVLAFWHIQGFPEVGEEELVSVPDAKFTKGEFQVVMKVVQLLPDGHRVKKEVDAALDTVSETMTPMHYHLREIIICTYRQAKAAKEAQEMRRLQLRSLQYLERYVCLILFNAYLHLEKADSWQRPFSTWMQEVASKAGIYEILNELGFPELESGEDQPFSRLRYRWQEQSCSLEPSAPEDLL

(19) United States
(12) Patent Application Publication
(10) Pub. No.: US 2009/0036362 A1
(43) Pub. Date: Feb. 5, 2009

(54) Angiogenesis Affecting Polypeptides, Proteins, and Composition, and Methods of Use Thereof

(75) Inventors: Mats Hellstrom, Goteborg (SE); Elisabet Wallgard, Goteborg (SE); Mattias Kalen, Goteborg (SE)

Correspondence Address: WIGGIN AND DANA LLP;ATTENTION: PATENT DOCKETING, ONE CENTURY TOWER, P.O. BOX 1832, NEW HAVEN, CT, 06508-1832, US

(73) Assignee: AngioGenetics Sweden AB, Stockholm (SE)

(21) Appl. No.: 10/581,761

(22) PCT Filed: Dec. 6, 2004
(86) PCT No.: PCT/SE04/01814

§ 371 Date: Apr. 10, 2008

(30) Foreign Application Priority Data
Dec. 5, 2003 (SE) 0303268-7

(51) Int. Cl. A61K 38/00; C12N 15/11; C12N 15/00; C12N 5/06; C12Q 1/68; A61P 31/00; A61K 31/7088; C07K 16/18; A01K 67/027

(52) U.S. Cl. 514/12; 536/23.5; 536/24.5; 435/320.1; 435/325; 435/375; 514/44; 530/387.7; 800/13; 530/350; 530/391.3; 435/6

(57) ABSTRACT

The present invention relates to polynucleotides and proteins associated with vasculogenesis- and angiogenesis-related disorders. The invention further relates to methods for the identification of compounds that modulate the expression of angiogenesis-related genes and gene products and to using such compounds as therapeutic agents in the treatment of angiogenesis-related disorders. The invention also relates to methods for the diagnostic evaluation, genetic testing and prognosis of angiogenesis-related disorders, and to methods and compositions for the treatment these disorders.

### FIELD OF THE INVENTION

The invention relates to polypeptides and proteins encoded thereby which are involved in vasculogenesis and/or angiogenesis. These agents may be targeted when producing materials and methods used in the diagnosis and therapy of angiogenesis-related conditions. The invention further relates to such diagnostic and therapeutic methods and agents.

### BACKGROUND OF THE INVENTION

Both vasculogenesis, the development of an interactive vascular system comprising arteries and veins, and angiogenesis, the generation of new blood vessels, play a role in embryonic development. In contrast, angiogenesis is limited in a normal adult to the placenta, ovary, endometrium, and sites of wound healing. Angiogenesis, or its absence, plays an important role in the maintenance of a variety of pathological states. Some of these states are characterized by neovascularization, e.g., cancer, diabetic retinopathy, glaucoma, and age related macular degeneration. Others, e.g., stroke, infertility, heart disease, ulcers, and scleroderma, are diseases of angiogenic insufficiency.

Angiogenesis has a number of stages (see, e.g., Zhu and Witte, Invest New Drugs 17:195-212, 1999). The early stages of angiogenesis include endothelial cell protease production, migration of cells, and proliferation. The early stages also appear to require some growth factors, with VEGF, TGF-A and selected chemokines all putatively playing a role. Later stages of angiogenesis include population of the vessels with mural cells (pericytes or smooth muscle cells), basement membrane production, and the induction of vessel bed specializations. The final stages of vessel formation include what is known as remodelling wherein a forming vasculature becomes a stable, mature vessel bed. Thus, the process is highly dynamic, often requiring coordinated spatial and temporal waves of gene expression.

The complex angiogenesis process is subject to disruption through interference with one or more critical steps, and numerous disease states can result from or be exacerbated by the disruption. Unregulated angiogenesis can cause or worsen disease, for example, ocular neovascularization has been implicated as the most common cause of blindness and underlies the pathology of approximately 20 eye diseases. In certain previously existing conditions such as arthritis, newly formed capillary blood vessels invade the joints and destroy cartilage. In diabetes, new capillaries formed in the retina invade the vitreous humour, causing bleeding and blindness.

In addition to pathologies linked to unregulated angiogenesis, insufficient angiogenesis can also lead to undesirable results. Dead or damaged tissue can lead to numerous pathologies, revascularization of damaged tissues through a healthy, normal angiogenic process is essential to preventing further complications.

Therefore, new targets and treatments that inhibit or enhance angiogenesis are needed. Identification of more key factors involved in any stage of angiogenesis could lead to new diagnostic methods for pathologic conditions related to angiogenesis. Further, elucidation and understanding of the key factors involved in angiogenesis could form the basis for new methods to investigate potential therapies for angiogenesis-related conditions.

### BRIEF SUMMARY OF THE INVENTION

In accordance with the objects outlined above, the present invention discloses ten nucleic acid sequences and associated proteins which have key roles in vasculogenesis and/or angiogenesis. One object of the present invention is to present approaches for using the ten novel factors as molecular targets for therapeutic intervention in angiogenesis-related disease states. It is a further object of the present invention to provide materials and methods that can be used to screen compounds for the ability to modulate angiogenesis or angiogenesis-related conditions.

Therapeutics specifically targeting the sequences and proteins identified herein are also provided as agents or compositions which modulate vasculogenesis or angiogenesis.

According to one embodiment of the invention, an isolated nucleic acid molecule according to any one of SEQ ID NO:s 2, 4, 7, 9, 12, 14, 17, 19, 21, 24, 26, 29, 31, 34, 36, 39, 41, 44, 46, 49, and 51 or a fragment or analogue thereof is provided which has the ability to stimulate or inhibit at least one biological activity selected from the group consisting of vasculogenesis, angiogenesis, vascular permeability, endothelial cell proliferation, endothelial cell differentiation, endothelial cell migration, and endothelial cell survival, or an isolated nucleic acid molecule which hybridizes to one of the foregoing sequences under stringent conditions The invention is also directed to isolated nucleic acid molecules which hybridizes to a compliment of a nucleic acid molecule described above, and an isolated siRNA molecule of at least 19 base pairs targeted to an isolated nucleic acid molecule described above.

According to a further embodiment of the invention, an expression vector comprising one of the novel nucleic acids is provided. The nucleic acid may be operatively associated with a regulatory nucleic acid controlling the expression of the polypeptide encoded by the nucleic acid.

The invention further comprises host cells genetically engineered to contain a nucleic acid as described above, or transfected by an expression vector described above.

According to a further embodiment of the invention, a method of treating an angiogenesis-related condition in a cell, group of cells, or organism is provided, comprising administering an expression vector as described above to the cell, group of cells, or organism.

The invention further comprises antibodies with specific reactivity to the nucleic acid molecules described above. The antibodies may be polyclonal or monoclonal and may further comprise detectable labels, such as fluorescent labels.

According to a further embodiment of the invention, a transgenic, non-human animal is provided which has been genetically engineered to contain a transgene comprising a nucleic acid as described above, and animals which contain and express the transgene.

According to a further embodiment of the invention, a pharmaceutical composition is provided which comprises a nucleic acid sequence as described above. The compound may be administered to a cell, group of cells, or organism to affect vasculogenesis or angiogenesis. The effect may be to increase or decrease vasculogenesis or angiogenesis, and the method may be employed where the cells, group of cells, or organism has an angiogenesis-related disorder. Such angiogenesis-related disorders include cancer, retinopathy, macular degeneration, corneal ulceration, stroke, ischemic heart disease, infertility, ulcers, scleradoma, wound healing, ischemia, ischemic heart disease, myocardial infarction, myocardosis, angina pectoris, unstable angina, coronary arteriosclerosis, arteriosclerosis obliterans, Berger's disease, arterial embolism, arterial thrombosis, cerebrovascular occlusion, cerebral infarction, cerebral thrombosis, cerebral embolism, rubeosis proliferative vitreoretinopathy, chronic inflammation, inflammatory bowel disease, psoriasis, sarcoidosis, and rheumatoid arthritis.

According to a further embodiment of the present invention, an isolated polypeptide comprising a sequence of amino acids substantially corresponding to the amino acid sequence in any one of SEQ ID NO:s 3, 5, 8, 10, 13, 15, 18, 20, 22, 25, 27, 30, 32, 35, 37, 40, 42, 45, 47, 50, and 52 or a fragment or analogue thereof is provided which has the ability to affect angiogenesis in a cell, a group of cells, or an organism.

The invention further comprises host cells genetically engineered to express a polypeptide as described above, as well as antibodies specifically reactive with the polypeptides. The antibody may be polyclonal or monoclonal, and may further comprise a detectable label such as fluorescence.

According to a further embodiment of the invention, a transgenic, non-human animal is provided which has been genetically engineered to contain a transgene comprising a nucleic acid which encodes a polypeptide as described above, and animals that contain and express the transgene.

The invention further provides pharmaceutical compositions comprising an isolated polypeptide as described above. The pharmaceutical composition may be administered to a cell, group of cells, or organism in order to affect vasculogenesis or angiogenesis therein. Vasculogenesis or angiogenesis may be increased or decreased. The cell, group of cells, or organism may have an angiogenesis-related disorder. Representative angiogenesis-related disorders are noted above.

According to a further embodiment of the invention, a method of detecting an angiogenesis-related transcript in a cell in a patient is provided, the method comprising contacting a biological sample from the patient with a polynucleotide that selectively hybridizes to a sequence at least 80% identical to a sequence according to any one of SEQ ID NO:s 2, 4, 7, 9, 12, 14, 17, 19, 21, 24, 26, 29, 31, 34, 36, 39, 41, 44, 46, 49, and 51 wherein an angiogenesis-related transcript is detected where hybridization is detected. The polynucleotide may comprise a sequence according to any one of SEQ ID NO:s 2, 4, 7, 9, 12, 14, 17, 19, 21, 24, 26, 29, 31, 34, 36, 39, 41, 44, 46, 49, and 51. The biological sample may be a tissue sample, or sample of isolated nucleic acids such as mRNA. According to this method, the nucleic acids may be amplified prior to contacting the biological sample with the polynucleotide. Further, the polynucleotide is immobilized on a solid surface.

According to a further embodiment of the present invention, a method of affecting at least one bioactivity selected from angiogenesis and vasculogenesis in a vertebrate organism is provided, where method comprises the step of administering an effective angiogenesis or vasculogenesis affecting amount of a nucleotide or polypeptide described herein to the organism. The organism may be mammal, such as mice, rats, rabbits, guinea pigs, cats, dogs, pigs, cows, monkeys, and humans. Vasculogenesis or angiogenesis may be enhanced, increased, inhibited, or decreased. This method may be used on organisms that have an angiogenesis-related disorder, such as those disorders described above.

According to a further embodiment of the invention, a transgenic increased angiogenesis laboratory animal is provided which comprises one or more cells in which the expression of a sequence according to any one of SEQ ID NO:s 2, 4, 7, 9, 12, 14, 17, 19, 21, 24, 26, 29, 31, 34, 36, 39, 41, 44, 46, 49, and 51 is upregulated. Transgenic decreased angiogenesis laboratory animals are also provided, which comprise one or more cells in which the expression of a sequence according to any one of SEQ ID NO:s 2, 4, 7, 9, 12, 14, 17, 19, 21, 24, 26, 29, 31, 34, 36, 39, 41, 44, 46, 49, and 51 is down regulated or absent.

The terms “angiogenesis-related condition” or “angiogenesis-related disease (state)” as used herein mean a condition which is marked by either an excess or a deficit of vessel development or which is improved by an increase or decrease in vessel development. Disorders associated with increased angiogenesis include, but are not limited to, cancer (including solid tumors, leukemias, and tumor metastases), benign tumors (including hemangiomas, acoustic neuromas, neurofibromas, trachomas, and pyogenic granulomas), retinopathy, macular degeneration, and corneal ulceration. Pathological states linked to decreased angiogenesis or states which can improve with increased angiogenesis include, but are not limited to, ischemic heart disease, infertility, ulcers, scleradoma, (insufficient) wound healing, ischemia, myocardial infarction, myocardosis, angina pectoris, unstable angina, coronary arteriosclerosis, arteriosclerosis obliterans (ASO), Berger's disease, arterial embolism, arterial thrombosis, cerebrovascular occlusion, cerebral infarction, cerebral thrombosis, cerebral embolism, and stroke. Other angiogenesis related diseases include, but are not limited to, diseases associated with rubeosis (neovasculariation of the angle) and diseases caused by the abnormal proliferation of fibrovascular or fibrous tissue including all forms of proliferative vitreoretinopathy, whether or not associated with diabetes, diseases with symptoms of chronic inflammation, such as inflammatory bowel disease, psoriasis, sarcoidosis and rheumatoid arthritis.

A “host cell” is a naturally occurring cell or a transformed cell that contains an expression vector and supports the replication or expression of the expression vector. Host cells may be cultured cells, explants, cells in vivo, and the like. Host cells may be prokaryotic cells such as E. coli, or eukaryotic cells such as yeast, insect, amphibian, or mammalian cells such as CHO, HeLa, and the like (see, e.g., the American Type Culture Collection catalog or web site, www.atcc.org).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. “Amino acid analogs” refers to compounds that have the same basic chemical structure as a naturally occurring amino acid. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “conservative modifications” or “conservatively modified variants” as used herein applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

As used herein, “label” or “detectable moiety” refers to a composition detectable by spectroscopic, photochemical, biochemical, immunochemical, chemical, or other physical means. Examples of such labels include ³²P, fluorescent dyes, electron-dense reagents, enzymes, biotin, digoxigenin, or haptens and proteins which can be made detectable, e.g., by incorporating a radiolabel into the peptide or used to detect antibodies specifically reactive with the peptide.

As used herein, “vector” or “expression vector” refers to a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The phrase “stringent hybridization conditions” as used herein refers to conditions under which sequences will hybridize. Stringent conditions are sequence-dependent and will be different in different circumstances. Skilled workers have access to significant amounts of descriptive material detailing reaction conditions that are appropriate for a given sequence. One example is Innis et al. (1990) PCR Protocols, A Guide to Methods and Applications, Academic Press, Inc. N.Y.).

As used herein, the terms “inhibitors,” “activators,” and “modulators” of angiogenic polynucleotide and polypeptide sequences and angiogeneic activity refer to inhibitory, activating, or modulating molecules. “Inhibitors” are compounds that, e.g., bind to, partially or totally block activity, decrease, prevent, delay activation, inactivate, desensitize, or down regulate the activity or expression of angiogenesis proteins, e.g., antagonists. “Activators” are compounds that increase, open, activate, facilitate, enhance activation, sensitize, agonize, or up regulate angiogenesis protein activity. Inhibitors, activators, or modulators include genetically modified versions of angiogenesis proteins, e.g., versions with altered activity, as well as naturally occurring and synthetic ligands, antagonists, agonists, antibodies, small chemical molecules and the like. Assays for inhibitors and activators include, e.g., expressing the angiogenic protein in vitro, in cells, or cell membranes, applying putative modulator compounds, and then determining the functional effects on activity, as described above.

### BRIEF DESCRIPTION OF THE FIGURES

FIG. 1 shows a ratio-ratio plot with the log₂ expression ratio of genes in a cDNA library when compared to embryonic brain endothelial cell portion versus embryonic brain left over portion and adult brain endothelial cell portion versus adult brain left over portion;

FIG. 2 shows a ratio-intensity plot with average intensity versus log₂ expression ratio of genes in a cDNA library when compared to embryonic brain, heart, and skin endothelial cell versus left over embryonic portions and all adult endothelial cells and left over portions;

FIG. 3 schematically depicts microarray data for gene OJC8009J7;

FIG. 4 shows a wild type zebrafish embryo at 28 hpf;

FIG. 5 shows a OJC8009J7 morphant embryo at 28 hpf;

FIG. 6 shows a wild type zebrafish embryo at 56 hpf;

FIG. 7 shows a OJC8009J7 morphant embryo at 56 hpf;

FIG. 8 shows a wild type zebrafish at 48-56 hpf;

FIG. 9 shows a OJC8009J7 morphant embryo at 48-56 hpf;

FIG. 10 schematically depicts microarray data for gene HUP8001K17;

FIG. 11 shows a HUP8001K17 morphant embryo at 28 hpf;

FIG. 12 shows a HUP8001K17 morphant embryo at 56 hpf;

FIG. 13 shows a HUP8001K17 morphant embryo at 48-56 hpf;

FIG. 14 schematically depicts microarray data for gene HUP8001K21;

FIG. 15 shows a HUP8001K21 morphant embryo at 28 hpf;

FIG. 16 shows a HUP8001K21 morphant embryo at 56 hpf;

FIG. 17 shows a HUP8001K21 morphant embryo at 48-56 hpf;

FIG. 18 schematically depicts microarray data for gene HUP8003D24;

FIG. 19 shows a HUP8003D24 morphant embryo at 48-56 hpf;

FIG. 20 schematically depicts microarray data for gene HUP8004N1;

FIG. 21 shows a HUP8004N1 morphant embryo at 28 hpf;

FIG. 22 shows a HUP8004N1 morphant embryo at 56 hpf;

FIG. 23 shows a HUP8004N1 morphant embryo at 48-56 hpf;

FIG. 24 schematically depicts microarray data for gene HUP8010A10;

FIG. 25 shows a HUP8010A10 morphant embryo at 28 hpf;

FIG. 26 shows a HUP8010A10 morphant embryo at 56 hpf;

FIG. 27 shows a HUP8010A10 morphant embryo at 48-56 hpf;

FIG. 28 schematically depicts microarray data for gene NOC8003L17;

FIG. 29 shows a NOC8003L17 morphant embryo at 28 hpf;

FIG. 30 shows a NOC8003L17 morphant embryo at 56 hpf;

FIG. 31 shows a NOC8003L17 morphant embryo at 48-56 hpf;

FIG. 32 schematically depicts microarray data for gene NOC8009C9;

FIG. 33 shows a NOC8009C9 morphant embryo at 28 hpf;

FIG. 34 shows a NOC8009C9 morphant embryo at 56 hpf;

FIG. 35 shows a NOC8009C9 morphant embryo at 48-56 hpf;

FIG. 36 schematically depicts microarray data for gene NOC8009G23;

FIG. 37 shows a NOC8009G23 morphant embryo at 28 hpf;

FIG. 38 shows a NOC8009G23 morphant embryo at 56 hpf;

FIG. 39 shows a NOC8009G23 morphant embryo at 48-56 hpf;

FIG. 40 schematically depicts microarray data for gene OJC8003C9;

FIG. 41 shows a OJC8003C9 morphant embryo at 28 hpf;

FIG. 42 shows a OJC8003C9 morphant embryo at 56 hpf; and

FIG. 43 shows a OJC8003C9 morphant embryo at 48-56 hpf.

### DETAILED DESCRIPTION

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention. Materials, the synthesis of which are not specifically described, are either commercially available or can be prepared using methods well known to those of skill in the art. Except as otherwise noted, all amounts including quantities, percentages, portions, and proportions, are understood to be modified by the word “about”, and amounts are not intended to indicate significant digits. Except as otherwise noted, the articles “a”, “an”, and “the” mean “one or more”. All documents cited are, in relevant part, incorporated herein by reference; the citation of any document is not to be construed as an admission that it is prior art with respect to the present invention.

Identification of Candidate Genes

A cDNA Library was prepared by collecting the mRNA from purely isolated adult and embryonic mice vascular fragments. The collected mRNA was used to develop cDNA libraries with a broad coverage of genes expressed in the vasculature. Because of the variety in mouse age, the vascular genes represented those active at different times and in different situations in the vasculature.

After creation of the cDNA library, microarrays were created by printing DNA from the cDNA library onto a solid support as known in the art. The microarrays were used to reveal the gene candidates through gene expression profiling. Select tissues from adult and E 18.5 embryonic mice were collected. Tissue selection was based on the amount and purity of RNA available for extraction. After tissues were removed, they were separated into two portions using antibodies or lectins. The first portion, the endothelial cell fraction or EC, contained endothelial cells as well as pericytes and vascular smooth muscle cells which are tightly associated with the vascular fragments. The second portion, also referred to as the left over portion or LO, were those cells remaining after the EC was isolated.

From adult mice, brain and heart tissues were used, both the EC and LO of each. From embryonic mice, brain, heart, and skin, both EC and LO of each, were utilized. The RNA from each fraction was extracted. A common reference RNA (Universal Mouse Reference RNA; Stratagene, Inc.) was employed at this stage for reference purposes. The isolated RNA and the reference RNA were reverse transcribed then amplified twice through two rounds of antisense RNA amplification. The isolated RNA was labelled with the fluorophore cyanine-3 and the reference RNA with cyanine-5.

After labelling, the RNA was assayed through hybridization with the microarrays described above. The hybridized microarrays were scanned and image analysis used to process the experimental data. Normalizing the data through a signal intensity-based normalization algorithm allowed for statistical evaluation of differentially expressed genes. Genes exhibiting differential expression were selected for further analysis.

Selection of Genes with Differential Expression

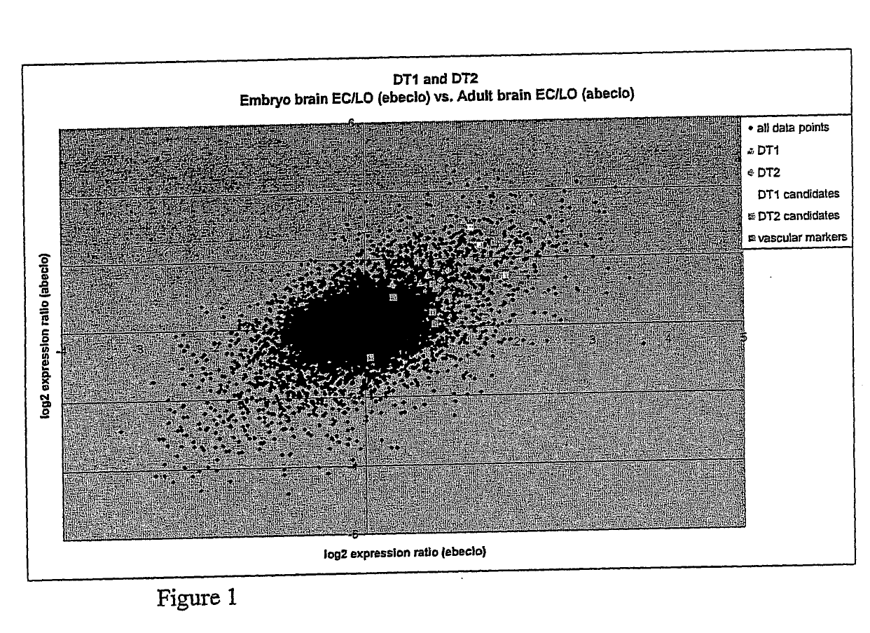

Using data collected as described above, certain genes were designated as selectively expressed in blood vessels. This was based on comparisons between adult and embryonic EC and LO values. FIG. 1 shows a ratio-ratio plot of the data values obtained through comparisons between embryonic brain EC and LO genes and between adult brain EC and LO genes. A comparison of total adult EC with total embryonic EC was also conducted, data not shown. Data points represented with ‘DT1 candidates’ or ‘DT2 candidates’ were generally upregulated (>0 log₂ expression ratio).

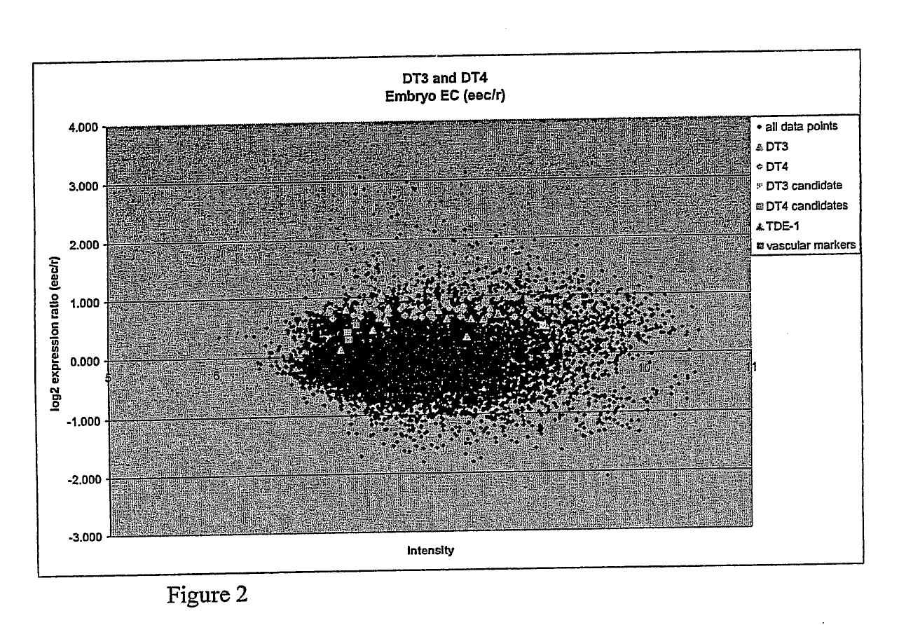

Other genes were designated as selectively expressed during angiogenesis through a different comparison of data. The total embryonic EC portion (i.e., brain, heart, and skin EC portions) was compared to all remaining tissues, including the total embryonic LO portion and all adult RNA (EC and LO of both brain and heart). FIG. 2 shows a ratio-intensity plot with the average intensity versus log₂ expression ratio of all genes. The data points marked DT3 candidates and DT4 candidates are those genes shown to be up regulated through this selective analysis. A comparison was also undertaken to analyze all EC portions versus all LO portions, data not shown.

A total of ten genes of interest were selected for further analysis. Specific expression data for each gene follows throughout and includes a graph showing that gene's expression profile.

Tables are used to show the intensity of the microarray signal, the log₂ expression ratio, p-value, and rank (rank given only for certain fields). The highest rank was awarded to the gene with the highest expression ratio value, the lowest rank was assigned to the gene with the lowest expression value, based on expression ratio values. P-values are given as a value from zero to one. Values close to one indicate a gene that is upregulated, whereas values close to zero indicate a statistically down regulated gene. A statistically significant p-value of 0.05 corresponds to a p-value of 0.05 or 0.95.

For graphs, tables, and text the abbreviation eec/r refers to embryonic EC portions versus all remaining portions, ec/lo refers to all EC portions versus all LO portions, abeclo refers to adult brain EC portion versus adult brain LO portion, abecebec refers to adult brain EC portion versus embryonic brain EC portion, aheclo refers to adult heart EC portion versus adult heart LO portion, ebeclo refers to embryonic brain EC portion versus embryonic brain LO portion, eheclo refers to embryonic heart EC portion versus embryonic heart LO portion, and eseclo refers to embryonic skin EC portion versus embryonic skin LO portion.

Evaluation of Selected Genes

Further analysis of selected genes was conducted through knockdown technology in zebrafish. The process involves the use of specific antisense oligonucleotides that block translation from targeted mRNA molecule(s). This allows for inhibition of the gene of interest and allows for a determination of gene function in the development and health of the zebrafish. Zebrafish share genes for vertebrate functions with mammalian vertebrates such as mice and humans. Studies have demonstrated that organ and/or tissue development in zebrafish can reliably predict effects in humans (See, inter alia, Shin and Fishman, From zebrafish to humans: Modular medical models, Ann. Rev. Genomics and Human Genet. 2002: 3: 311-340; Clark et al., An oligonucleotide fingerprint normalized and expressed sequence tag characterized zebrafish library, Genome Res 2001 September; 11(9):1594-602. Because of their rapid external development, zebrafish embryo development can be easily monitored and analyzed. The presence of a yolk sac helps provide data from the development of a critically deficient embryo further than that possible with other research organisms, such as mice.

To prepare the embryos, the zebrafish homolog of the target gene was identified. Then, a specific morpholino phosphorodiamidate oligonucleotide was designed to match the AUG initiation codon or splice acceptor/donor site of the target gene. To create a stock solution of morpholino, pellets containing 100 nmoles of the phosphorodiamidate oligonucleotides were dissolved in 33.3 μl milli-Q water, giving a concentration of 25 mg/ml, and stored at −20° C. To create injection solution, 8 μl of the stock solution was added to 92 μl of sterile-filtered 1×Danieu buffer (58 mM NaCl, 0.7 mM KCl, 0.4 mM MgSO₄, 0.6 mM Ca(NO₃)₂, 5 mM HEPES, pH 7.6) supplemented with 15 mM Tris-Cl, pH 8.0. The 2 mg/ml injection solution was also stored at −20° C.

During injection, the materials and embryos were maintained at approximately 28° C. Injection needles were calibrated so that injection times could optimally be within a range of 100-600 msec. Embryos from the one cell stage to the early eight cell stage were used. The morpholinos were microinjected into the yolk sac. Specific injection volumes, or effective dose of the morpholino, are described below. Typical initial doses included 3, 6, and 12 ng (1.5, 3, and 6 nl, respectively). Toxicity at the 3 nl dose resulted in subsequent doses of 0.5, 1, and 2 ng (1, 2, and 4 nl, respectively). Approximately 40 embryos were injected at each dose level, and approximately 40 embryos were retained as non-injected controls.

After the morpholinos were injected into fertilized egg cells, the embryos engineered to have a knockdown of the specific gene were allowed to develop (See Nasevicius and Ekker, Effective targeted gene ‘knockdown’ in zebrafish, Nature Genetics vol 26, October 2000.). The embryos were monitored throughout development, both by examining morphology and undertaking specific analysis and assays of developing tissues.

In addition to single morpholino injections, double morpholino injections were performed as well. Specific injection volumes for double injections are described below. At the end of the first post-injection day, with embryos at the blastula or gastrula stage, propyl thioracil (PTU) 2× solution was added to the embryos, doubling their suspension volume. 48 hours post fertilization (hpf) the double injected embryos were fixed with cadherin 5 (cdh5) for in situ hybridization.

When 20% or more of the double injected embryos displayed low effect defects in the vasculature observed with cdh5, or when 10% or more of the embryos displayed medium or high effect defects, then microangiopathy and in situ hybridization with fli-1, flk-1, flt-4, tie-1, tie-2, and cdh5 were conducted. At least 120 embryos were administered the double morpholino dose, of which at least 100 were harvested at 24 hpf for in situ hybridization with the above-noted molecular markers. Remaining embryos were used for microangiopathy.

Data specific to the evaluation of each of the ten targets are described below. In general, the morphology observations conducted at 24-28 hpf included an indication of whether the embryos exhibited general delay relative to control embryos. Further, cell death type and degree were recorded, general embryo shape and brain morphology were recorded as well. Finally, yolk sac edema, if present, was evaluated and recorded, as was heart morphology.

Also, at approximately 24 hpf, double morpholino embryos were evaluated for in situ hybridization of fli-1, flk-1, flt-4, tie-1, tie-2, and cdh5. Overall morphology and the degree of reduction of staining in the intersegmental vessels as compared to control embryos, correlating to a percentage of lost expression, were noted. Those embryos showing a loss of 1-35% of intersegmental expression were considered to have a low effect, those embryos showing a loss of 36-70% of intersegmental expression were considered to have a medium effect, and those embryos showing a loss of 71-100% of intersegmental expression were considered to have a high effect.

At 48-56 hpf various parameters were reviewed and recorded, such as general embryo shape, degree of cell death, blood circulation, and heart morphology. For the embryos fixed with cdh5, staining was evaluated throughout the vasculature as described immediately above.

Microangiopathy was also evaluated at 48 hpf in double morpholino embryos. In order to observe the blood vessels, the embryos were transferred into a tricaine solution and the sinus venosa/common cardinal vein was injected with 10 μl FITC-Dextran solution (2,000,000 Da, 20 mg/ml).

Gene OJC8009J7

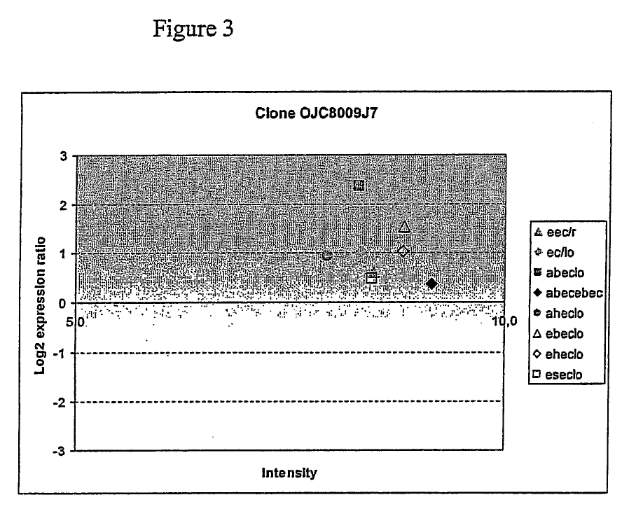

The gene having the sequence shown in SEQ ID NO:1 was identified as selectively expressed in blood vessels based on microarray data, see FIG. 3. Specific data are given below in Table 1. Using sequence and annotation databases the equivalent gene in mice (SEQ ID NO:2) and the human homolog (SEQ ID NO:4) was also deduced. Proteins encoded by these sequences are given at SEQ ID NO:s 3 and 5, respectively.

TABLE 1
Expression profile data for gene
OJC8009J7

| | intensity | log2 exp ratio | p-value | rank |
|---|---|---|---|---|
| eec/r | 8.5 | 0.62 | | 1445 |
| ec/lo | 8.3 | 1.03 | | 257 |
| abeclo | 8.3 | 2.37 | 1.00 | |
| abecebec | 9.2 | 0.38 | 0.84 | |
| aheclo | 7.9 | 0.93 | 1.00 | 5 |
| ebeclo | 8.8 | 1.53 | 1.00 | |
| eheclo | 8.8 | 1.03 | 1.00 | |
| eseclo | 8.5 | 0.48 | 0.99 | |

Based on this expression profile, the gene was further analyzed in zebrafish embryos. One corresponding zebrafish gene was identified for targeting. Two morpholinos were prepared, sz175 and sz176, each targeted to the zebrafish gene. Two (2)ng of sz175 morpholino and 12 ng of sz176 morpholino were administered to each fertilized egg. The embryos were allowed to develop. At 24 hpf a secondary in situ hybridization screen with six different probes was conducted.

Intersegmental expression was analyzed in the assay and results differed somewhat based on the probe used. The probe fli-1 revealed that 7% of the 14 morphant embryos assayed had low effects, and 29% had high effects, that is, loss of 71-100% of intersegmental expression. The probe flk-1, VEGF receptor 2, indicated that 25% of the 12 morphants reviewed had low effects and 8% had high effects. The probe tie-1 indicated that 11% of the 9 morphants observed had medium effects and 11% had high effects. The probe cdh5, VE cadherin, indicated that 19% of the 16 morphants observed had low effects, 6% had medium effects, and 19% had high effects. The probe flt-4, VEGF receptor 3, indicated that all 10 morphants observed were normal, and the probe tie-2 indicated that all 6 morphants observed were normal.

The following table, Table 2, summarizes this data.

TABLE 2
secondary in situ hybridization data

| Probe | Number analyzed | Results |
|---|---|---|
| fli-1 | 14 | 7% L, 29% M |
| flk-1 | 12 | 25% L, 8% H |
| tie-1 | 9 | 11% M, 11% H |
| cdh5 | 16 | 19% L, 6% M, 19% H |
| flt-4 | 10 | normal |
| tie-2 | 6 | normal |

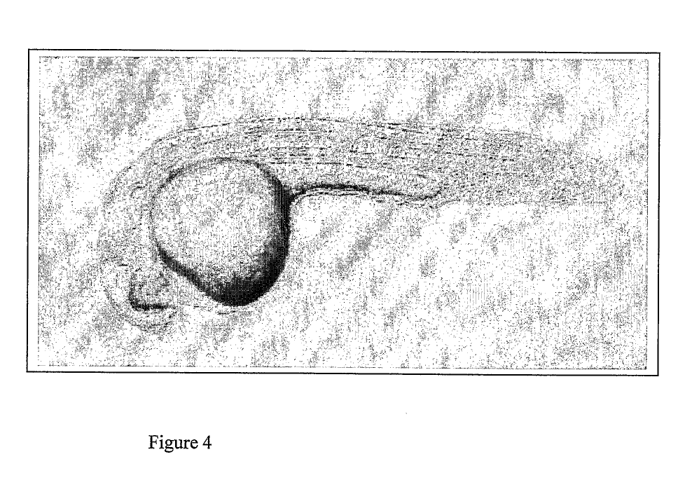

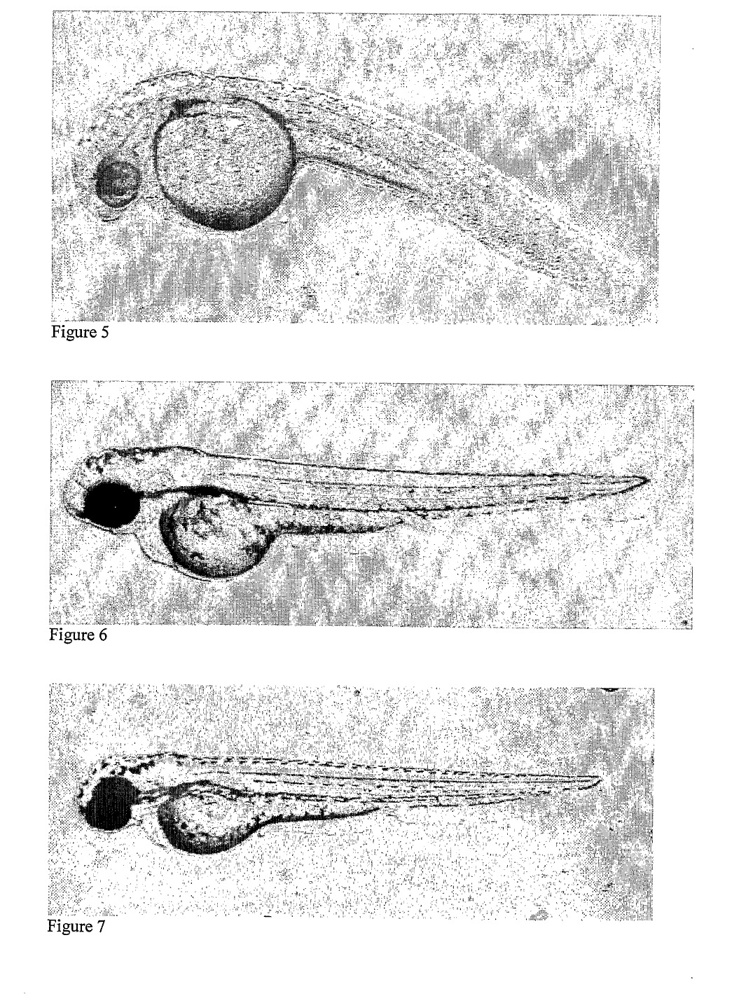

At 28 hpf embryos were observed morphologically. FIG. 5 shows a representative morphant embryo at 28 hpf. As evidenced from the figure, particularly when viewed in light of the 28 hpf wild type embryo of FIG. 4, the morphants exhibited normal morphology. At 56 hpf embryos again were observed for phenotypic characteristics, a representative morphant embryo is shown in FIG. 7. The normal morphology observed in the embryos can be easily understood when FIG. 7 is viewed in light of the 56 hpf wild type embryo of FIG. 6.

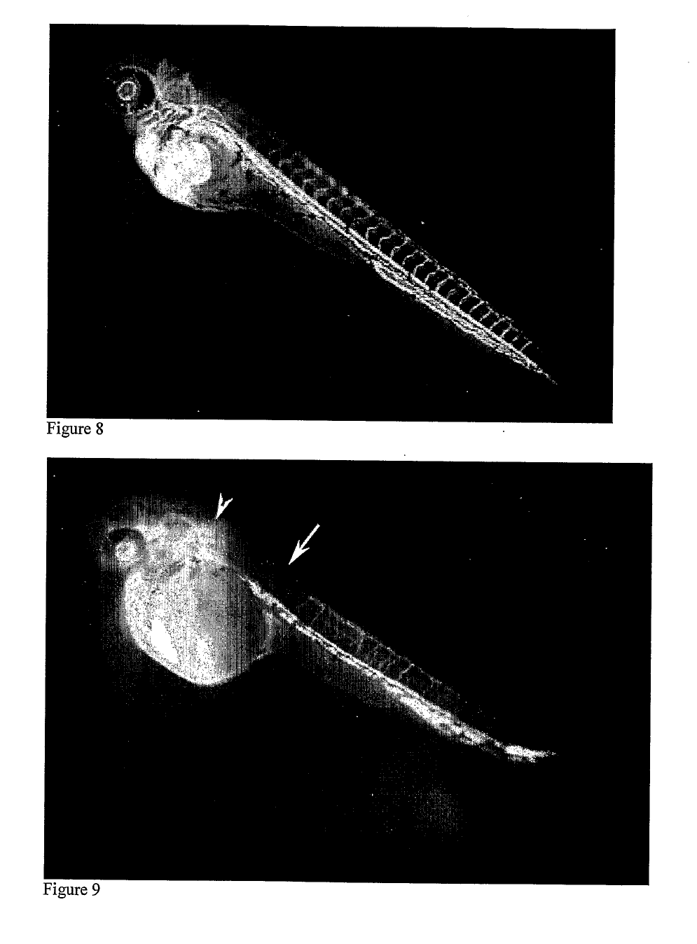

Additional analyses were conducted on 48-56 hpf morphant embryos. A primary in situ hybridization screen with cdh5 on 17 morphants showed 88% as normal. Medium effects of reduced intersegmental expression were seen in the other 12% of embryos. Microangiography on morphants was used to locate the presence of FITC-Dextran in various regions of the embryo, see FIG. 9, which can be compared with a wild type embryo at this time stage as shown in FIG. 8. Of the 33 embryos, none had FITC-Dextran in the heart and head combined, but 4% had it in the heart alone. Reduced intersegmental vasculature was seen in 15% of embryos. No leaky vasculature was observed. Normal embryos accounted for 81% of the sample. The leaks observed came from blood vessels in the posterior head as indicated by the arrowhead.

Gene HUP8001K17

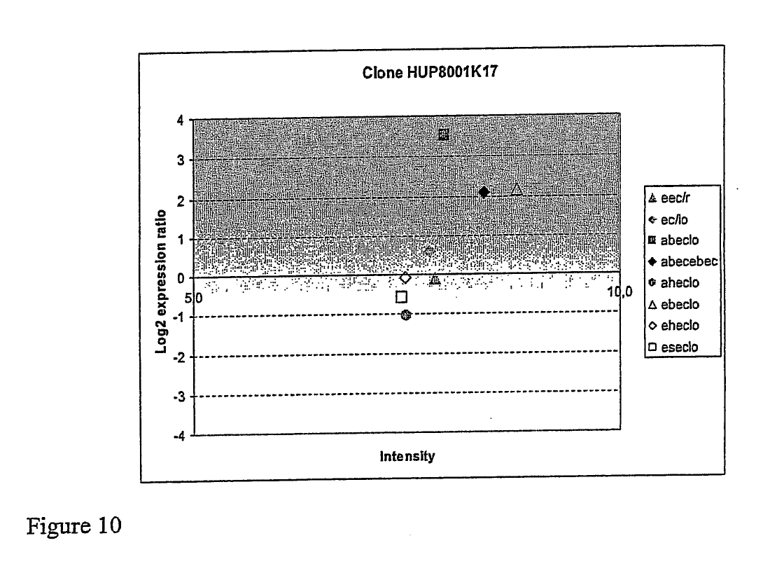

The gene having the sequence shown in SEQ ID NO:6 was identified as selectively expressed in blood vessels based on microarray data, see FIG. 10. Specific data are given below in Table 3. Using sequence and annotation databases the equivalent gene in mice (SEQ ID NO:7) and the human homolog (SEQ ID NO:9) was also deduced. Proteins encoded by these sequences are given at SEQ ID NO:s 8 and 10, respectively.

TABLE 3
Expression profile data for gene
HUP8001K17.

| | Intensity | log2 exp ratio | p-value | rank |
|---|---|---|---|---|
| eec/r | 7.8 | −0.11 | | 6732 |
| ec/lo | 7.8 | 0.59 | | 804 |
| abeclo | 7.9 | 3.52 | 1.00 | |
| abecebec | 8.4 | 2.10 | 1.00 | |
| aheclo | 7.5 | −1.05 | 0.00 | |
| ebeclo | 8.8 | 2.15 | 1.00 | |
| eheclo | 7.5 | −0.07 | 0.42 | 30 |
| Eseclo | 7.5 | −0.56 | 0.01 | |

Based on this expression profile, the gene was further analyzed in zebrafish embryos. One corresponding zebrafish gene was identified for targeting. Two different morpholinos were prepared, sz143 and sz144, each targeted to the zebrafish gene. Different amounts of morpholinos were administered as described below. The predetermined amount of each morpholino was administered to each fertilized egg. The embryos were allowed to develop. At 24 hpf secondary in situ hybridization screens with six different probes were conducted.

One screen was performed on embryos that received 1 ng of sz143 morpholino and 4 ng of sz144 morpholino. Four probes specifically selected to analyze axial and intersegmental vessel expression revealed the following: using the fli-1 probe, 6% of the 17 embryos analyzed had medium intersegmental expression effects. Another 18% had high effects. The probe flk-1, VEGF receptor 2, indicated that 20% of the 15 morphants reviewed had medium and 13% had high effects. When analyzed through the probe tie-1, 63% of the 16 morphants observed had high effects. The probe cdh5, VE cadherin, indicated that 4% of the 24 morphants observed had medium effects, and another 4% had high effects. The probe flt-4, VEGF receptor 3, indicated that all 14 morphants observed were normal, and the probe tie-2 showed all 18 observed morphants as normal.

The other screen was performed on embryos that received 1.5 ng of sz143 morpholino and 6 ng of sz144 morpholino. The probe fli-1 indicated that 42% of the 12 morphants analyzed had high effects. The flk-1 probe demonstrated that 23% of the 13 morphants observed had high effects. The tie-1 probe revealed 54% of 13 morphants had high effects. The probe cdh5 indicated that 15% of 27 morphants had medium effects and another 11% had high effects. The probe flt-4 indicated that all 14 morphants observed were normal. And the probe tie-2, showed all 18 observed morphants as normal.

The following Table 4 summarizes the foregoing data.

TABLE 4
secondary in situ hybridization data

| | Morphants with 1 ng sz143, | | Morphants with 1.5 ng | |
|---|---|---|---|---|
| | 4 ng sz144 | | sz143, 6 ng sz144 | |
| | Number | | Number | |
| Probe | analyzed | Results | analyzed | Results |
| fli-1 | 17 | 6% M, 18% H | 12 | 42% H |
| flk-1 | 15 | 20% M, 13% H | 13 | 23% H |
| tie-1 | 16 | 63% H | 13 | 54% H |
| cdh5 | 24 | 4% M, 4% H | 27 | 15% M, 11% H |
| flt-4 | 13 | normal | 14 | normal |
| tie-2 | 19 | normal | 18 | normal |

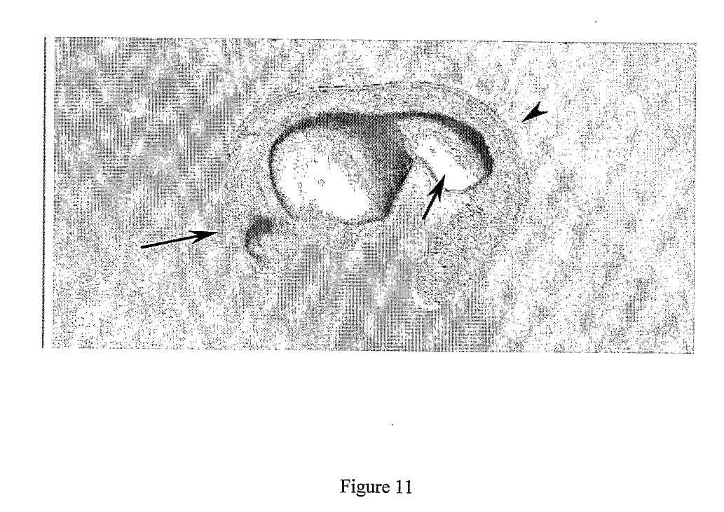

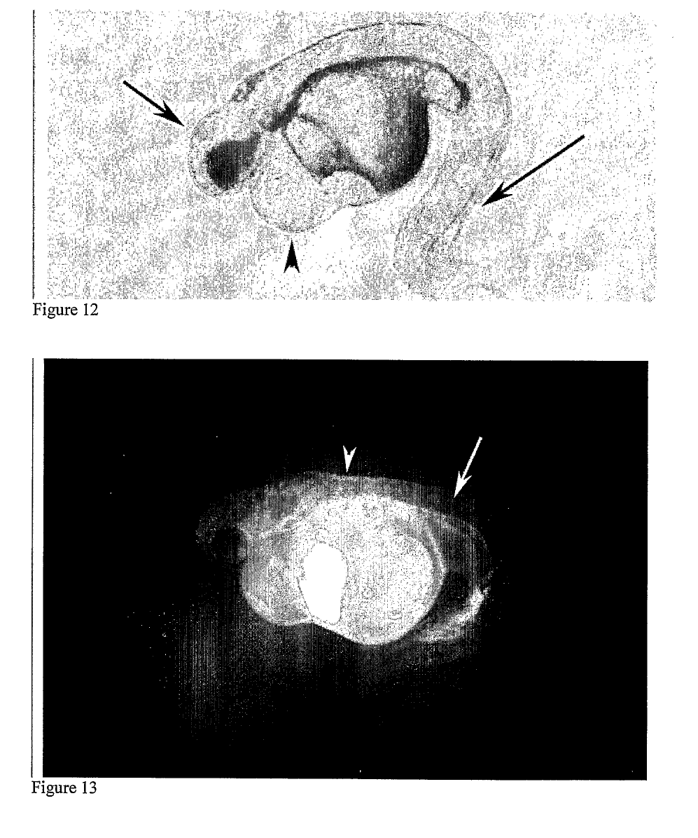

At 28 hpf embryos were observed morphologically. The wild type embryos, used as control, showed normal morphology as expected. As indicated previously, FIG. 4 shows a wild type embryo at 28 hpf. The morphant embryos received a 1.5 ng dose of sz143 and a 6 ng dose of sz144, all did not exhibit normal morphology. A representative embryo is shown in FIG. 11. Twenty embryos were observed, 50% of them showed a curly down body, indicated by the arrowhead in FIG. 11, with yolk tube extension, indicated with a short arrow. Mild cell death was observed in 60% of the embryos, as shown by the long arrow in FIG. 11. Finally, 50% of the embryos had yolk cell edema.

At 56 hpf embryos were again observed, for reference a wild type embryo is shown in FIG. 6. A morphant at the corresponding stage is shown in FIG. 12. Twenty (20) embryos were observed, 90% had a curly down body as shown by the long arrow in FIG. 12, with reduced head as indicated by the short arrow. Pericardial edema, shown by the arrowhead, was observed in 90% of the embryos and reduced blood flow was also seen in 90% of the embryos.

Additional analyses were conducted on 48-56 hpf morphant embryos. A primary in situ hybridization screen with cdh5 on 18 morphants which had received 1 ng sz143 and 3 ng sz144 revealed that 28% had reduced intersegmental expression, at a low effect level, with short and curly tails. The remaining 72% were normal. The same in situ hybridization screen was conducted using 11 morphants which had received 2 ng sz143 and 6 ng sz144. This revealed that 9% had reduced intersegmental expression, at a low effect level, with very short tails. The remaining 91% were normal.

Microangiography on 26 morphants which had received 1.5 ng of sz143 and 6 ng of sz144 was used to locate the presence of FITC-Dextran in various regions of the embryo. No FITC-Dextran was observed in the heart, but 31% of the embryos had FITC-Dextran in the head and heart. A total of 27% of the morphants had reduced intersegmental vasculature, and leaky vasculature was observed in 35% of the embryos. Only 42% of the embryos appeared normal. The combined percentages are greater than 100% since some embryos exhibited more than one non-normal feature. FIG. 13 shows a representative of the 48-56 hpf embryos analyzed. The arrow points to an area of reduced intersegmental vasculature, and the arrowhead indicates a point of leaky vasculature. For reference, a wild type embryo at this time stage is shown in FIG. 8. The experimental data reveal that the gene is expressed by scattered cells in many organs, but most clearly seen in the CNS.

Gene HUP8001K21

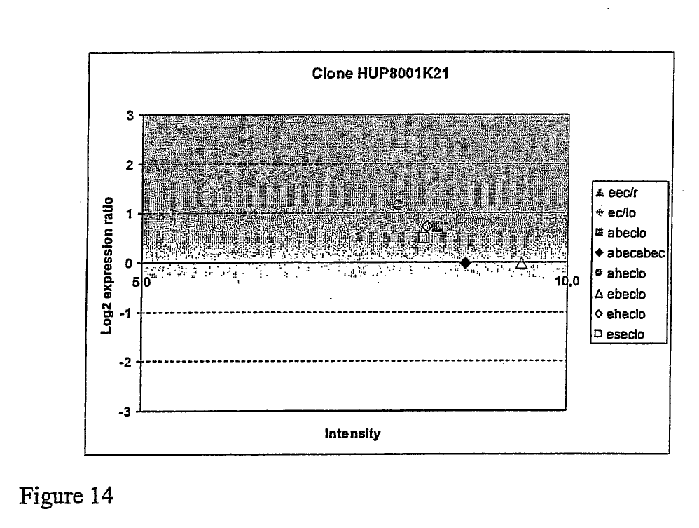

The gene having the sequence shown in SEQ ID NO:11 was identified as selectively expressed during angiogenesis based on microarray data, see FIG. 14. Specific data are given below in Table 5. Using sequence and annotation databases the equivalent gene in mice (SEQ ID NO:12) and the human homolog (SEQ ID NO:14) was also deduced. Proteins encoded by these sequences are given at SEQ ID NO:s 13 and 15, respectively.

TABLE 5
Expression profile data for gene
HUP8001K21.

| | intensity | log2 exp ratio | p-value | rank |
|---|---|---|---|---|
| eec/r | 8.5 | 0.88 | | 594 |
| ec/lo | 8.4 | 0.80 | | 442 |
| abeclo | 8.5 | 0.71 | 1.00 | |
| abecebec | 8.8 | −0.02 | 0.48 | |
| aheclo | 8.0 | 1.15 | 1.00 | 15 |
| ebeclo | 9.5 | −0.03 | 0.47 | |
| eheclo | 8.3 | 0.72 | 0.98 | |
| eseclo | 8.3 | 0.49 | 0.99 | |

Based on this expression profile, the gene was further analyzed in zebrafish embryos. Two corresponding zebrafish genes were identified for targeting. Two morpholinos were prepared, sz257 and sz258, each targeted to one of the zebrafish genes. Twelve (12) ng of sz257 morpholino and 12 ng of sz258 morpholino were administered to each fertilized egg. The embryos were allowed to develop. At 24 hpf a secondary in situ hybridization screen with six different probes was conducted.

Intersegmental expression was analyzed in the assay and results differed somewhat based on the probe used. The probe fli-1 revealed that all of the 15 morphant embryos assayed were normal. The probe flk-1 indicated that 13% of the 16 morphants reviewed had low effects and 6% had high effects. When analyzed through the probe tie-1, all 15 morphants observed were normal. The probe cdh5 indicated that all 26 morphants observed were normal. The probe flt-4 indicated that all 17 morphants observed were normal, the probe tie-2 showed all 20 observed morphants as normal.

The following table, Table 6, summarizes this data.

TABLE 6
secondary in situ hybridization data

| Probe | Number analyzed | Results |
|---|---|---|
| fli-1 | 15 | normal |
| flk-1 | 16 | 13% L, 6% H |
| tie-1 | 15 | normal |
| cdh5 | 26 | normal |
| flt-4 | 17 | normal |
| tie-2 | 20 | normal |

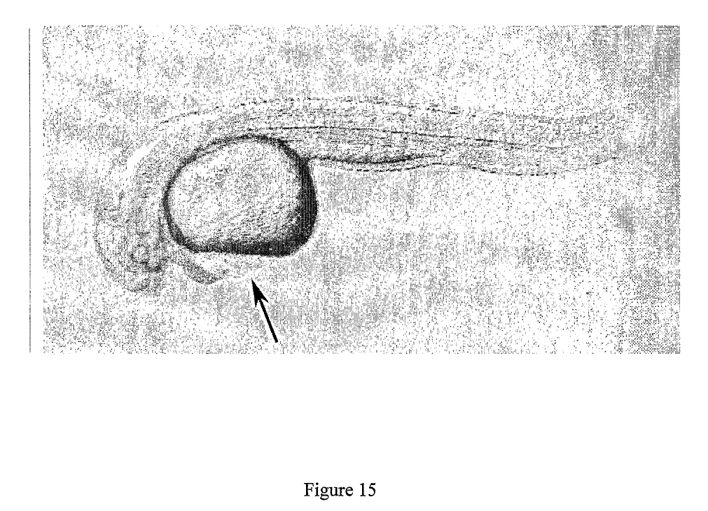

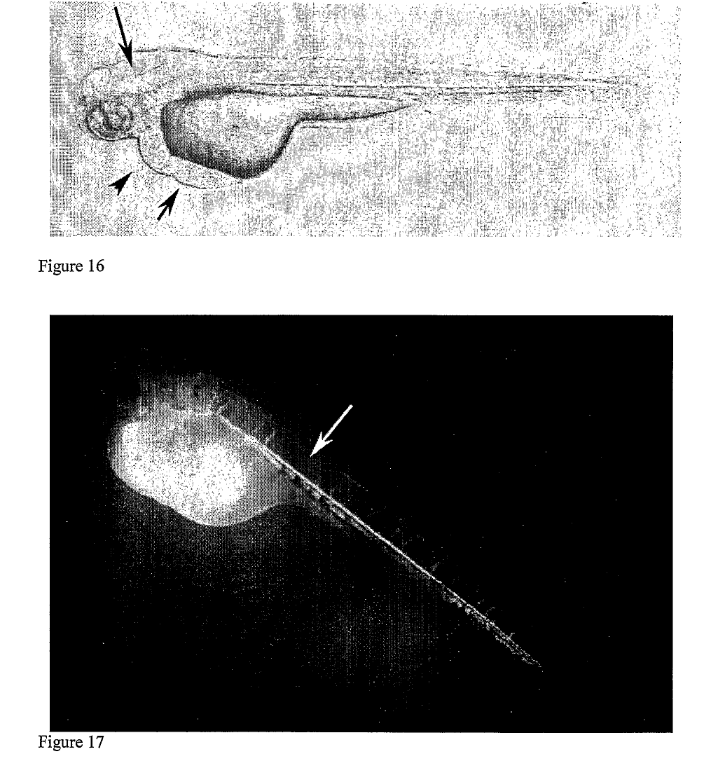

At 28 hpf embryos were observed morphologically. FIG. 15 shows a representative morphant embryo at 28 hpf. As indicated by the arrow, yolk sac edema was observed in 47% of the 55 morphants analyzed. At 56 hpf a total of 53 embryos were observed, a representative morphant embryo is shown in FIG. 16. As highlighted by the long arrow, expanded hindbrain was found in 34% of embryos. Yolk sac edema, shown by a short arrow, was also observed in 58% of embryos. An arrowhead points out the location checked for pericardial edema; it was not observed.

Additional analyses were conducted on 48-56 hpf morphant embryos. A primary in situ hybridization screen with cdh5 on 20 morphants showed all as normal. Microangiography on 31 morphants was used to locate the presence of FITC-Dextran in various regions of the embryo, see FIG. 17. Of the 31 embryos, 19% had FITC-Dextran in the heart but none had it in the heart and the head. Reduced intersegmental vasculature was seen as indicated by the arrow in FIG. 17. High effects were observed in 19% of the embryos, medium effects in 13% and low effects in 29%. No leaky vasculature was observed. Normal embryos accounted for 19% of the sample.

HUP8003D24

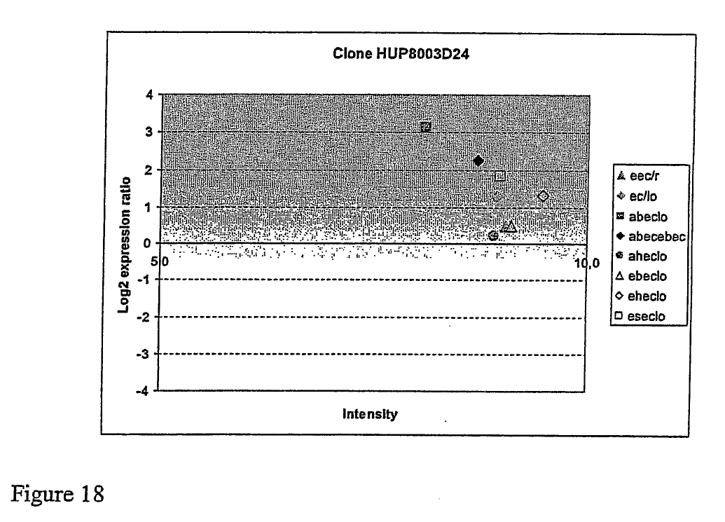

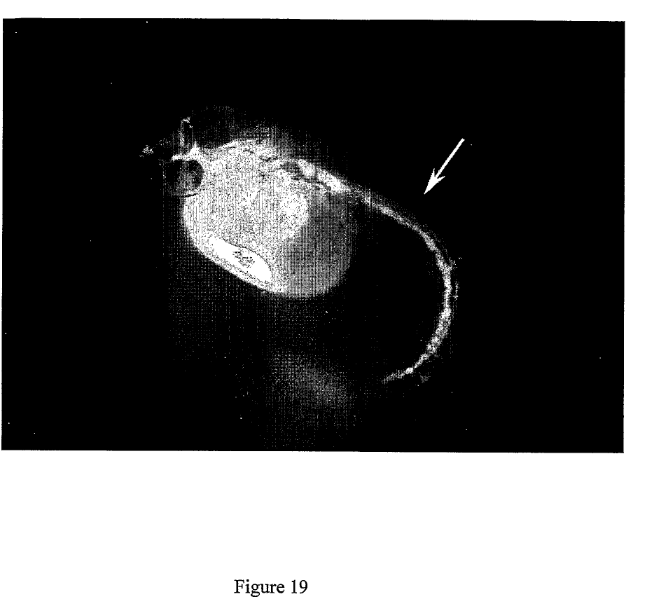

The gene having the sequence shown in SEQ ID NO:16 was identified as selectively expressed during angiogenesis based on microarray data, see FIG. 18. Specific data are given below in Table 7. Using sequence and annotation databases the equivalent gene in mice (SEQ ID NO: 17) and two human homologs (SEQ ID NO:s19 and 21) was also deduced. Proteins encoded by these sequences are given at SEQ ID NO:s 18, 20, and 22, respectively.

TABLE 7
Expression profile data for gene
HUP8003D24

| | intensity | log2 exp ratio | p-value | rank |
|---|---|---|---|---|
| eec/r | 9.1 | 0.48 | | 2061 |
| ec/lo | 9.0 | 1.30 | | 149 |
| abeclo | 8.1 | 3.14 | 1.00 | |
| abecebec | 8.7 | 2.26 | 1.00 | |
| aheclo | 8.9 | 0.23 | 0.85 | |
| ebeclo | 9.1 | 0.51 | 0.90 | |
| eheclo | 9.5 | 1.32 | 1.00 | |
| eseclo | 9.0 | 1.84 | 1.00 | |

Based on this expression profile, the gene was further analyzed in zebrafish embryos. Three corresponding zebrafish genes were identified for targeting. Two morpholinos were prepared, sz185 and sz186, which were targeted to the three zebrafish genes. For the lower dose group, 3 ng of sz185 morpholino and 6 ng of sz186 morpholino were administered to each fertilized egg. For the double dose group, 6 ng of sz185 morpholino and 12 ng of sz186 morpholino were administered. The embryos were allowed to develop. At 24 hpf a secondary in situ hybridization screen with six different probes was conducted.

Intersegmental expression was analyzed in the assay and results differed somewhat based on the probe used. In the lower dose group, the probe fli-1 revealed that 18% of the 11 morphant embryos assayed had medium effects, i.e., 36-70% loss of intersegmental expression, and 18% had high effects. The probe flk-1 indicated that 36% of the 11 morphants reviewed had high effects. The probe tie-1 indicated that 29% of the 14 morphants observed had high effects. The probe cdh5 indicated 31% of the 16 morphants observed had high effects, and 31% had medium effects. The probe flt-4 indicated that all 16 morphants observed were normal, and the probe tie-2 indicated that all 15 morphants observed were normal.

In the higher dose group, the probe fli-1 revealed that 33% of the 3 morphant embryos assayed had low effects and 33% had medium effects. The probe flk-1 indicated that 100%, or both of the 2 morphants reviewed, had medium effects. The probe cdh5 indicated 100%, all 7 of the morphants observed had high effects. The probe flt-4 indicated that all 3 morphants observed were normal, and the probe tie-2 indicated that all 7 morphants observed were normal.

The following Table 8 summarizes the foregoing data.

TABLE 8
secondary in situ hybridization data

| | Morphants with | | Morphants with | |
|---|---|---|---|---|
| | 3 ng sz185, | | 6 ng sz185, | |
| | 6 ng sz186 | | 12 ng sz186 | |
| | Number | | Number | |
| Probe | analyzed | Results | analyzed | Results |
| fli-1 | 11 | 18% M, 36% H | 3 | 33% L, 33% M |
| flk-1 | 11 | 36% H | 2 | 100% M |
| tie-1 | 14 | 29% H | n/a | no data |
| cdh5 | 16 | 31% M, 31% H | 7 | 100% H |
| flt-4 | 16 | normal | 3 | normal |
| tie-2 | 15 | normal | 7 | normal |

At 28 hpf embryos were observed morphologically. Cell death was observed in 70% of the 66 embryos observed. Yolk sac edema was observed in 29% of the morphants. At 56 hpf a total of 66 embryos were observed for phenotypic characteristics. Yolk sac edema was observed in 42% of embryos, 35% showed reduced IS blood flow and 26% showed reduced blood flow.

Additional analyses were conducted on 48-56 hpf morphant embryos.

A primary in situ hybridization screen with cdh5 on 21 morphants receiving the lower doses noted above (3ng sz185, 6 ng sz86) showed 52% as normal. Low effects of reduced intersegmental expression were seen in 43% of the embryos, and medium effects in 5%. Embryos receiving the double doses (6 ng sz185, 12 ng sz186), when viewed at the 48-56 hpf stage revealed 74% of the that the 23 embryos observed were normal. Low effects of reduced intersegmental expression were observed in 22% and medium effects in 4% of the embryos.

Microangiography on morphants given the lower dose of morpholinos (3 ng sz185, 6 ng sz186) was used to locate the presence of FITC-Dextran in various regions of the embryo, see FIG. 19. Of the 33 embryos, none had FITC-Dextran in the heart, or the heart and head. Reduced intersegmental vasculature was seen as indicated by the arrow in FIG. 19. High effects were observed in 6% of the embryos, medium effects in 15% and low effects in 36%. No leaky vasculature was observed. Normal embryos accounted for 43% of the sample.

The data reveal that the gene is expressed in many locations, such as vessels and epithelial structures in the kidneys as well as in large vessels, megakaryocytes, in heart valves and in the skin epithelium.

Gene HUP8004N1

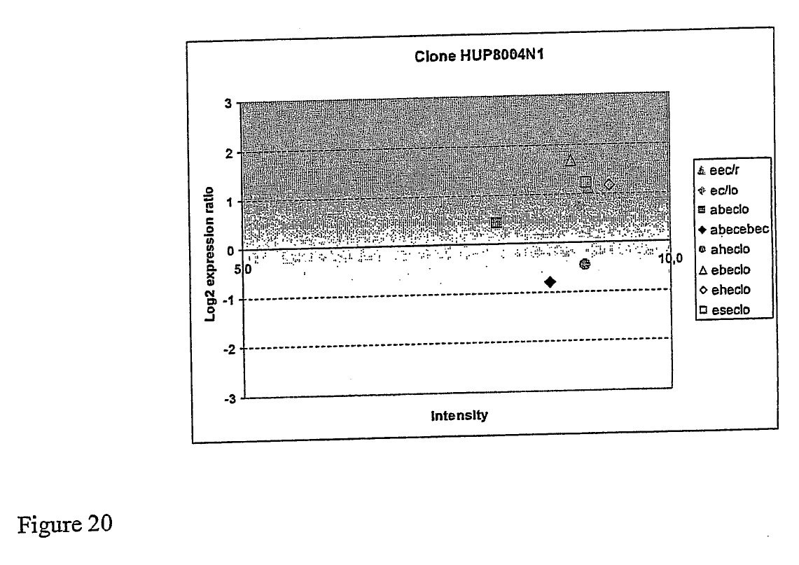

The gene having the sequence shown in SEQ ID NO:23 was identified as selectively expressed during angiogenesis based on microarray data, see FIG. 20. Specific data are given below in Table 9. Using sequence and annotation databases the equivalent gene in mice (SEQ ID NO:24) and the human homolog (SEQ ID NO:26) was also deduced. Proteins encoded by these sequences are given at SEQ ID NO:s 25 and 27, respectively.

TABLE 9
Expression profile data for gene
HUP8004N1

| | intensity | log2 exp ratio | p-value | rank |
|---|---|---|---|---|
| eec/r | 9.0 | 1.12 | | 262 |
| ec/lo | 9.0 | 0.75 | | 516 |
| abeclo | 8.0 | 0.44 | 0.79 | |
| abecebec | 8.6 | −0.80 | 0.03 | |
| aheclo | 9.0 | −0.47 | 0.02 | |
| ebeclo | 8.8 | 1.68 | 1.00 | |
| eheclo | 9.3 | 1.18 | 1.00 | |
| eseclo | 9.0 | 1.22 | 1.00 | |

Based on this expression profile, the gene was further analyzed in zebrafish embryos. Two corresponding zebrafish genes were identified for targeting. Two morpholinos were prepared, sz223 and sz224, each targeted to one of the zebrafish genes. Two dosing strategies were employed. The first dose group received 2 ng of sz223 morpholino and 1 ng of sz224 morpholino in each fertilized egg. The second dose group received 1 ng of sz223 morpholino and 0.5 ng of sz224 morpholino in each fertilized egg. The embryos were allowed to develop. At 24 hpf a secondary in situ hybridization screen with six different probes was conducted.

Intersegmental expression in embryos from the second dose group (1 ng sz223, 0.5 ng sz224) was analyzed in the assay and results differed somewhat based on the probe used. The probe fli-1 revealed that 7% of the 15 morphant embryos assayed had low effects, and 7% had high effects. The probe flk-1 indicated that 7% of the 14 morphants reviewed had high effects. The probe tie-1 indicated that 7% of the 14 morphants observed had low effects, and 7% had high effects. The probe cdh5 indicated that 8% of the 26 morphants observed had medium effects. The probe flt-4 indicated that all 15 morphants observed were normal, and the probe tie-2 indicated that all 15 morphants observed were normal.

The following table, Table 10, summarizes this data.

TABLE 10
secondary in situ hybridization data

| Probe | Number analyzed | Results |
|---|---|---|
| fli-1 | 15 | 7% L, 7% H |
| flk-1 | 14 | 7% H |
| tie-1 | 14 | 7% L, 7% H |
| cdh5 | 26 | 8% M |
| flt-4 | 15 | normal |
| tie-2 | 15 | normal |

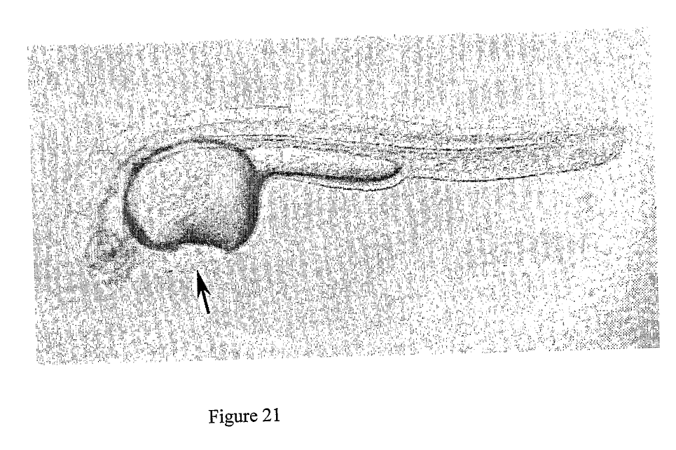

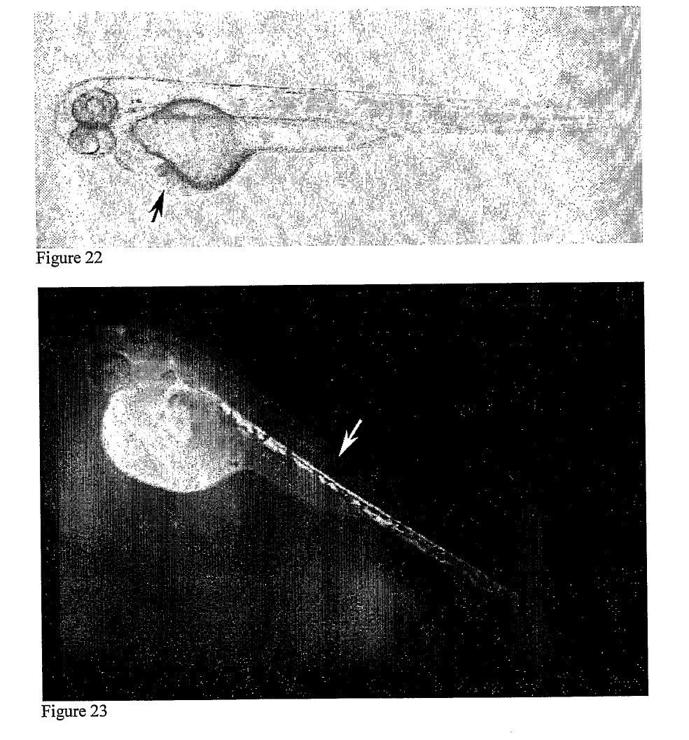

At 28 hpf embryos were observed morphologically. FIG. 21 shows a representative morphant embryo at 28 hpf. As indicated by the arrow, yolk sac edema was observed in 56% of the 59 morphants studied. At 56 hpf a total of 20 embryos were observed for phenotypic characteristics, a representative morphant embryo is shown in FIG. 22. As indicated by the arrow, pericardial edema was observed in 35% of embryos, 65% had a blood pool in the yolk, also indicated by the arrow, and 30% showed reduced IS blood flow.

Additional analyses were conducted on 48-56 hpf morphant embryos from the second dose group. A primary in situ hybridization screen with cdhS on 22 morphants showed all as normal. Microangiography on 30 second dose group morphants was used to locate the presence of FITC-Dextran in various regions of the embryo, see FIG. 23. Of the 30 embryos, 13% had FITC-Dextran in the heart, and 3% in the heart and head. Reduced intersegmental vasculature was seen as indicated by the arrow in FIG. 23. High effects were observed in 7% of the embryos and low effects in 20%. No leaky vasculature was observed. Normal embryos accounted for 57% of the sample.

The data reveal that the gene is expressed in specific endothelium. In kidneys, it is expressed by certain vessels and some other epithelial structures. There is also some expression in the liver.

Gene HUP8010A10

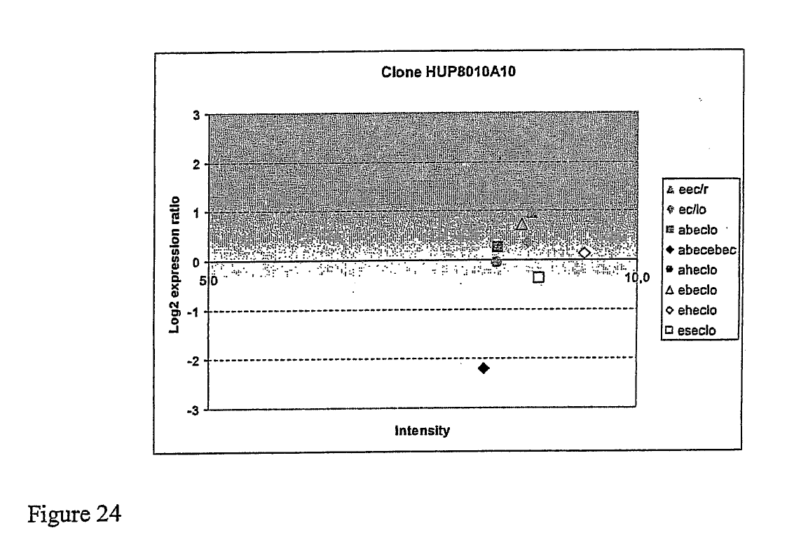

The gene having the sequence shown in SEQ ID NO:28 was identified as selectively expressed during angiogenesis based on microarray data, see FIG. 24. Specific data are given below in Table 11. Using sequence and annotation databases the equivalent gene in mice (SEQ ID NO:29) and the human homolog (SEQ ID NO:31) was also deduced. Proteins encoded by these sequences are given at SEQ ID NO:s 30 and 32, respectively.

TABLE 11
Expression profile data for gene
HUP8010A10

| | intensity | log2 exp ratio | p-value | rank |
|---|---|---|---|---|
| eec/r | 8.8 | 0.98 | | 424 |
| ec/lo | 8.7 | 0.36 | | 1747 |
| abeclo | 8.4 | 0.26 | 0.63 | |
| abecebec | 8.2 | −2.20 | 0.00 | |
| aheclo | 8.4 | −0.06 | 0.38 | |
| ebeclo | 8.7 | 0.72 | 0.95 | |
| eheclo | 9.4 | 0.14 | 0.67 | |
| eseclo | 8.9 | −0.37 | 0.03 | |

Based on this expression profile, the gene was further analyzed in zebrafish embryos. A corresponding zebrafish genes was identified for targeting. Two morpholinos were prepared, sz267 and sz268, each targeted to one of the zebrafish genes. In a first dosage group, 4 ng of sz267 morpholino and 2 ng of sz268 morpholino were administered to each fertilized egg. In a second dosage group, 6ng of sz267 morpholino and 3 ng of sz268 morpholino were administered. The embryos were allowed to develop. At 24 hpf a secondary in situ hybridization screen with six different probes was conducted.

Intersegmental expression was analyzed in the assay and results differed somewhat based on the probe used. In the first dose group (4 ng sz267, 2 ng sz268), the probe fli-1 revealed that 13% of the 15 morphant embryos assayed had high effects. The probe flk-1 indicated that 33% of the 15 morphants reviewed had low effects and 20% had high effects. The probe tie-1 indicated that all 17 morphants observed were normal. The probe cdh5 indicated that 8% of the 25 morphants observed had high effects. The probe flt-4 indicated that all 13 morphants observed were normal, and the probe tie-2 indicated that all 16 morphants observed were normal.

In the second dose group (6 ng sz267, 3 ng sz268), the probe fli-1 revealed that 25% of the 16 morphant embryos assayed had low effects, and 19% had high effects. The probe flk-1 indicated that 33% of the 6 morphants reviewed had low effects. The probe tie-1 indicated that 67% of the 15 morphants observed had high effects. The probe cdh5 indicated that 21% of the 24 morphants observed had low effects, 13% had medium effects, and 29% had high effects. The probe flt-4 indicated that all 15 morphants observed were normal, and the probe tie-2 indicated that all 15 morphants observed were normal.

The following Table 12 summarizes the foregoing data.

TABLE 12
secondary in situ hybridization data

| | Morphants with | | Morphants with | |
|---|---|---|---|---|
| | 4 ng sz267, | | 6 ng sz267, | |
| | 2 ng sz268 | | 3 ng sz268 | |
| | Number | | Number | |
| Probe | analyzed | Results | analyzed | Results |
| fli-1 | 15 | 13% H | 16 | 25% L, 19% H |
| flk-1 | 15 | 33% L, 20% H | 6 | 33% L |
| tie-1 | 17 | normal | 15 | 67% H |
| cdh5 | 25 | 8% H | 24 | 21% L, 13% M, |
| | | | | 29% H |
| flt-4 | 13 | normal | 15 | normal |
| tie-2 | 16 | normal | 15 | normal |

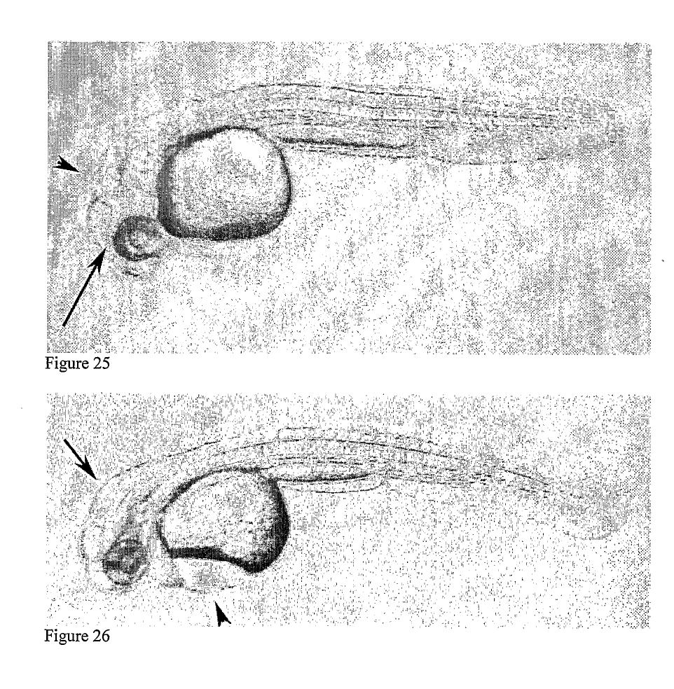

At 28 hpf embryos were observed morphologically. FIG. 25 shows a representative morphant embryo at 28 hpf. Cell death in the head was observed in 47% of the 61 embryos observed, as indicated by the arrow in FIG. 25. The arrowhead indicates expanded hindbrain, which was seen in 51% of embryos. Mild yolk sac edema was observed in 21% of the morphants. At 56 hpf a total of 59 embryos were observed for phenotypic characteristics, a representative morphant embryo is shown in FIG. 26. The arrow indicates expanded hindbrain, which was seen in 44% of embryos. Mild yolk sac edema was observed in 29% of the morphants and is indicated by the arrowhead. Reduced IS blood flow was noted in 14% of the embryos, and reduced blood flow was found in 17% of the embryos.

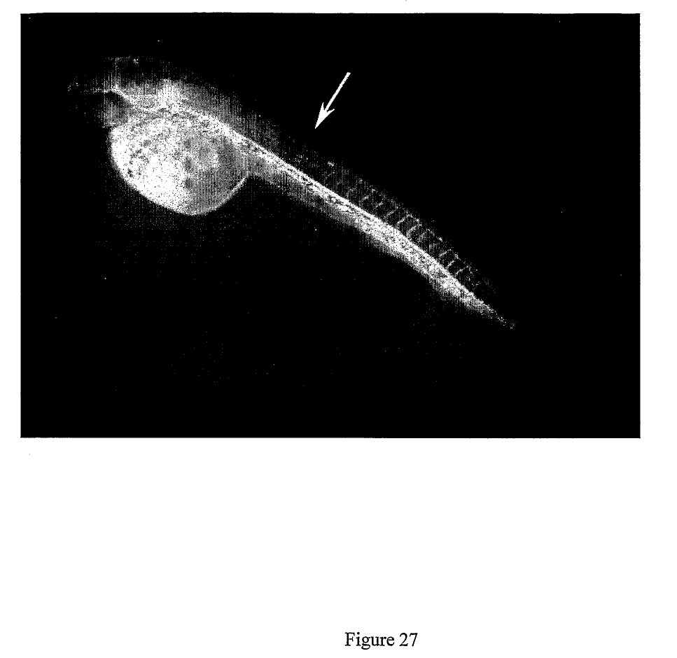

Additional analyses were conducted on 48-56 hpf morphant embryos from the second dose group. A primary in situ hybridization screen with cdh5 on 19 morphants showed 68% as normal. Low effects of reduced intersegmental expression were seen in 32% of the embryos. Microangiography on morphants was used to locate the presence of FITC-Dextran in various regions of the embryo, see FIG. 27. Of the 32 embryos, none had FITC-Dextran in the heart, or the heart and head. Reduced intersegmental vasculature was seen as indicated by the arrow in FIG. 27. High effects were observed in 6% of the embryos, medium effects in 3% and low effects in 34%. No leaky vasculature was observed. Normal embryos accounted for 56% of the sample.

Gene NOC8003L17

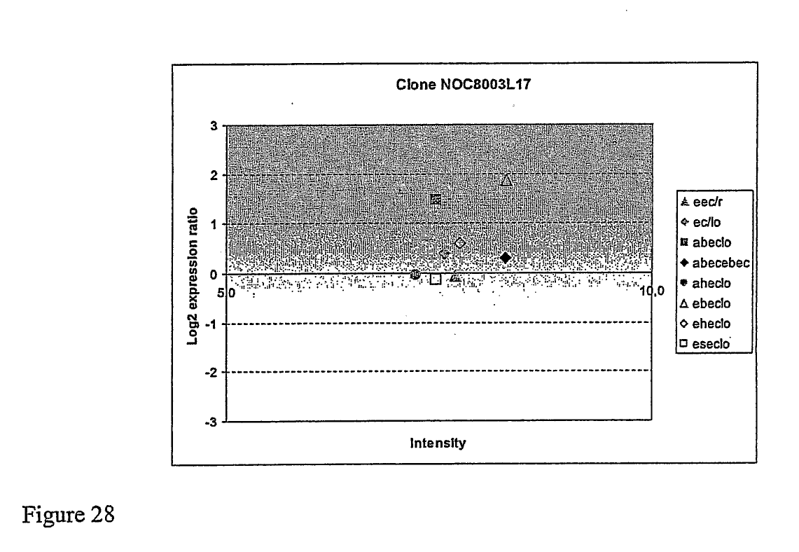

The gene having the sequence shown in SEQ ID NO:33 was identified as selectively expressed in blood vessels based on microarray data, see FIG. 28. Specific data are given below in Table 13. Using sequence and annotation databases the equivalent gene in mice (SEQ ID NO:34) and the human homolog (SEQ ID NO:36) was also deduced. Proteins encoded by these sequences are given at SEQ ID NO:s 35 and 37, respectively.

TABLE 13
Expression profile data for gene
NOC8003L17

| | intensity | log2 exp ratio | p-value | rank |
|---|---|---|---|---|
| eec/r | 7.7 | −0.06 | | 6087 |
| ec/lo | 7.6 | 0.40 | | 1547 |
| abeclo | 7.5 | 1.47 | 0.97 | |
| abecebec | 8.3 | 0.30 | 0.77 | |
| aheclo | 7.2 | −0.04 | 0.41 | |
| ebeclo | 8.3 | 1.87 | 1.00 | |
| eheclo | 7.8 | 0.60 | 0.99 | |
| eseclo | 7.5 | −0.13 | 0.24 | |

Based on this expression profile, the gene was further analyzed in zebrafish embryos. One corresponding zebrafish gene was identified for targeting. Two morpholinos were prepared, sz180 and sz181, each targeted to the zebrafish gene. In a first dose group, 12 ng of sz180 and 1 ng of sz181 were administered to each fertilized egg. In a second dose group, 12 ng of sz189 and 2 ng of sz181 were administered to each fertilized egg. The embryos were allowed to develop. At 24 hpf a secondary in situ hybridization screen with six different probes was conducted.

Intersegmental expression in embryos from the first dose group was analyzed in the assay and results differed somewhat based on the probe used. The probe fli-1 revealed that 40 of the 15 morphant embryos assayed had high effects. The probe flk-1 indicated that all 7 of the morphants reviewed were normal. The probe tie-1 indicated that all 15 morphants observed were normal. The probe cdh5 indicated that 15% of the 20 morphants observed had low effects, as well as 5% with medium effects and 30% with high effects. The probe flt-4 indicated that all 11 morphants observed were normal, and the probe tie-2 indicated that all 16 morphants observed were normal.

The following table, Table 14, summarizes this data.

TABLE 14
secondary in situ hybridization data

| Probe | Number analyzed | Results |
|---|---|---|
| fli-1 | 15 | 40% H |
| flk-1 | 7 | normal |
| tie-1 | 15 | normal |
| cdh5 | 20 | 15% L, 5% M, 30% H |
| flt-4 | 11 | normal |
| tie-2 | 16 | normal |

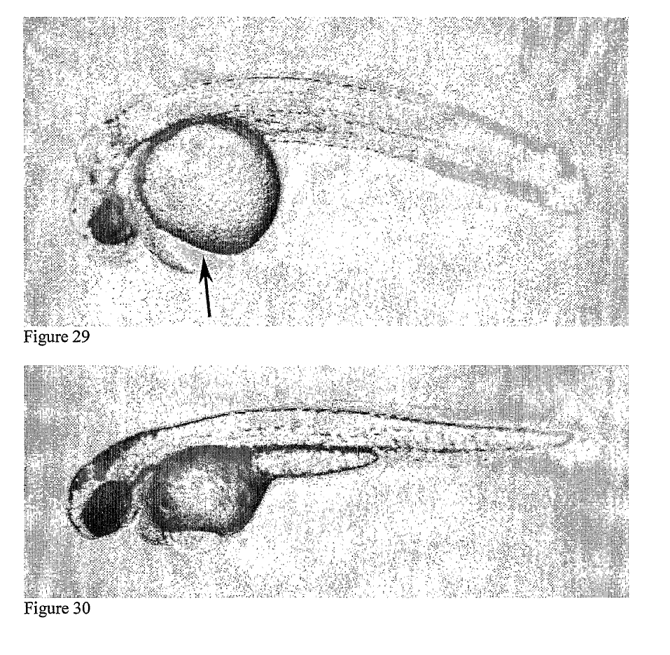

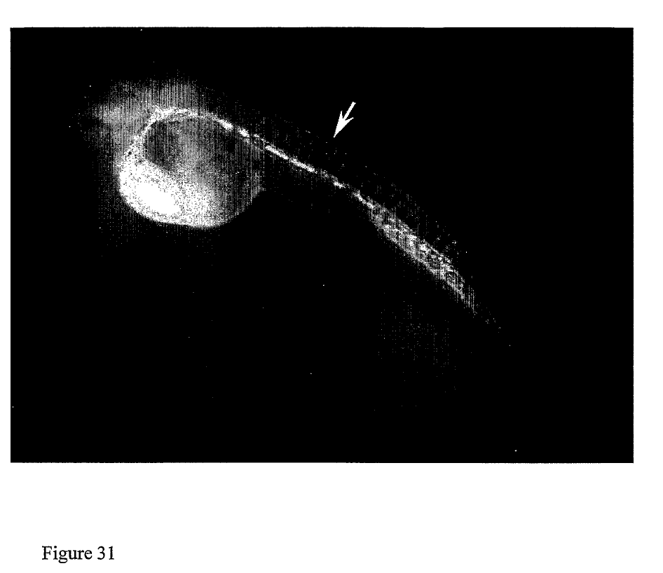

At 28 hpf embryos were observed morphologically. FIG. 29 shows a representative morphant embryo. As indicated by the arrow, yolk sac edema was observed in 67% of the 48 morphants studied. Embryos observed at 56 hpf demonstrated normal morphology, a representative embryo is shown in FIG. 30.

Additional analyses were conducted on 48-56 hpf morphant embryos. A primary in situ hybridization screen with cdh5 on 14 morphants from the second dose group showed 79% as normal, the remaining 29% showing medium effects of reduced intersegmental expression. Microangiography on 29 first dose group morphants was used to locate the presence of FITC-Dextran in various regions of the embryo, see FIG. 31. Of the 29 embryos, none had FITC-Dextran in the heart, 3% had FITC-Dextran in the heart and head. Reduced intersegmental vasculature was seen as indicated by the arrow in FIG. 31. High effects were observed in 7% of the embryos, medium effects in 3% and low effects in 38%. No leaky vasculature was observed. Normal embryos accounted for 45% of the sample.

GeneNOC8009C9

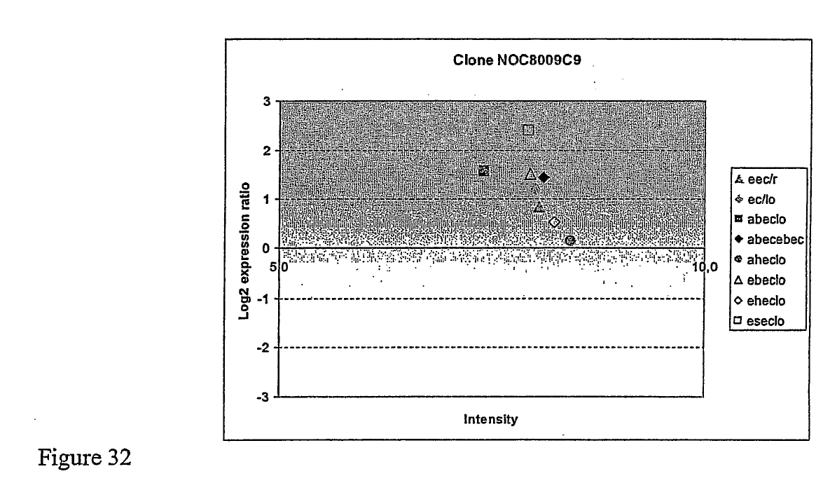

The gene having the sequence shown in SEQ ID NO:38 was identified as selectively expressed during angiogenesis based on microarray data, see FIG. 32. Specific data are given below in Table 15. Using sequence and annotation databases the equivalent gene in mice (SEQ ID NO:39) and the human homolog (SEQ ID NO:41) was also deduced. Proteins encoded by these sequences are given at SEQ ID NO:s 40 and 42, respectively.

TABLE 15
Expression profile data for gene
NOC8009C9

| | intensity | log2 exp ratio | p-value | rank |
|---|---|---|---|---|
| eec/r | 8.1 | 0.84 | | 685 |
| ec/lo | 8.0 | 1.19 | | 184 |
| abeclo | 7.4 | 1.57 | 1.00 | |
| abecebec | 8.1 | 1.44 | 1.00 | |
| aheclo | 8.4 | 0.12 | 0.74 | |
| ebeclo | 8.0 | 1.52 | 1.00 | |
| eheclo | 8.3 | 0.53 | 0.98 | |
| eseclo | 7.9 | 2.41 | 1.00 | |

Based on this expression profile, the gene was further analyzed in zebrafish embryos. One corresponding zebrafish genes was identified for targeting. Two morpholinos were prepared, sz241 and sz242, targeted to the zebrafish gene. Three (3)ng of sz241 morpholino and 1 ng of sz242 morpholino were administered to each fertilized egg. The embryos were allowed to develop. At 24 hpf a secondary in situ hybridization screen with six different probes was conducted.

Intersegmental expression was analyzed in the assay and results differed somewhat based on the probe used. The probe fli-1 revealed that 7% of the 15 morphant embryos assayed had medium effects. The probe flk-1 indicated that all 15 morphants observed were normal. The probe tie-1 indicated that 7% of the 15 morphants observed had high effects. The probe cdh5 indicated that 15% of the 20 morphants observed had low effects. Medium effects were seen in 5% and high effects in 15%. The probe flt-4 indicated that all 15 morphants observed were normal, and the probe tie-2 indicated that all 16 morphants observed were normal.

The following table, Table 16, summarizes this data.

TABLE 16
secondary in situ hybridization data

| Probe | Number analyzed | Results |
|---|---|---|
| fli-1 | 15 | 7% M |
| flk-1 | 15 | normal |
| tie-1 | 15 | 7% H |
| cdh5 | 20 | 15% L, 5% M, 15% H |
| flt-4 | 15 | normal |
| tie-2 | 16 | normal |

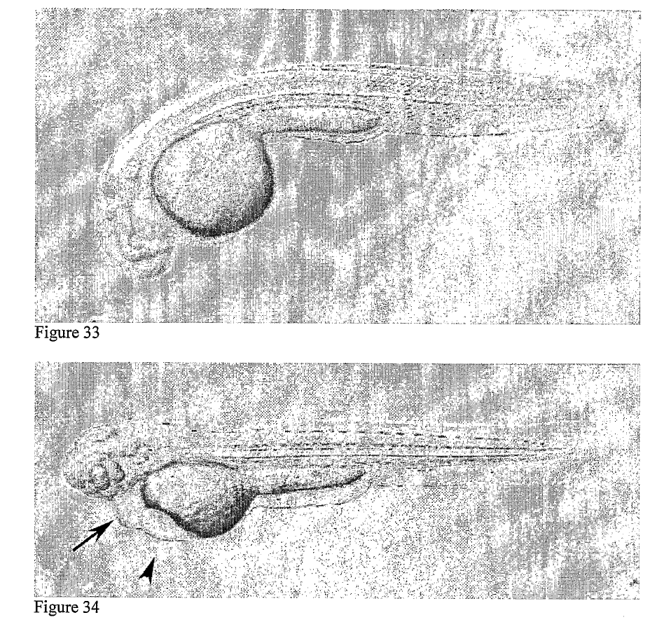

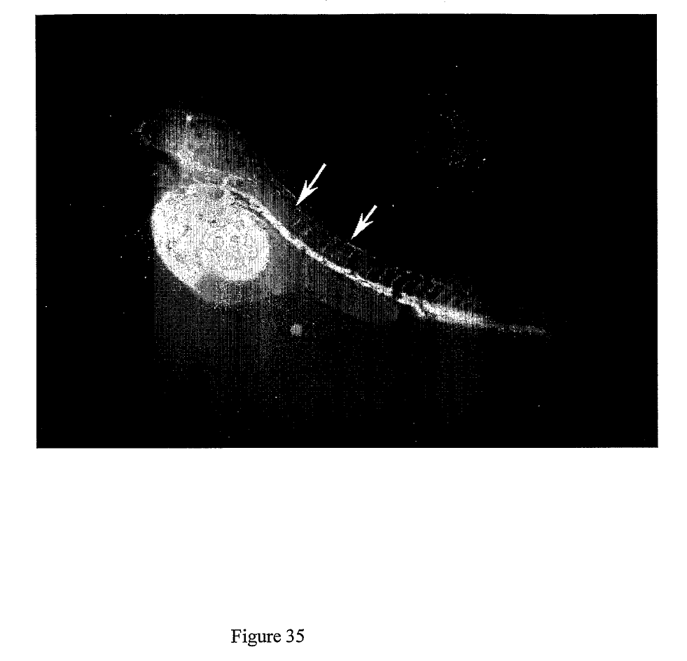

At 28 hpf embryos were observed morphologically. FIG. 33 shows a representative morphant embryo at 28 hpf. Mild cell death was observed in 25% of the 53 embryos observed. At 56 hpf 52 embryos were observed for phenotypic characteristics, a representative morphant embryo is shown in FIG. 34. As indicated by the arrow, pericardial edema was seen in 13% of embryos. The arrowhead points toward a region of yolk sac edema, seen in 25% of embryos. Reduced IS blood flow was observed in 13% and 15% showed reduced axial blood flow.

Additional analyses were conducted on 48-56 hpf morphant embryos. A primary in situ hybridization screen with cdh5 on 31 morphants showed all as normal. Microangiography on morphants was used to locate the presence of FITC-Dextran in various regions of the embryo, see FIG. 35. Of the 29 embryos, 7% had FITC-Dextran in the heart, and 14% in the heart and head. Reduced intersegmental vasculature was seen as indicated by the arrows in FIG. 35. High effects were observed in 3% of the embryos, medium effects in 3% and low effects in 21%. No leaky vasculature was observed. Normal embryos accounted for 52% of the sample. The data reveal that the gene is expressed in and around the heart and around organs, including some expression in select organs.

Gene NOC8009G23

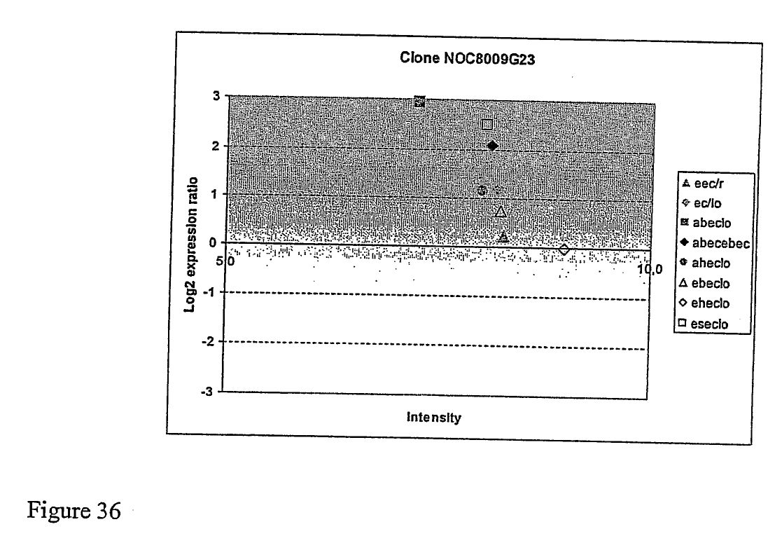

The gene having the sequence shown in SEQ ID NO:43 was identified as selectively expressed during angiogenesis based on microarray data, see FIG. 36. Specific data are given below in Table 17. Using sequence and annotation databases the equivalent gene in mice (SEQ ID NO:44) and the human homolog (SEQ ID NO:46) was also deduced. Proteins encoded by these sequences are given at SEQ ID NO:s 45 and 47, respectively.

TABLE 17
Expression profile data for gene
NOC8009G23

| | intensity | log2 exp ratio | p-value | rank |
|---|---|---|---|---|
| eec/r | 8.3 | 0.23 | | 3262 |
| ec/lo | 8.2 | 1.19 | | 185 |
| abeclo | 7.2 | 2.94 | 1.00 | |
| abecebec | 8.1 | 2.09 | 1.00 | |
| aheclo | 8.0 | 1.15 | 1.00 | |
| ebeclo | 8.2 | 0.75 | 0.94 | |
| eheclo | 9.0 | −0.02 | 0.47 | |
| eseclo | 8.1 | 2.51 | 1.00 | |

Based on this expression profile, the gene was further analyzed in zebrafish embryos. One corresponding zebrafish gene was identified for targeting. Two morpholinos were prepared, sz149 and sz150, each targeted to the zebrafish gene. In a first dose group, 1.5 ng of sz149 morpholino and 1.5 ng of sz150 morpholino were administered to fertilized eggs. In a second dose group, 2 ng of sz149 morpholino and 2 ng of sz150 morpholino were administered to fertilized eggs. In a third dose group, 3 ng of sz149 morpholino and 3 ng of sz150 morpholino were administered to fertilized eggs. The embryos were allowed to develop. At 24 hpf a secondary in situ hybridization screen with six different probes was conducted.

Intersegmental expression was analyzed in the assay and results differed somewhat based on the probe used. In studies with embryos from the second dose group, the probe fli-1 revealed that 21% of the 14 morphant embryos assayed had high effects. The probe flk-1 indicated that all 16 morphants reviewed were normal. The probe tie-1 indicated that 36% of the 22 morphants observed had high effects. The probe cdh5 indicated that 13% of the 15 morphants observed had low effects with breaks in axial expression. The probe flt-4 indicated that all 16 morphants observed were normal, and the probe tie-2 indicated that all 13 morphants observed were normal.

In studies from the second dose group, the probe fli-1 revealed that 13% of the 15 morphant embryos assayed had medium effects, 27% had high effects. The probe flk-1 indicated that 7% of the 15 morphants reviewed had low effects, 7% had medium effects and 20% had high effects. The probe tie-1 indicated that 62% of the 13 morphants observed had high effects. The probe cdh5 indicated that 25% of the 12 morphants observed had high effects, some with breaks in axial expression. The probe flt-4 indicated that 13% of the 15 morphants observed had low effects in the axial vessels, including breaks in axial expression and severely malformed tails. The probe tie-2 indicated that all 8 morphants observed were normal.

The following Table 18 summarizes the foregoing data.

TABLE 18
secondary in situ hybridization data

| | Morphants with | | Morphants with | |
|---|---|---|---|---|
| | 2 ng sz149, | | 3 ng sz149, | |
| | 2 ng sz150 | | 3 ng sz150 | |
| | Number | | Number | |
| Probe | analyzed | Results | analyzed | Results |
| fli-1 | 14 | 21% H | 15 | 13% M, 27% H |
| flk-1 | 16 | normal | 15 | 7% L, 7% M, 20% H |
| tie-1 | 22 | 36% H | 13 | 62% H |
| cdh5 | 15 | 13% L*** | 12 | 25% H*** |
| flt-4 | 16 | normal | 15 | 13% low axial |
| | | | | effects |
| tie-2 | 13 | normal | 8 | normal |

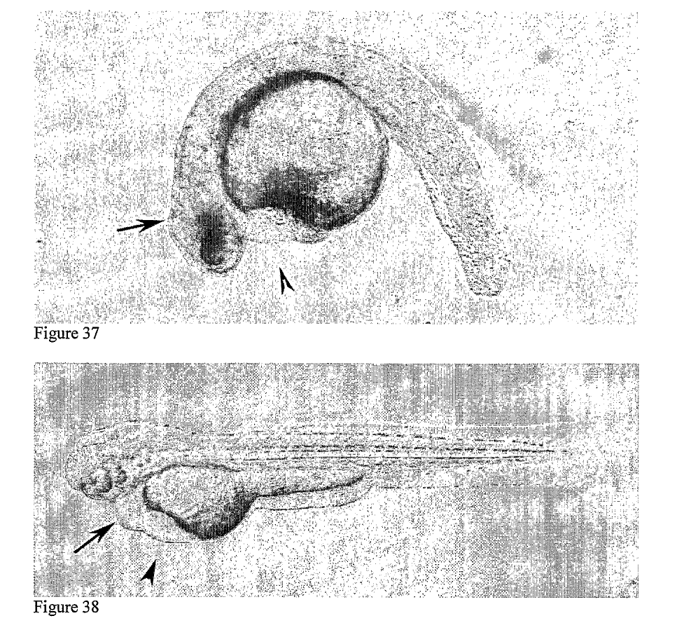

At 28 hpf embryos were observed morphologically. FIG. 37 shows a representative morphant embryo at 28 hpf. As indicated by the arrow, cell death was observed in 40% of the 20 embryos observed. As indicated by the arrowhead, yolk sac edema was observed in 55% of the morphants. Curly down body was seen in 40% of morphants. At 56 hpf a total of 20 embryos were observed for phenotypic characteristics, a representative morphant embryo is shown in FIG. 38. As shown by the arrow, pericardial edema was observed in 55% of morphants. The arrowhead points toward yolk sac edema, observed in 55% of morphants. Curly down body was reported in 30% of embryos.

Additional analyses were conducted on 48-56 hpf morphant embryos. A primary in situ hybridization screen with cdh5 on 22 morphants from the first dose group showed 90% as normal. Low effects of reduced intersegmental expression and curly down embryos were seen in 5% of the embryos, and high effects with very short tails were seen in 5% of embryos. A primary in situ hybridization screen with cdh5 was also performed on 20 morphants from the third dose group, showing 80% as normal. Low effects of reduced intersegmental expression were seen in 5% of the embryos, and medium effects in 15% of embryos.

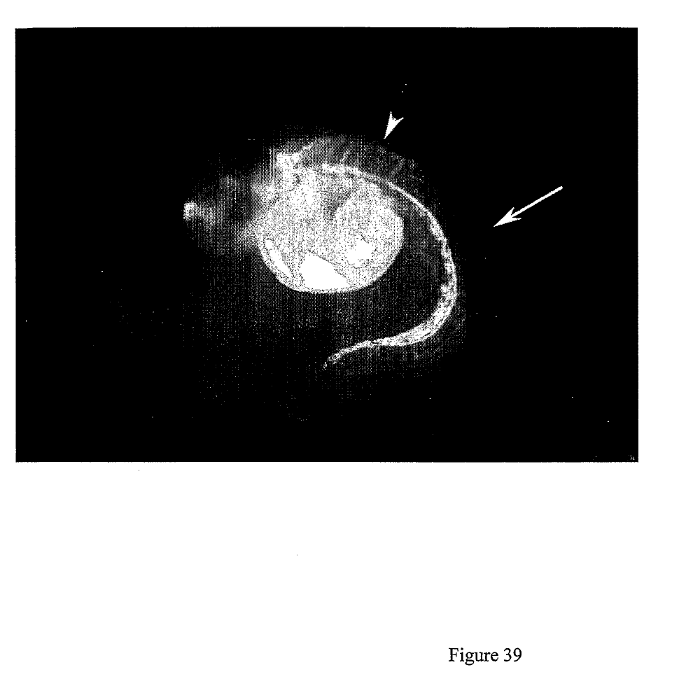

Microangiography on morphants was used to locate the presence of FITC-Dextran in various regions of the embryo, see FIG. 39. Of the 25 embryos, none had FITC-Dextran in the heart, or the heart and head. Reduced intersegmental vasculature was seen in 24% of the embryos as indicated by the arrow in FIG. 39. No leaky vasculature was observed. Normal embryos accounted for 76% of the sample, in example of normal intersegmental vessels is indicated by the arrowhead.

Gene OJC8003C9

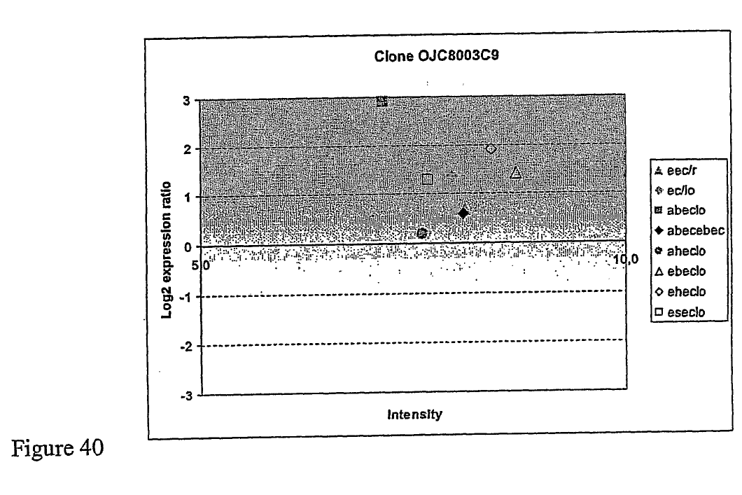

The gene having the sequence shown in SEQ ID NO:48 was identified as selectively expressed in blood vessels based on microarray data, see FIG. 40. Specific data are given below in Table 19. Using sequence and annotation databases the equivalent gene in mice (SEQ ID NO:49) and the human homolog (SEQ ID NO:51) was also deduced. Proteins encoded by these sequences are given at SEQ ID NO:s 50 and 52, respectively.

TABLE 19
Expression profile data for gene
OJC8003C9

| | intensity | log2 exp ratio | p-value | rank |
|---|---|---|---|---|
| eec/r | 8.1 | 0.67 | | 1246 |
| ec/lo | 8.0 | 1.37 | | 123 |
| abeclo | 7.1 | 2.90 | 1.00 | |
| abecebec | 8.1 | 0.59 | 0.92 | |
| aheclo | 7.6 | 0.19 | 0.85 | |
| ebeclo | 8.7 | 1.41 | 1.00 | |
| eheclo | 8.4 | 1.89 | 1.00 | |
| eseclo | 7.7 | 1.28 | 1.00 | |

on this expression profile, the gene was further analyzed in zebrafish embryos. corresponding zebrafish gene was identified for targeting. Two morpholinos were prepared, sz129 and sz130, each targeted to the zebrafish gene. In a first dose group, 3 ng of sz129 morpholino and 4.5 ng of sz130 morpholino were administered to each fertilized egg. In a second dose group, 4 ng of sz129 morpholino and 6 ng of sz130 morpholino were administered to each fertilized egg. In a third dose group, 6 ng of sz129 morpholino and 8 ng of sz130 morpholino were administered to each fertilized egg. In a fourth dose group, 6 ng of sz129 morpholino and 9 ng of sz130 morpholino were administered to each fertilized egg. The embryos were allowed to develop. At 24 hpf a secondary in situ hybridization screen with six different probes was conducted.

Intersegmental expression was analyzed in the assay and results differed somewhat based on the probe used. In embryos from the second dose group, the probe fli-1 revealed that 20% of the 10 morphant embryos assayed had low effects, 10% had medium effects and 10% had high effects. The probe flk-1 indicated that 11% of the 9 morphants reviewed had low effects, 33% had medium effects and 11% had high effects. The probe tie-1 indicated that 22% of the 9 morphants observed had high effects. The probe cdh5 indicated that 14% of the 7 morphants observed had medium effects and 14% had high effects. The probe flt-4 indicated that all 9 morphants observed were normal, and the probe tie-2 indicated that all 7 morphants observed were normal.

In embryos from the third dose group, the probe fli-1 revealed that 10% of the 10 morphant embryos assayed had medium effects, and 50% had high effects. The probe flk-1 indicated that 25% of the 12 morphants reviewed had medium effects and 25% had high effects. The probe tie-1 indicated that 17% of the 6 morphants observed had low effects, and 50% had high effects. The probe cdh5 indicated that 40% of 5 morphants observed had medium effects. The probe flt-4 indicated that all 9 morphants observed were normal, and the probe tie-2 indicated that all 6morphants observed were normal.

The following Table 20 summarizes the foregoing data.

TABLE 20
secondary in situ hybridization data

| | Morphants with | | Morphants with | |
|---|---|---|---|---|
| | 3 ng sz185, | | 6 ng sz185, | |
| | 6 ng sz186 | | 12 ng sz186 | |
| | Number | | Number | |
| Probe | analyzed | Results | analyzed | Results |
| fli-1 | 10 | 20% L, 10% M, | 10 | 10% M, 50% H |
| | | 10% H | | |
| flk-1 | 9 | 11% L, 33% M, | 12 | 25% M, 25% H |
| | | 11% H | | |
| tie-1 | 9 | 22% H | 6 | 17% L, 50% H |
| cdh5 | 7 | 14% M, 14% H | 5 | 40% M |
| flt-4 | 9 | normal | 9 | normal |
| tie-2 | 7 | normal | 6 | normal |

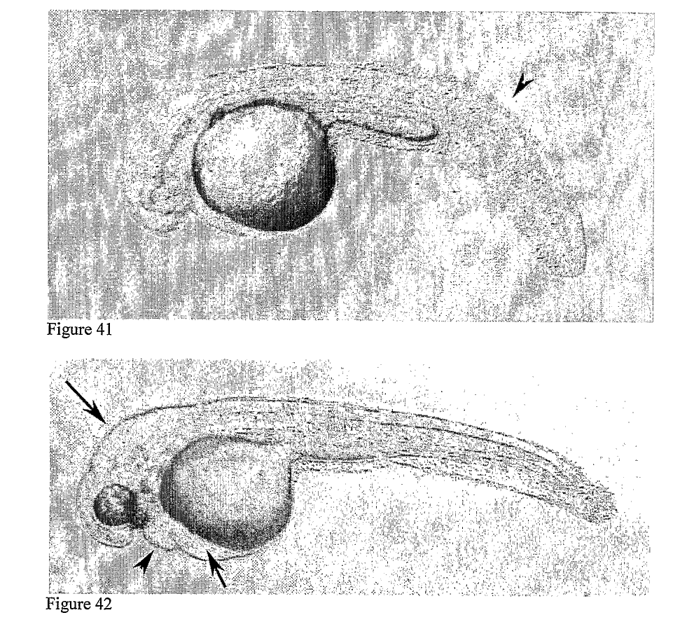

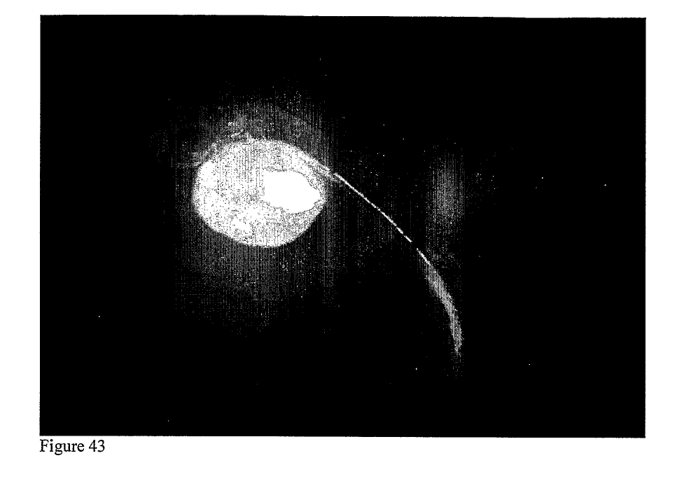

At 28 hpf embryos were observed morphologically. FIG. 41 shows a representative morphant embryo at 28 hpf. As indicated by the arrow, curly down body was found in 35% of the 20 morphants observed. At 56 hpf a total of 20 embryos were observed for phenotypic characteristics, a representative morphant embryo is shown in FIG. 42. As indicated by the long arrow, 60% of the embryos had cell death with an associated expanded hindbrain ventricle. Yolk sac edema was observed in 25% of embryos, as indicated by the short arrow. The arrowhead points out the lack of pericardial edema associated with the yolk sac edema.

Additional analyses were conducted on 48-56 hpf morphant embryos. A primary in situ hybridization screen with cdh5 on 19 morphants from the first dose group showed all were normal. The primary in situ hybridization screen with cdh5 on 10 morphants from the fourth dose group showed only 90% normal, the remaining 10% exhibiting low effects with curly tails. Microangiography on 19 morphants from the third dose group was used to locate the presence of FITC-Dextran in various regions of the embryo, see FIG. 43. Of the 19 embryos, none had FITC-Dextran in the heart and head but 16% had it in the heart alone. Reduced intersegmental vasculature was seen in 37% of the embryos. No leaky vasculature was observed. Normal embryos accounted for 47% of the sample.

Novel Applications Ascertained from in vivo Data

The present invention relates to the ten gene targets, and proteins related thereto, which were originally identified as upregulated during vasculogenesis or angiogenesis through microarray evaluation and subsequently proven to play a critical role in vivo with zebrafish embryo experimentation. These genes and proteins can form the basis of novel methods and treatments directed to angiogenesis-related conditions. For example, biological samples from a patient suspected of suffering from an angiogenesis-related condition can be screened to ascertain if genes or proteins of the present invention are expressed at the correct time, location, and intensity in the patient. Such screening methods form part of the claimed invention. If a gene and/or protein is identified as improperly expressed, therapies to correct the condition such as gene therapy or medicament can be initiated according to methods and procedures described herein or known in the art. With such specific data as is now possible using tools described herein, rapid diagnosis and specific, targeted treatment is possible.

One type of screening method envisioned relies on gene amplification for detecting patients with conditions related to vasculogenesis or angiogenesis. Such methods could employ PCR, in situ hybridisation, and/or Southern blotting techniques to elucidate the condition. Another type of screening method could be based on evaluations of gene expression, using known techniques such as quantitative PCR, microarrays, Northern blotting, or in situ hybridisation. Yet another type of screening method that could be used would measure or monitor protein expression and could be effected with techniques such as immunohistochemistry, Western blotting, ELISA, or FACS.

If it is determined, through methods of the present invention or other methods, that an angiogenesis-related condition could be improved through administration of compounds containing genes and/or proteins according to the invention, one or more of the genes and/or proteins could be administered together or sequentially by methods known in the art.

Isolated nucleic acid molecules or proteins of the present invention can be obtained, for example, by synthesis using standard direct peptide synthesizing techniques or recombinant methods. Proteins may be isolated or purified in a variety of ways known to those skilled in the art, such as electrophoretic purification or chromatographic techniques.

Administration of the compounds of the present invention can be effected by any method that enables delivery of the compounds to the site of desired action. These methods include oral routes, intraduodenal routes, parenteral injection (including intravenous, subcutaneous, intramuscular, intravascular or infusion), and topical administration.

Gene therapy approaches may be used to introduce nucleotides of the present invention into a cell, group of cells, or organism. Both in vivo and ex vivo methods can be utilized. Vectors typically are used in this procedure. Non-virus or virus vectors could be employed, for example recombinant adenovirus or retrovirus. According to this use, the desired gene is introduced into a DNA virus or RNA virus, such as avirulent retrovirus, adenovirus, adeno-associated virus, herpes virus, vaccinia virus, poxvirus, poliovirus, Sindbis virus, Sendai virus, SV40, and immunodeficiency virus (HIV). The recombinant virus is then infected into the target cell(s). Multiple genes could be incorporated in a single vector, alternatively, they could be introduced to the target cell(s) in separate vectors simultaneously or sequentially. These methods are known in the art and are described in numerous patents and publications.

Another means to interfere with gene expression or protein production contemplated by the present invention is to employ small interfering RNA (siRNA). siRNA comprises a sense and antisense strand of RNA corresponding to the gene of interest, for example, SEQ ID NO:2. A siRNA molecule consists of approximately 19 nucleotides plus an overhang of approximately 2 nucleotides at the 3′ end. Some preferred methods include between 19-23 nucleotides plus 3′ overhang. The siRNA is introduced to the cell or cells of interest through known methods. Following introduction, the cell or cells destroy ssRNA having the same sequence. This results in a reduction or prevention in translation of a targeted gene and a corresponding reduction or prevention in protein production.

The amount of active compound administered can be determined after assessing the subject being treated, the severity of the disorder or condition, the rate of administration, and the disposition of the compound. Doses may be administered all at once, or spread out over a discrete time period.

Compounds of the present invention may be applied as a sole therapy or may involve one or more other active medicinal or pharmaceutical agent. Compositions may include carriers, adjuvants, buffers, or excipients as known in the art. If desired, the compositions may further contain ingredients such as flavorings, sweeteners, binders, dyes, lubricating agents, perfume, thickening agents, stabilizers, emulsifiers, dispersants, suspending agents, preservatives, and pH regulating agents. Compositions may be in any suitable form, for example, tablet, capsule, pill, powder, sustained release formulation, solution, suspension, emulsion, ointment or cream. The compositions may be sterile. Methods of preparing various pharmaceutical compositions with a specific amount of active compound are known or apparent to those skilled in this art. The pharmaceutical compositions of the present invention that have been described can be applied to all diseases that require vasculogenic or angiogenic therapy.

For example, one method for the treatment of an angiogenesis-related disorder involves a composition according to the present invention used to vascularize ischemic tissue. There are many ways to determine if a tissue is at risk of suffering ischemic damage from undesirable vascular occlusion. Such methods are well known in the art and include, for example, imaging techniques such as MRI to evaluate myocardial disease. After determining where and when to apply compositions of the present invention, the compositions can be administered to increase angiogenesis in tissue affected by or at risk of being affected by a vascular occlusion. This could be an effective means of preventing and/or attenuating ischemia in such tissue. Methods are known in the art to evaluate and measure the degree to which ischemia has been attenuated.

Further treatment methods according to the present invention include the use of any known technique that permits visualization, measurement, and/or evaluation of the functionality and degree of ischemia of the patient's heart. Such evaluations could be made prior to initiating treatment, during the course of treatment, after treatment has been completed, or at some or all stages. Examples of such techniques include echocardiography, cardiovascular nuclear imaging, magnetic resonance imaging, and contrast angiography.

Although the present invention takes a step forward in the understanding of vasculogenesis and angiogenesis, and treatments for conditions related to the same, there is still a need in the art to further understand these conditions. Therefore, the present invention further contemplates the creation and use of non-human transgenic animals which could be used for analysis and experimentation. Transgenic animals containing mutant, knock-out or modified genes corresponding to those disclosed herein are therefore also included in the invention. Transgenic animals are genetically modified animals into which recombinant, exogenous or cloned genetic material has been experimentally transferred. Such genetic material is often referred to as a transgene. The nucleic acid sequence of the transgene may be integrated either at a locus of a genome where that particular nucleic acid sequence is not otherwise normally found or at the normal locus for the transgene. The transgene may consist of nucleic acid sequences derived from the genome of the same species or of a different species than the species of the target animal.

Transgenic animals can be produced by a variety of different methods including transfection, electroporation, microinjection, gene targeting in embryonic stem cells and recombinant viral and retroviral infection as known in the art. The method of introduction of nucleic acid fragments into recombination competent mammalian cells can be by any method that favors co-transformation of multiple nucleic acid molecules. Detailed procedures for producing transgenic animals are available to one skilled in the art, for example, U.S. Pat. Nos. 5,489,743 and 5,602,307.

Transgenic technology can be used to produce animals which lack one or more of the ten genes described above. Such knockout animals can be used, especially when their growth and development is measured against data from a wild type or control animal, to elucidate timing and function of the deleted gene(s). Further, these animals could also be engineered to exhibit angiogenesis-related disease states, thus furthering the understanding of the role of the particular gene(s) in the progression of the selected disease. This knowledge would be an advance in the state of the art and could lead to promising new therapies for the prevention, management, and cure of disease.

Further uses of transgenic animals according to the present invention include replacement of one or more of the above-identified gene(s) in the research organism with the human homolog of the gene. For example, a transgenic mouse whose gene corresponding to SEQ ID NO:7 has been replaced with the human homolog, SEQ ID NO:9. While it is accepted that research into effective drug therapies can be conducted in animal models, such a transgenic mouse could be a more effective screening tool into potential drug candidates for human use.

1. An isolated nucleic acid molecule according to any one of SEQ ID NO:s 2, 4, 7, 9, 12, 14, 17, 19, 21, 24, 26, 29, 31, 34, 36, 39, 41, 44, 46, 49, or 51, a fragment or analogue thereof, or an isolated nucleic acid molecule which hybridizes to one of the foregoing sequences under stringent conditions and which has the ability to stimulate or inhibit one or more of vasculogenesis, angiogenesis, vascular permeability, endothelial cell proliferation, endothelial cell differentiation, endothelial cell migration, or endothelial cell survival.

2. An isolated nucleic acid molecule which hybridizes to a compliment of a nucleic acid molecule according to claim 1 under stringent conditions.

3. An isolated siRNA molecule targeted to an isolated nucleic acid molecule according to claim 1, wherein the isolated siRNA molecule is at least 19 base pairs long.

4. An expression vector comprising the isolated nucleic acid according to claim 2, wherein the nucleic acid may be operatively associated with a regulatory nucleic acid controlling the expression of the polypeptide encoded by the nucleic acid.

5. A host cell genetically engineered to contain the isolated nucleic acid according to claim 1.

6. A host cell transfected with an expression vector according to claim 4.

7. A method of treating an angiogenesis-related condition in a cell, group of cells, or organism, comprising the step of administering an expression vector according to claim 4 to the cell, group of cells, or organism.

8. An antibody with specific reactivity to a nucleic acid according to claim 1, wherein the antibody may preferably be polyclonal or monoclonal and wherein the antibody may further comprise a detectable label such as a fluorescent label.

9. A transgenic, non-human animal which has been genetically engineered to contain a transgene comprising a nucleic acid according to claim 1, so that the transgene may be expressed.

10. A pharmaceutical composition comprising an isolated nucleic acid sequence according to claim 1.

11. A method of affecting vasculogenesis or angiogenesis in a cell, group of cells, or organism, comprising administering a pharmaceutical composition according to claim 16 to the cell, group of cells, or organism, wherein the pharmaceutical composition causes an increase or decrease in the cell, group of cells, or organism, and wherein the organism has an angiogenesis-related disorder such as cancer, retinopathy, macular degeneration, corneal ulceration, stroke, ischemic heart disease, infertility, ulcers, scleroderma, wound healing, ischemia, ischemic heart disease, myocardial infarction, myocardosis, angina pectoris, unstable angina, coronary arteriosclerosis, arteriosclerosis obliterans, Berger's disease, arterial embolism, arterial thrombosis, cerebrovascular occlusion, cerebral infarction, cerebral thrombosis, cerebral embolism, rubeosis proliferative vitreoretinopathy, chronic inflammation, inflammatory bowel disease, psoriasis, sarcoidosis or rheumatoid arthritis.

12. An isolated polypeptide comprising a sequence of amino acids substantially corresponding to an amino acid sequence in any one of SEQ ID NO:s 3, 5, 8, 10, 13, 15, 18, 20, 22, 25, 27, 30, 32, 35, 37, 40, 42, 45, 47, 50, and 52, or a fragment or analogue thereof, the isolated polypeptide having the ability to affect angiogenesis in a cell, a group of cells, or an organism.

13. A host cell genetically engineered to express an isolated polypeptide according to claim 12.

14. An antibody specifically reactive with a polypeptide according to claim 12, wherein the antibody may be polyclonal or monoclonal, and wherein the antibody may further comprise a detectable label such as a fluorescent label.

15. A transgenic, non-human animal which has been genetically engineered to contain a transgene comprising a nucleic acid which encodes an isolated polypeptide according to claim 12 so that the transgene may be expressed.

16. A pharmaceutical composition comprising an isolated polypeptide according to claim 12.

17. A method of causing vasculogenesis or angiogenesis in a cell, group of cells, or organism, comprising the step of administering a pharmaceutical composition according to claim 16 to the cell, group of cells, or organism, the affecting may preferably cause an increase or decrease, more preferably, the cell, group of cells, or organism that has an angiogenesis-related disorder such as cancer, retinopathy, macular degeneration, corneal ulceration, stroke, ischemic heart disease, infertility, ulcers, scleroderma, wound healing, ischemia, ischemic heart disease, myocardial infarction, myocardosis, angina pectoris, unstable angina, coronary arteriosclerosis, arteriosclerosis obliterans, Berger's disease, arterial embolism, arterial thrombosis, cerebrovascular occlusion, cerebral infarction, cerebral thrombosis, cerebral embolism, rubeosis proliferative vitreoretinopathy, chronic inflammation, inflammatory bowel disease, psoriasis, sarcoidosis, or rheumatoid arthritis.

18. A method of detecting an angiogenesis-related transcript in a cell of a patient, the method comprising contacting a biological sample from the patient with a polynucleotide that selectively hybridizes to a sequence at least 80% identical to a sequence according to any one of SEQ ID NO:s 2, 4, 7, 9, 12, 14, 17, 19, 21, 24, 26, 29, 31, 34, 36, 39, 41, 44, 46, 49, and 51, wherein an angiogenesis-related transcript is detected where hybridization is detected, wherein the polynucleotide comprises a sequence according to any one of SEQ ID NO:s 2, 4, 7, 9, 12, 14, 17, 19, 21, 24, 26, 29, 31, 34, 36, 39, 41, 44, 46, 49, and 51, wherein the biological sample is a tissue sample or is comprised of isolated nucleic acids such as mRNA, wherein the nucleic acids are amplified prior to the step of contacting the biological sample with the polynucleotide, preferably and wherein the polynucleotide is immobilized on a solid surface.

19. A method of affecting angiogenesis and/or vasculogenesis in a vertebrate organism, the method comprising administering to the organism an effective angiogenesis and/or vasculogenesis affecting amount of a nucleotide according to claim 1, wherein the organism is preferably a mammal such as mice, rats, rabbits, guinea pigs, cats, dogs, pigs, cows, monkeys, and humans, wherein vasculogenesis or angiogenesis is enhanced, increased, inhibited, or decreased, and wherein the organism preferably has an angiogenesis-related disorder such as cancer, retinopathy, macular degeneration, corneal ulceration, stroke, ischemic heart disease, infertility, ulcers, scleroderma, wound healing, ischemia, ischemic heart disease, myocardial infarction, myocardosis, angina pectoris, unstable angina, coronary arteriosclerosis, arteriosclerosis obliterans, Berger's disease, arterial embolism, arterial thrombosis, cerebrovascular occlusion, cerebral infarction, cerebral thrombosis, cerebral embolism, rubeosis proliferative vitreoretinopathy, chronic inflammation, inflammatory bowel disease, psoriasis, sarcoidosis or rheumatoid arthritis.

20. A transgenic increased or decreased angiogenesis laboratory animal comprising one or more cells in which the expression of a sequence according to any one of SEQ ID NO:s 2, 4, 7, 9, 12, 14, 17, 19, 21, 24, 26, 29, 31, 34, 36, 39, 41, 44, 46, 49, and 51 is upregulated, downregulated, or absent.

21. A method of affecting angiogenesis and/or vasculogenesis in a vertebrate organism, the method comprising administering to the organism an effective angiogenesis and/or vasculogenesis affecting amount of a polypeptide according to claim 12, wherein the organism is preferably a mammal such as mice, rats, rabbits, guinea pigs, cats, dogs, pigs, cows, monkeys, and humans, wherein vasculogenesis or angiogenesis is enhanced, increased, inhibited, or decreased, and wherein the organism preferably has an angiogenesis-related disorder such as cancer, retinopathy, macular degeneration, corneal ulceration, stroke, ischemic heart disease, infertility, ulcers, scleroderma, wound healing, ischemia, ischemic heart disease, myocardial infarction, myocardosis, angina pectoris, unstable angina, coronary arteriosclerosis, arteriosclerosis obliterans, Berger's disease, arterial embolism, arterial thrombosis, cerebrovascular occlusion, cerebral infarction, cerebral thrombosis, cerebral embolism, rubeosis proliferative vitreoretinopathy, chronic inflammation, inflammatory bowel disease, psoriasis, sarcoidosis, or rheumatoid arthritis.
